(19) United States
(12) Patent Application Publication
(10) Pub. No.: US 2026/0125372 A1
(43) Pub. Date: May 7, 2026

(54) DYNAMIN-1-LIKE PROTEIN INHIBITORS AND USES THEREOF

(71) Applicant: Astellas Engineered Small Molecules US, Incorporated, Cambridge, MA (US)

(72) Inventors: Bin Ma, Cambridge, MA (US); Takeru Furuya, Cambridge, MA (US)

(21) Appl. No.: 19/117,763

(22) PCT Filed: Oct. 24, 2023
(86) PCT No.: PCT/US2023/035789

§ 371 Date: Apr. 2, 2025

Related U.S. Application Data
(60) Provisional application No. 63/419,132, filed on Oct. 25, 2022.

(51) Int. Cl. C07D 417/14; A61K 31/4709; C07D 405/14; C07D 417/04

(52) U.S. Cl. CPC C07D 417/14; A61K 31/4709; C07D 405/14; C07D 417/04

(57) ABSTRACT

Provided are compounds of the Formula (I); or pharmaceutically acceptable salts thereof, which can be useful for the inhibition of Drp 1 and in the treatment of a variety of Drp 11 mediated conditions or diseases.

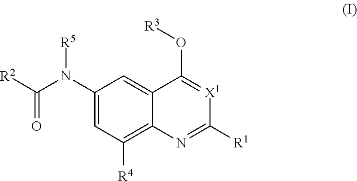

### RELATED APPLICATIONS

This application claims priority to U.S. Provisional Application No. 63/419,132, filed on Oct. 25, 2022. The entire contents of the foregoing application are expressly incorporated herein by reference.

### FIELD OF THE INVENTION

The present disclosure relates to inhibitors of dynamin-1-like protein (Drp1), and pharmaceutically acceptable salts thereof, compositions of these compounds, processes for their preparation, and their use in the treatment of diseases.

### BACKGROUND OF THE INVENTION

Dynamin-1-like protein is a GTPase that regulates mitochondrial fission. In humans, dynamin-1-like protein, which is typically referred to as dynamin-related protein 1 (Drp1), is encoded by the DNM1L gene. Inhibitors of Drp1 blocks cell death, implicating mitochondrial fission as an important step in apoptosis. Drp1 sequences (OMIM 603850) are publically available, for example, from GenBank® sequence database (e.g., BAA22193 (human, protein, AB006965 (human, nucleic acid)).

In healthy cells, fusion and fission events participate in regulating mitochondrial morphology. Drp1, a dynamin-related protein, mediates outer mitochondrial membrane fission and typically activated during cell division. Specifically during the M phase, Drp1 is activated which induces mitochondrial fission to ensure equal distribution of mitochondria to each daughter cell. Mitochondrial fission creates smaller mitochondria which are more capable of generating reaction oxygen species, facilitating mitophagy, or accelerating cell proliferation compared to their larger counterparts (Archer S L. Mitochondrial dynamics—mitochondrial fission and fusion in human diseases. N Engl J Med. 2013 369(23):2236-51). Upon induction of apoptosis, Drp1 translocates from the cytosol to mitochondria, where it preferentially localizes to potential sites of organelle division. Drp1 is regulated by phosphorylation, with phosphorylation at serine 616 increasing activity and phosphorylation at serine 637 decreasing activity. Inhibition of Drp1 prevents the loss of the mitochondrial membrane potential and the release of cytochrome c, and reveals a reproducible swelling of the organelles. Remarkably, inhibition of Drp1 blocks cell death, implicating mitochondrial fission as an important step in cellular apoptosis. Further, inhibition of Drp1 has been shown to decrease reactive oxygen species due to a reduction in mitochondrial fission. Thus, modulation Drp1 activity is effective in the treatment of a variety of conditions, such as, for example, cardiovascular disease, kidney disease, ophthalmic conditions, cancer, ischemia-reperfusion injury, and neurodegenerative and cognitive diseases. Indeed, Drp1 is an important biological target for compounds used to help treat and prevent diseases such as cardiovascular disease, kidney disease, ophthalmic conditions, cancer, cognitive disease, and other related conditions.

Thus, there is a need for Drp1 inhibitors as potential therapeutic agents for treating diseases or disorders that are responsive to Drp1 inhibition.

### SUMMARY OF THE INVENTION

The present disclosure provides compounds that are Drp1 inhibitors. In a first aspect, the present disclosure relates to compounds having the Formula I:

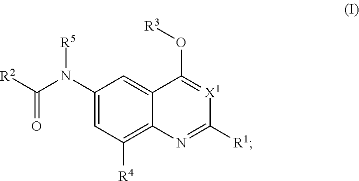

or a pharmaceutically acceptable salt thereof, wherein:
X¹ is CH or N;
R¹ is a 5- to 10-membered monocyclic or bicyclic heteroaryl optionally substituted with one more R^{1a};
each R^{1a} is independently selected from the group consisting of halo, cyano, C₁₋₆alkyl, C₁₋₆haloalkyl, C₃₋₆cycloalkyl, OH, C₁₋₆alkoxy, and C₁₋₆haloalkoxy;
R² is C₁₋₆alkyl, C₃₋₆cycloalkyl, bridged C₅₋₁₂cycloalkyl, phenyl, 3- to 10-membered monocyclic or bicyclic heterocyclyl, or 5- to 10-membered monocyclic or bicyclic heteroaryl, each of which is optionally substituted by one or more R^{2a};
each R^{2a} is independently selected from halo, cyano, C₁₋₆alkyl, C₁₋₆haloalkyl, OH, C₁₋₆alkoxy, C₁₋₆haloalkoxy, —C(O)R^{2b}, —C(O)OR^{2b}, —C(O)NR^{N2a}R^{N2b}, and —NR^{N2a}R^{N2b};
R^{2b}, R^{N2a}, and R^{N2b} are each independently H, C₁₋₆alkyl, or C₁₋₆haloalkyl;
R³ is C₁₋₆alkyl, C₃₋₆cycloalkyl, phenyl, 3- to 10-membered monocyclic or bicyclic heterocyclyl, or 5- to 10-membered monocyclic or bicyclic heteroaryl, each of which is optionally substituted with one or more R^{3a};
each R^{3a} is independently selected from the group consisting of halo, C₁₋₆alkyl, OH, C₁₋₆alkoxy, and C₁₋₆haloalkoxy;
R⁴ is H, C₁₋₆alkyl, C₁₋₆haloalkyl, or C₃₋₆cycloalkyl; and
R⁵ is H, C₁₋₆alkyl, or C₁₋₆haloalkyl.

Another aspect of the disclosure relates to pharmaceutical compositions comprising compounds of Formula (I) or pharmaceutically acceptable salts thereof, and a pharmaceutical carrier.

In yet another aspect, the present disclosure provides a method of treating a Drp1 mediated disease or disorder in a subject comprising administering to said subject an effective amount of a compound described herein or a pharmaceutically acceptable salt thereof or pharmaceutical compositions comprising a compound described herein or pharmaceutically acceptable salts thereof, and a pharmaceutical carrier. In some embodiments, the method is for the treatment of cancer.

Another aspect of the present disclosure relates to the use of an effective amount of a compound described herein or a pharmaceutically acceptable salt thereof, for the manufacture of a medicament for the treatment of a Drp1 mediated disease or disorder. Also provided is a compound described herein or a pharmaceutically acceptable salt thereof for use in treating a Drp1 mediated disease or disorder.

Another aspect of the disclosure relates to pharmaceutical compositions comprising a compound described herein or pharmaceutically acceptable salts thereof, and a pharmaceutical carrier for treating a Drp1 mediated disease or disorder, especially cancer.

### DETAILED DESCRIPTION OF THE INVENTION

Herein, the phrase “Drp1 inhibitor” refers to substances that effectively reduce the activity of Drp1. Substances can be tested for their Drp1 activity by contacting the substance with cells expressing Drp1, detecting their binding with Drp1 and then detecting signals that serve as the indicator of the deactivation of Drp1 (see Biological Example 1).

The present disclosure provides compounds and pharmaceutical compositions thereof that may be useful in the treatment of diseases or disorders through mediation of Drp1 function/activity. In some embodiments, the compounds of present disclosure are Drp1 inhibitors.

### Compounds and Compositions

In a first embodiment, the present disclosure provides a compound of Formula (I):

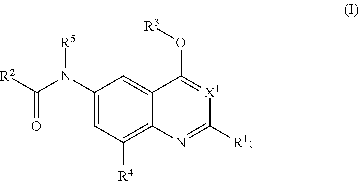

or a pharmaceutically acceptable salt thereof, therein the variables in Formula (I) are as defined in the first aspect above.

In a second embodiment, the compound of the present disclosure is represented by Formula (IA):

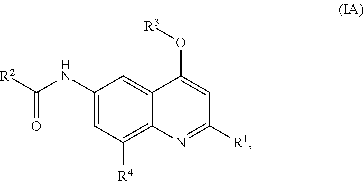

or a pharmaceutically acceptable salt thereof; wherein the variables R¹, R², R³, and R⁴ depicted in Formula (IA) are as described in the first embodiment.

In a third embodiment, for the compounds of Formula (I) or (IA), or a pharmaceutically acceptable salt thereof, R⁴ is H or C₁₋₃alkyl; and the remaining variables are as described in the first or second embodiment.

In a fourth embodiment, for the compounds of Formula (I) or (IA), or a pharmaceutically acceptable salt thereof, R⁴ is H or —CH₃; and the remaining variables are as described in the third embodiment. In a specific embodiment, for the compounds of Formula (I) or (IA), or a pharmaceutically acceptable salt thereof, R⁴ is H; and the remaining variables are as described in the third embodiment.

In a fifth embodiment, for the compounds of Formula (I) or (IA), or a pharmaceutically acceptable salt thereof, R¹ is a 5- or 6-membered monocyclic heteroaryl, wherein the 5- or 6-membered monocyclic heteroaryl is optionally substituted by 1 or 2 R^{1a}; each R^{1a} is independently selected from halo, cyano, C₁₋₄alkyl, C₃₋₄cycloalkyl, and C₁₋₃alkoxy; and the remaining variables are as described in the first, second, third, or fourth embodiment. In a specific embodiment, R¹ is a 5-membered monocyclic heteroaryl optionally substituted by 1 or 2 R^{1a}; each R^{1a} is independently selected from the group consisting of halo, cyano, C₁₋₄alkyl, C₃₋₄cycloalkyl, and C₁₋₃alkoxy; and the remaining variables are as described in the first, second, third, or fourth embodiment.

In a sixth embodiment, for the compounds of Formula (I) or (IA), or a pharmaceutically acceptable salt thereof, R¹ is selected from the group consisting of imidazoyl, pyrazoyl, triazoyl, and thiazoyl, each of which is optionally substituted by 1 or 2 R^{1a}; and the remaining variables are as described in the fifth embodiment.

In a seventh embodiment, for the compounds of Formula (I) or (IA), or a pharmaceutically acceptable salt thereof, R¹ is represented by the following structural formula:

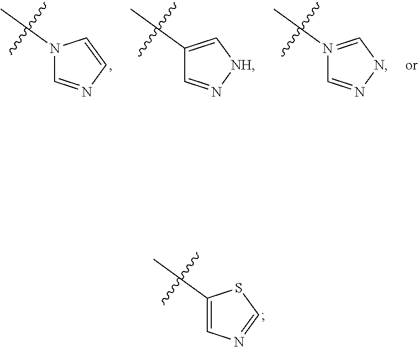

each of which is optionally substituted by 1 or 2 R^{1a}, each R^{1a} is independently selected from the group consisting of —CH₃, —CH₂CH₃, —CH₂CH₂CH₃, —C(CH₃)₃, cyclopropyl, —OCH₃, —Cl, and cyano; and the remaining variables are as described in the sixth embodiment.

In an eighth embodiment, for the compounds of Formula (I) or (IA), or a pharmaceutically acceptable salt thereof, R¹ is selected from the group consisting of imidazoyl and thiazoyl, each of which is optionally substituted by 1 or 2 R^{1a}, each R^{1a} is independently selected from the group consisting of —CH₃, cyclopropyl, and —OCH₃; and the remaining variables are as described in the fifth embodiment.

In a ninth embodiment, for the compounds of Formula (I) or (IA), or a pharmaceutically acceptable salt thereof, R² is C₁₋₃alkyl, C₃₋₆cycloalkyl, bridged C₅₋₈cycloalkyl, 3- to 6-membered monocyclic heterocyclyl, or 5- or 6-membered monocyclic heteroaryl, each of which is optionally substituted by 1 to 3 R^{2a}, each R^{2a} is independently selected from the group consisting of halo, cyano, C₁₋₃alkyl, C₁₋₃haloalkyl, —OH and C₁₋₃ alkoxy; and the remaining variables are as described in the first, second, third, fourth, fifth, sixth, seventh, or eighth embodiment.

In a tenth embodiment, for the compounds of Formula (I) or (IA), or a pharmaceutically acceptable salt thereof, R² is C₁₋₃alkyl, cyclopropyl, cyclobutyl, bicyclo[1.1.1]pentanyl, oxetanyl, tetrahydropyranyl, or isoxazoyl, each of which is optionally substituted by 1 to 3 R^{2a}; and the remaining variables are as described in the ninth embodiment.

In an eleventh embodiment, for the compounds of Formula (I) or (IA), or a pharmaceutically acceptable salt thereof, R² is C₁₋₃alkyl optionally substituted by 1 to 3 R^{2a}, or R² is represented by the following structural formula:

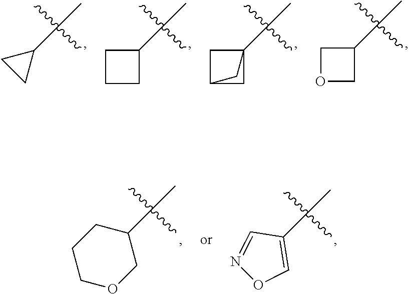

each of which is optionally substituted by 1 or 2 R^{2a}, each R^{2a} is selected from the group consisting of —F, cyano, —CH₃, —CF₃, —OH, —OCH₃, —OCH₂CH₃, and —OCH₂CH₂CH₃; and the remaining variables are as described in the tenth embodiment.

In a twelfth embodiment, for the compounds of Formula (I) or (IA), or a pharmaceutically acceptable salt thereof, R² is —CF₃, —CH₂CH₂OCH₃, —CH₂OCH₃, —CH₂OH, —CH(OH)CH₃, —CH(CH₃)CH₂OCH₃, —CH(CH₃)CN, —CH(CH₃)OCH₃, —CH(CH₃)OCH₂CH₃, —CH(CH₃)OCH₂CH₂CH₃, —CF₂CH₂OH, or R² is represented by the following structural formula:

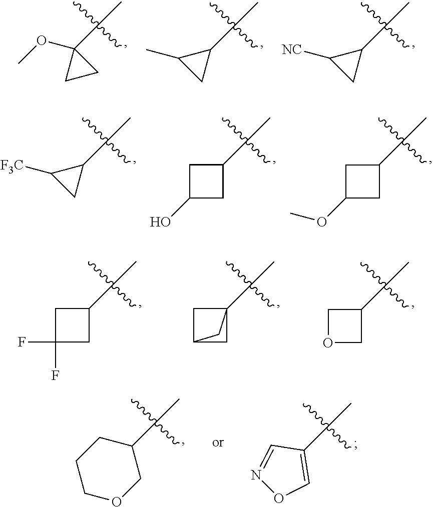

and the remaining variables are as described in the eleventh embodiment.

In a thirteenth embodiment, for the compounds of Formula (I) or (IA), or a pharmaceutically acceptable salt thereof, R² is C₁₋₃alkyl, cyclopropyl, cyclobutyl, oxetanyl, or tetrahydropyranyl, wherein the C₁₋₃alkyl, cyclopropyl, and cyclobutyl are each optionally substituted with 1 to 3 R^{2a}; and the remaining variables are as described in the ninth embodiment.

In a fourteenth embodiment, for the compounds of Formula (I) or (IA), or a pharmaceutically acceptable salt thereof, R² is —CH(CH₃)CN, —CH(CH₃)OCH₃, —CH(CH₃)OCH₂CH₃, —CH(CH₃)OCH₂CH₂CH₃, or R² is represented by the following structural formula:

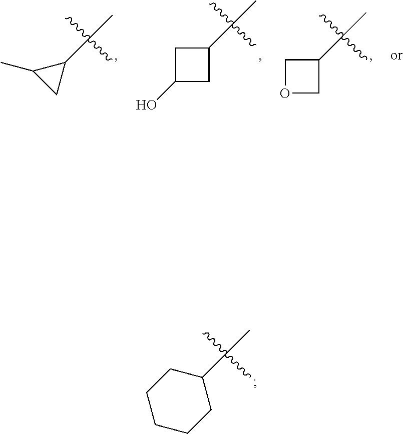

and the remaining variables are as described in the thirteenth embodiment.

In a fifteenth embodiment, for the compounds of Formula (I) or (IA), or a pharmaceutically acceptable salt thereof, R³ is C₁₋₄alkyl or C₃₋₄cycloalkyl, each of which is optionally substituted with 1 to 3 R^{3a}; each R^{3a} is independently selected from the group consisting of C₁₋₃alkyl, —OH, and C₁₋₃alkoxy; and the remaining variables are as described in the first, second, third, fourth, fifth, sixth, seventh, eighth, ninth, tenth, eleventh, twelfth, thirteenth, or fourteenth embodiment.

In a sixteenth embodiment, for the compounds of Formula (I) or (IA), or a pharmaceutically acceptable salt thereof, R³ is C₁₋₄alkyl or cyclopropyl, wherein the C₁₋₃alkyl is optionally substituted with 1 to 3 R^{3a}; each R^{3a} is independently selected from —CH₃, OH, and —OCH₃; and the remaining variables are as described in the fifteenth embodiment.

In a seventeenth embodiment, for the compounds of Formula (I) or (IA), or a pharmaceutically acceptable salt thereof, R³ is —CH₃, —CH₂CH₃, —CH₂CH₂OH, —CH₂CH₂OCH₃, —CH(CH₃)CH₂OCH₃, —C(CH₃)₂CH₂OCH₃, or cyclopropyl; and the remaining variables are as described in the sixteenth embodiment. In a specific embodiment, for the compounds of Formula (I) or (IA), or a pharmaceutically acceptable salt thereof, R³ is —CH₂CH₃, —CH₂CH₂OH, —CH₂CH₂OCH₃, or —CH(CH₃)CH₂OCH₃; and the remaining variables are as described in the sixteenth embodiment.

In an eighteenth embodiment, the present disclosure provides a compound described herein (e.g., a compound of any one of Examples 1-54), or a pharmaceutically acceptable salt thereof.

In a nineteenth embodiment, the present disclosure provides a compound selected from the group consisting of:

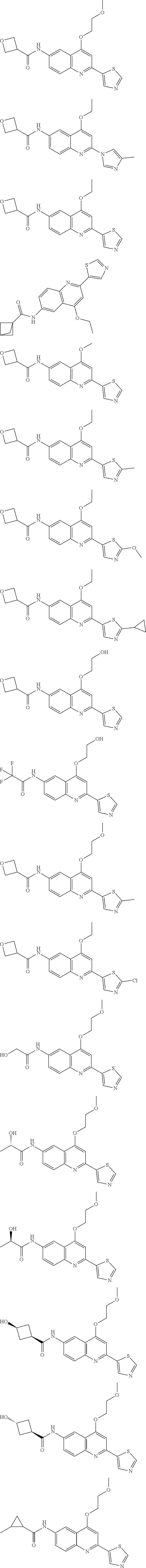

[chemical structure]

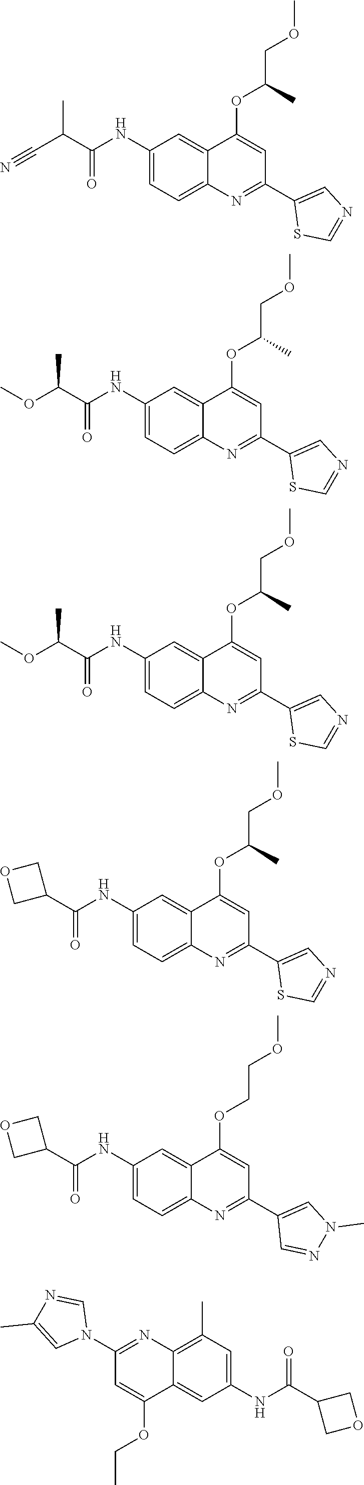

or a pharmaceutically acceptable salt thereof.

The compounds and intermediates described herein may be isolated and used as the neutral form. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Deuterated compounds of Formula (I) or (IA) can generally be prepared by conventional techniques known to those skilled in the art or by processes analogous to those described in the accompanying Examples and Preparations using appropriate deuterated reagents in place of the non-labeled reagent previously employed. In one embodiment, the present disclosure provides deuterated compounds described herein or a pharmaceutically acceptable salt thereof.

Another embodiment of the disclosure is a compound disclosed herein, including a compound of Formula (I) or (IA), or a compound in the Exemplification, or a pharmaceutically acceptable salt of any of the foregoing, in which one or more hydrogen atoms is replaced with deuterium. The deuterium enrichment at any one of the sites where hydrogen has been replaced by deuterium is at least 50%, 75%, 85%, 90%, 95%, 98% or 99%. Deuterium enrichment is a mole percent and is obtained by dividing the number of compounds with deuterium enrichment at the site of enrichment with the number of compounds having hydrogen or deuterium at the site of enrichment.

Pharmaceutically acceptable solvates in accordance with the disclosure include those wherein the solvent of crystallization may be isotopically substituted, e.g. D₂O, d₆-acetone, d₆-DMSO.

It will be recognized by those skilled in the art that the compounds of the present disclosure may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

The present disclosure also provides a pharmaceutical composition comprising a compound described herein (e.g., a compound according to any one of the preceding embodiments), or a pharmaceutically acceptable salt thereof, and one or more pharmaceutically acceptable carriers.

### Methods of Use

The compounds described herein have Drp1 inhibitory activity. As used herein, “Drp1 inhibitory activity” refers to the ability of a compound or composition to induce a detectable decrease in Drp1 activity in vivo or in vitro.

In certain embodiments, the present disclosure provides a method of treating a disease or disorder responsive to inhibition of Drp1 activity (referred herein as “Drp1 mediated disease or disorder”) in a subject in need of the treatment. The method comprises administering to the subject a compound described herein (e.g., a compound described in any one of the first to nineteenth embodiments) or a pharmaceutically acceptable salt thereof or a pharmaceutical composition thereof.

In certain embodiments, the present disclosure provides the use of a compound described herein (e.g., a compound described in any one of the first to nineteenth embodiments) or a pharmaceutically acceptable salt thereof or a pharmaceutical composition comprising a compound described herein or a pharmaceutically acceptable salt thereof for the manufacture of a medicament for the treatment of a Drp1 mediated disease or disorder in a subject in need of the treatment.

In certain embodiments, the present disclosure provides a compound described herein (e.g., a compound described in any one of the first to nineteenth embodiments) or a pharmaceutically acceptable salt thereof or a pharmaceutical composition comprising a compound described herein or a pharmaceutically acceptable salt thereof for use in the treatment of a Drp1 mediated disorder or disease in a subject in need of the treatment.

In certain embodiments, the present disclosure provides a pharmaceutical composition comprising a compound described herein (e.g., a compound described in any one of the first to nineteenth embodiments) or a pharmaceutically acceptable salt thereof for treating Drp1 mediated disorder.

In certain embodiments, the present disclosure provides the use of a compound described herein (e.g., a compound described in any one of the first to nineteenth embodiments) or a pharmaceutically acceptable salt thereof for the treatment of a Drp1 mediated disease or disorder in a subject in need of the treatment.

In certain embodiments, the Drp1 mediated disease or disorder is a muscle structure disorder, a neuronal activation disorder, a muscle fatigue disorder, a muscle mass disorder, a beta oxidation disease, a metabolic disease, a cancer, a vascular disease, an ocular vascular disease, a muscular eye disease, or a renal disease.

In certain embodiments:
the muscle structure disorder is selected from the group consisting of Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorders, myotonic chondrodystrophy, myotonic dystrophy, myotubular myopathy, nemaline body disease, oculopharyngeal MD, and stress urinary incontinence;
the neuronal activation disorder is selected from the group consisting of amyotrophic lateral sclerosis, Charcot-Marie-Tooth disease, Guillain-Barre syndrome, Lambert-Eaton syndrome, multiple sclerosis, myasthenia gravis, nerve lesion, peripheral neuropathy, spinal muscular atrophy, tardy ulnar nerve palsy, and toxic myoneural disorder;
the muscle fatigue disorder is selected from the group consisting of chronic fatigue syndrome, diabetes (type I or II), glycogen storage disease, fibromyalgia, Friedreich's ataxia, intermittent claudication, lipid storage myopathy, MELAS, mucopolysaccharidosis, Pompe disease, and thyrotoxic myopathy;
the muscle mass disorder is selected from the group consisting of cachexia, cartilage degeneration, cerebral palsy, compartment syndrome, critical illness myopathy, inclusion body myositis, muscular atrophy (disuse), sarcopenia, steroid myopathy, and systemic lupus erythematosus;
the beta oxidation disease is selected from the group consisting of systemic carnitine transporter, carnitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency, and riboflavin-responsive disorders of β-oxidation (RR-MADD);
the metabolic disease is selected from the group consisting of hyperlipidemia, dyslipidemia, hyperchlolesterolemia, hypertriglyceridemia, HDL hypocholesterolemia, LDL hypercholesterolemia and/or HLD non-cholesterolemia, VLDL hyperproteinemia, dyslipoproteinemia, apolipoprotein A-I hypoproteinemia, atherosclerosis, disease of arterial sclerosis, disease of cardiovascular systems, cerebrovascular disease, peripheral circulatory disease, metabolic syndrome, syndrome X, obesity, diabetes (type I or II), hyperglycemia, insulin resistance, impaired glucose tolerance, hyperinsulinism, diabetic complication, cardiac insufficiency, cardiac infarction, cardiomyopathy, hypertension, Non-alcoholic fatty liver disease (NAFLD), Nonalcoholic steatohepatitis (NASH), thrombus, Alzheimer's disease, neurodegenerative diseases including Parkison's disease, demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease, and pancreatitis;
the vascular disease is selected from the group consisting of peripheral vascular insufficiency, peripheral vascular disease, intermittent claudication, peripheral vascular disease (PVD), peripheral artery disease (PAD), peripheral artery occlusive disease (PAOD), and peripheral obliterative arteriopathy;
the ocular vascular disease is selected from the group consisting of age-related macular degeneration (AMD), stargardt disease, hypertensive retinopathy, diabetic retinopathy, retinopathy, macular degeneration, retinal haemorrhage, and glaucoma;
the muscular eye disease is selected from the group consisting of strabismus, progressive external ophthalmoplegia, esotropia, exotropia, a disorder of refraction and accommodation, hypermetropia, myopia, astigmatism, anisometropia, presbyopia, a disorders of accommodation, and internal ophthalmoplegia; and
the renal disease is selected from the group consisting of glomerulonephritis, glomerulosclerosis, nephrotic syndrome, hypertensive nephrosclerosis, acute nephritis, recurrent hematuria, persistent hematuria, chronic nephritis, rapidly progressive nephritis, acute renal failure, chronic renal failure, diabetic nephropathy, and Bartter's syndrome.

In other embodiments, the Drp1 mediated disease or disorder is selected from the group consisting of genetic lipodystrophy, non-alcoholic fatty liver disease (NAFLD), non-alcoholic steatohepatitis (NASH), renal ischemia/reperfusion injury (IRI), Duchenne & Becker muscular dystrophy, diabetes (type I or type II), obesity, and sarcopenia.

In other embodiments, the Drp1 mediated disease or disorder is selected from the group consisting of inflammatory diseases of the bone, T Cell immune modulation, Neuropathic Pain, cancer, Alpers's Disease, CPEO-Chronic progressive external ophthalmoplegia, Kearns-Sayra Syndrome (KSS), Leber Hereditary Optic Neuropathy (LHON), MELAS-Mitochondrial myopathy, encephalomyopathy, lactic acidosis, and stroke-like episodes, MERRF-Myoclonic epilepsy and ragged-red fiber disease, NARP-neurogenic muscle weakness, ataxia, retinitis pigmentosa, Pearson Syndrome, platinum-based chemotherapy induced ototoxicity, Cockayne syndrome, xeroderma pigmentosum A, Wallerian degeneration, and HIV-induced lipodystrophy.

In other embodiments, the Drp1 mediated disease or disorder is selected from the group consisting of acute kidney injury, cardiac ischemia, pulmonary arterial hypertension, polycystic kidney disease, Huntington's disease, a neurodegenerative disease, or Charcot-Marie-Tooth disease.

In other embodiments, the Drp1 mediated disease or disorder is a neurodegenerative disease. In other embodiments, the neurodegenerative disease is Parkinson's disease.

The compounds, or pharmaceutically acceptable salts thereof described herein may be used to decrease the expression or activity of Drp1, or to otherwise affect the properties and/or behavior of Drp1 in a cell.

One embodiment of the present disclosure includes a method of decreasing the expression or activity of Drp1, or to otherwise affect the properties and/or behavior of Drp1 in a subject comprising administering to said subject an effective amount of at least one compound described herein, or a pharmaceutically acceptable salt thereof.

In certain embodiments, the present disclosure relates to the aforementioned methods, wherein said subject is a mammal.

In certain embodiments, the present disclosure relates to the aforementioned methods, wherein said subject is a primate.

In certain embodiments, the present disclosure relates to the aforementioned methods, wherein said subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 g and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered orally.

In certain embodiments, the present disclosure relates to the aforementioned methods, wherein said compound is administered parenterally.

In certain embodiments, the present disclosure relates to the aforementioned methods, wherein said compound is administered intramuscularly, intravenously, subcutaneously, pulmonary, rectally, intrathecally, topically or intranasally.

In certain embodiments, the present disclosure relates to the aforementioned methods, wherein said compound is administered systemically.

The compounds of the present disclosure are typically used as a pharmaceutical composition (e.g., a compound of the present disclosure and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present disclosure and a solvent (i.e., solvate) or water (i.e., hydrate).

The formulations may be prepared using conventional dissolution and mixing procedures. For example, the bulk drug substance (i.e., compound of the present disclosure or stabilized form of the compound (e.g., complex with a cyclodextrin derivative or other known complexation agent)) is dissolved in a suitable solvent in the presence of one or more of the excipients described above. The compound of the present disclosure is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In some embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to therapeutic treatment or prophylactic treatment. Treatment includes the management and care of a patient for the purpose of combating the disease, condition, or disorder and includes the administration of a compound of the present disclosure to obtaining desired pharmacological and/or physiological effect. Therapeutic treatment includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. Prophylactic treatment refers to reducing the likelihood of developing the symptoms or complications of the disease, condition or disorder.

As used herein, a subject is “in need of” a treatment if such subject would benefit biologically, medically or in quality of life from such treatment (in some embodiments, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. In some embodiments, the “one or more” substituents can be 1, 2, 3, 4, 5, 6, etc. substituents, each of which can the same or different. In some embodiment, the “one or more” substituents can be 1 to 6, 1 to 4, 1 to 3 or 1 to 2 substituents, each of which can the same or different.

As used herein, the term “alkyl” refers to a fully saturated branched or unbranched hydrocarbon moiety. The term “C₁₋₆alkyl” refers to an alkyl having 1 to 6 carbon atoms. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. In some embodiments, alkoxy groups have 1-6 carbons, 1-4 carbons, or 1-3 carbons, and in some embodiments about 1-2 carbons. The term “C₁₋₆alkoxy” refers to an alkoxy having 1 to 6 carbon atoms and the term “C₁₋₃alkoxy” refers to an alkoxy having 1 to 3 carbon atoms. The term “C₁₋₂ alkoxy” is to be construed accordingly.

The number of carbon atoms in a group is specified herein by the prefix “Cₓ₋ₓₓ”, wherein x and xx are integers. For example, “C₁₋₃alkyl” is an alkyl group which has from 1 to 3 carbon atoms.

“Halogen” or “halo” may be fluorine, chlorine, bromine or iodine.

As used herein, the term “haloalkyl” refers to an alkyl group as defined herein, wherein at least one of the hydrogen atoms is replaced by a halo atom. The term “C₁₋₆haloalkyl” refers to a haloalkyl group having 1 to 6 carbon atoms. The terms “C₁₋₄haloalkyl” and “C₁₋₃haloalkyl” are to be construed accordingly. The haloalkyl group can be monohaloalkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Typically the polyhaloalkyl group contains up to 13, or 12, or 11, or 10, or 9, or 8, or 7, or 6, or 5, or 4, or 3, or 2 halo groups. Non-limiting examples of C₁₋₆haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl group refers to an alkyl group having all hydrogen atoms replaced with halo atoms.

As used herein, the term “haloalkoxy” refers to an alkoxy group as defined herein, wherein at least one of the hydrogen atoms on the alkyl moiety is replaced by a halo atom. The term “C₁₋₆haloalkoxy” refers to a haloalkoxy group having 1 to 6 carbon atoms. The terms “C₁₋₄haloalkoxy” and “C₁₋₃haloalkoxy” are to be construed accordingly. The haloalkoxy group can be monohaloalkoxy, dihaloalkoxy or polyhaloalkoxy including perhaloalkyl. A monohaloalkyoxy can have one iodo, bromo, chloro or fluoro within the alkyl moiety of the alkoxy group. Dihaloalkoxy and polyhaloalkoxy groups can have two or more of the same halo atoms or a combination of different halo groups within the alkyl moiety of the alkoxy group. Typically the polyhaloalkoxy group contains up to 13, or 12, or 11, or 10, or 9, or 8, or 7, or 6, or 5, or 4, or 3, or 2 halo groups. Non-limiting examples of C₁₋₆haloalkoxy include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, difluoropropoxy, dichloroethoxy and dichloropropoxy.

The term “aryl” refers to an aromatic carbocyclic single ring radical or two fused ring system radical containing 6 to 10 carbon atoms. Examples include phenyl and naphthyl.

The term “heteroaryl” refers to a 5- to 10-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono-or bi-cyclic. In some embodiments, the monocyclic heteroaryl is a 5- or 6-membered monocyclic heteroaryl. 5- or 6-Membered monocyclic heteroaryl includes, for example, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, and the like. Bi-cyclic heteroaryls include groups in which a monocyclic heteroaryl ring is fused to one or more aryl or heteroaryl rings. Non-limiting examples include indolyl, indazoyl, benzofuranyl, benzimidazolyl, and imidazo[1,2-a]pyridine.

The term “carbocyclic ring” refers to a 4- to 12-membered saturated or partially unsaturated hydrocarbon ring and may exist as a single ring, bicyclic ring (including fused, spiro or bridged carbocyclic rings) or a spiro ring.

The term “carbocyclyl” refers to a 4- to 12-membered saturated or partially unsaturated hydrocarbon ring radical and may exist as a single ring, bicyclic ring (including fused, spiro or bridged carbocyclic rings) or a spiro ring.

Bi-cyclic carbocyclyl groups include, e.g., unsaturated carbocyclic radicals fused to another unsaturated carbocyclic radical, cycloalkyl, or aryl, such as, for example, cyclohexyl, cyclohexenyl, 2,3-dihydroindenyl, indanyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring radical which is fully saturated (e.g., cyclopropyl, cyclobutyl, cyclopentyl, and cyclohexyl). The term “C₃₋₄ cycloalkyl” refers to a carbocyclic ring radical which is fully saturated (e.g., cyclopropyl, cyclobutyl).

The term “heterocycle” refers to a 4- to 12-membered saturated or partially unsaturated heterocyclic ring containing 1 to 4 heteroatoms independently selected from N, O, and S. A heterocycle group may be mono- or bicyclic ring (e.g., a bridged, fused, or spiro bicyclic ring).

The term “heterocyclyl” refers to a 4- to 12-membered saturated or partially unsaturated heterocyclic radical containing 1 to 4 heteroatoms independently selected from N, O, and S. A heterocyclyl group may be mono- or bicyclic (e.g., a bridged, fused, or spiro bicyclic ring).

Examples of monocyclic saturated or partially unsaturated heterocyclic radicals include, without limitation, piperidinyl, piperazinyl, tetrahydropyranyl, morpholinyl, and pyrrolidinyl. Bi-cyclic heterocyclyl groups include, e.g., saturated or partially unsaturated heterocyclic radicals fused to another saturated or partially unsaturated heterocyclic radical, cycloalkyl, aryl, or heteroaryl ring, such as, for example, indolinyl, indolin-2-onyl, 2,3-dihydro-1H-pyrrolopyridinyl, 6,7-dihydro-5H-pyrrolopyrazinyl, 2-oxo-2,3-dihydro-1H-benzo[d]imidazolyl, 1,4,5,6-tetrahydrocyclopenta[c]pyrazolyl, 4,5,6,7-tetrahydrothieno[2,3-c]pyridinyl, 5,6-dihydro-4H-cyclopenta[b]thiophenyl, and 4,7-dihydro-5H-thieno[2,3-c]pyranyl. In some embodiments, the heterocyclyl group is a 3 to 6 membered monocyclic heterocyclyl group. In some embodiments, the 3 to 6 membered monocyclic heterocyclyl group is oxetanyl and tetrahydropyranyl. In some embodiments, the heterocyclyl group is a 4 to 6 membered monocyclic heterocyclyl group. In some embodiments, the heterocyclyl group is a 8 to 10 membered bicyclic heterocyclyl group.

As used herein the term “spiro” ring means a two-ring system wherein both rings share one common atom. Examples of spiro rings include 5-oxaspiro[2.3]hexanyl, oxaspiro[2.4]heptanyl, 5-oxaspiro[2.4]heptanyl, 4-oxaspiro[2.4]heptanyl, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptanyl, 2,2,6-diazaspiro[3.3]heptanyl, 3-azaspiro[5.5]undecanyl, 3,9-diazaspiro[5.5]undecanyl, 7-azaspiro[3.5]nonanyl, 2,6-diazaspiro[3.4]octanyl, 8-azaspiro[4.5]decanyl, 1,6-diazaspiro[3.3]heptanyl, 5-azaspiro[2.5]octanyl, 4,7-diazaspiro[2.5]octanyl, 5-oxa-2-azaspiro[3.4]octanyl, 6-oxa-1-azaspiro[3.3]heptanyl, 3-azaspiro[5.5]undecanyl, 3,9-diazaspiro[5.5]undecanyl, and the like.

The term “fused” ring refers to two ring systems share two adjacent ring atoms. Fused heterocycles have at least one of the ring systems contain a ring atom that is a heteroatom selected from O, N and S (e.g., 3-oxabicyclo[3.1.0]hexane). The term “bridged C₅₋₁₂cycloalkyl” refers to a 5- to 12-membered bridged carbocyclic ring radical. Examples of bridged C₅₋₁₂cycloalkyl include, without limitation, bicyclo[1.1.1]pentanyl.

As used herein the term “bridged” refers to a 5 to 12 membered cyclic moiety connected at two non-adjacent ring atoms (e.g. bicyclo[1.1.1]pentane, bicyclo [2.2.1]heptane and bicyclo [3.2.1]octane). The term “bridged C₅₋₁₂cycloalkyl” refers to a 5- to 12-membered bridged carbocyclic ring radical. Examples of bridged C₅₋₁₂cycloalkyl include, without limitation, bicyclo[1.1.1]pentanyl.

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I) or (IA), as well as all stereoisomers (including diastereoisomers and enantiomers), rotamers, and tautomers. When a moiety is present that is capable of forming a salt, then salts are included as well, in particular pharmaceutically acceptable salts.

As used herein, the term “a,” “an,” “the” and similar terms used in the context of the present disclosure (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the disclosure and does not pose a limitation on the scope of the disclosure otherwise claimed.

It is also possible that the intermediates and compounds of the present disclosure may exist in different tautomeric forms, and all such forms are embraced within the scope of the disclosure. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) or (IA) as defined herein, in free form. In another embodiment, the present disclosure relates to a compound of the Formula (I) or (IA) as defined herein, in salt form. In another embodiment, the present disclosure relates to a compound of the Formula (I) or (IA) as defined herein, in acid addition salt form. In a further embodiment, the present disclosure relates to a compound of the Formula (I) or (IA) as defined herein, in pharmaceutically acceptable salt form. In yet a further embodiment, the present disclosure relates to a compound of the Formula (I) or (IA) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

### Exemplification

### Abbreviations

Cs₂CO₃=cesium carbonate
DCM=CH₂Cl₂=dichloromethane
DIPEA=DIEA=diisopropylethyl amine
DMF=dimethylformamide
DMSO=dimethylsulfoxide
Et₂O=diethyl ether
EtOAc=EA=ethyl acetate
EtOH=ethanol
h=hour
H₂O=water
H₂SO₄=sulfuric acid
HATU=Hexafluorophosphate Azabenzotriazole Tetramethyl Uronium
HCl=hydrochloric acid
HNO₃=nitric acid
HPLC=high pressure liquid chromatography
K₂CO₃=potassium carbonate
KI=potassium iodide
LCMS=liquid chromatography mass spectrometry
LDA=lthium diisopropylamide
LiAlH₄=LAH=lithium aluminium hydride
LiBH₄=Lithium borohydride
LiCl=lithium chloride
MeCN=ACN=CH₃CN=acetonitrile
MeOH=methanol
N₂=Nitrogen
Na₂SO₄=sodium sulfate
NaH=sodium hydride
NaOEt=sodium ethoxide
NaOH=Sodium Hydroxide
NaOMe=sodium methoxide
n-BuLi=n-butyl lithium
NH₄Cl=ammonium chloride
NH₄OH=ammonium hydroxide
NMR=Nuclear Magnetic Resonance Spectroscopy
NOESY=Nuclear Overhauser Effect Spectroscopy
Pd(PPh₃)₄=Tetrakis(triphenylphosphine)palladium(O)
Pd/C=Palladium on Carbon
Pd₂(dba)₃=Tris(dibenzylideneacetone)dipalladium(O)
POBr₃=phosphoryl bromide
POCl₃=phosphoryl chloride
PPA=polyphosphoric acid
RT=room temperature
t-BuOH=tert-butanol
t-BuOK=potassium tert-butoxide
TEA=Et₃N=triethylamine
TFA=trifluoroacetic acid
THF=tetrahydrofuran
TLC=Thin Layer Chromatography
Xantphos=4,5-Bis(diphenylphosphino)-9,9-dimethylxanthene

### Example 1: N-(4-(2-methoxyethoxy)-2-(thiazol-5-yl)quinolin-6-yl)oxetane-3-carboxamide

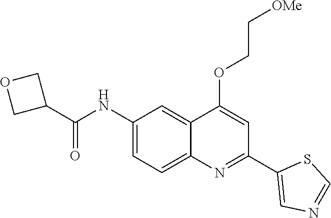 [SMILES: COCCOc1cc(-c2cncs2)nc2ccc(NC(=O)C3COC3)cc12]

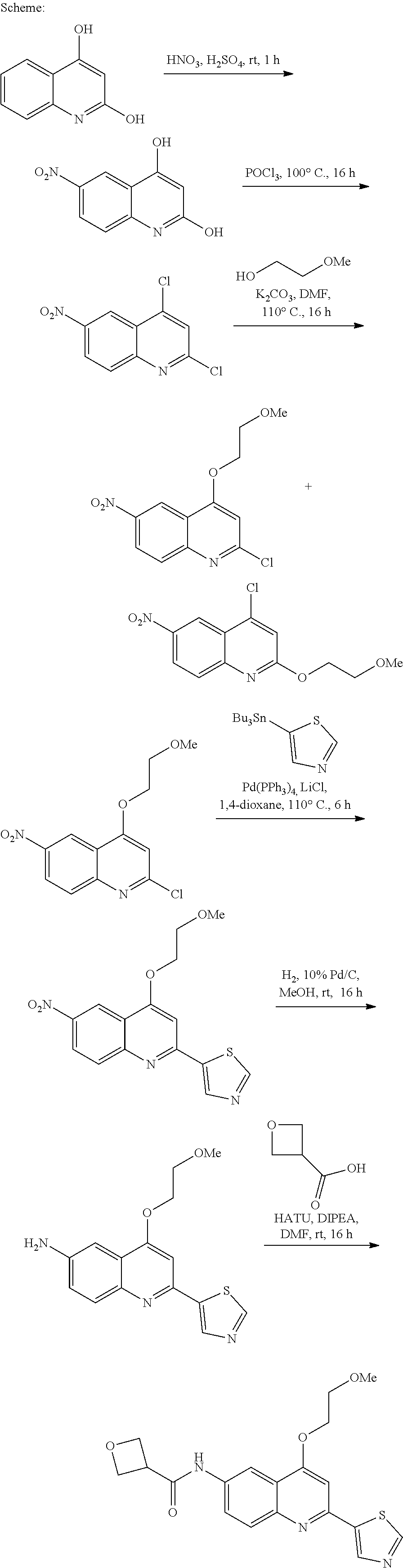

### 1. Synthesis of 6-nitroquinoline-2,4-diol

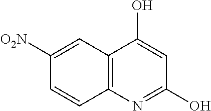 [SMILES: O=[N+]([O-])c1ccc2nc(O)cc(O)c2c1]

To a stirred solution of quinoline-2,4-diol (5 g, 31.03 mmol) in H₂SO₄ (45 mL) was added HNO₃ (1.2 mL) dropwise at RT. The mixture was stirred at RT for 30 min. Then, the reaction mixture was poured into ice cold water (200 mL). The formed precipitate was filtered and washed with water (50 mL), dried in vacuo to afford 6-nitroquinoline-2,4-diol (4.5 g, yield: 70%) as yellow solid. ¹H NMR (400 MHz, DMSO-d₆, δ): 11.48-12.14 (m, 2H), 8.56 (d, J=2.57 Hz, 1H), 8.32 (dd, J=9.05, 2.57 Hz, 1H), 7.39 (d, J=9.05 Hz, 1H), 5.83 (s, 1H).

### 2. Synthesis of 2,4-dichloro-6-nitroquinoline

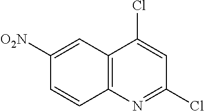 [SMILES: O=[N+]([O-])c1ccc2nc(Cl)cc(Cl)c2c1]

A stirred solution of 6-nitroquinoline-2,4-diol (10 g, 48.51 mmol) in POCl₃ (100 mL) was heated at 100° C. for 24 h. Then, the reaction mixture was cooled to RT and quenched slowly with ice cold water (100 mL). The precipitate formed was filtered and dried in vacuo to afford 2,4-dichloro-6-nitroquinoline (8.6 g, yield: 73%) as yellow solid. ¹H NMR (400 MHz, DMSO-d₆, δ): 8.96 (d, J=1.47 Hz, 1H), 8.58-8.66 (m, 1H), 8.16-8.34 (m, 2H).

### 3. Synthesis of 2-chloro-4-(2-methoxyethoxy)-6-nitroquinoline

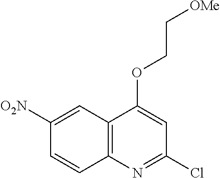 [SMILES: COCCOc1cc(Cl)nc2ccc([N+](=O)[O-])cc12]

To a stirred solution of 2,4-dichloro-6-nitroquinoline (2 g, 8.29 mmol) and 2-methoxyethan-1-ol (0.76 g, 9.95 mmol) in DMF (20 mL) was added K₂CO₃ (1.8 g, 12.43 mmol) at RT and heated at 110° C. for 16 h. Then, the reaction mixture was cooled to RT and poured into water (200 mL). The aqueous layer was extracted with EtOAc (2×200 mL). The combined organic layer was washed with brine (100 mL), dried over anhydrous Na₂SO₄, filtered and concentrated in vacuo to afford mixture of 2-chloro-4-(2-methoxyethoxy)-6-nitroquinoline and 4-chloro-2-(2-methoxyethoxy)-6-nitroquinoline. The crude obtained was purified by combi flash chromatography (dichloromethane) to afford 2-chloro-4-(2-methoxyethoxy)-6-nitroquinoline (0.4 g, yield: 17%) as an off white solid and 4-chloro-2-(2-methoxyethoxy)-6-nitroquinoline (0.8 g, yield: 35%) as an off-white solid. Note: The isomers were confirmed by 2D NOESY. 2-chloro-4-(2-methoxyethoxy)-6-nitroquinoline: LCMS (ESI+): m/z calcd. for C₁₂H₁₂ClN₂O₄[M+H]⁺, 283; found, 283. ¹H NMR (400 MHz, CHLOROFORM-d, δ): 9.12 (d, J=2.45 Hz, 1H), 8.48 (dd, J=9.29, 2.45 Hz, 1H), 8.05 (d, J=9.29 Hz, 1H), 6.90 (s, 1H), 4.42 (t, J=4.89 Hz, 2H), 3.93 (t, J=4.89 Hz, 2H) 3.51 (s, 3H). 4-chloro-2-(2-methoxyethoxy)-6-nitroquinoline: LCMS (ESI+): m/z calcd. for C₁₂H₁₂ClN₂O₄ [M+H]⁺, 283; found, 283. ¹H NMR (400 MHz, CHLOROFORM-d, δ): 9.05 (s, 1H), 8.44 (d, J=9.29 Hz, 1H), 7.92 (d, J=9.29 Hz, 1H), 7.24 (d, J=14.67 Hz, 1H), 4.69 (t, J=4.40 Hz, 2H), 3.81 (t, J=4.40 Hz, 2H), 3.48 (s, 3H).

### 4: Synthesis of 5-(4-(2-methoxyethoxy)-6-nitroquinolin-2-yl) thiazole

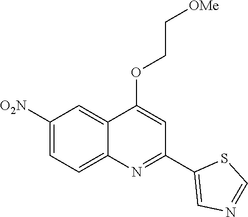 [SMILES: COCCOc1cc(-c2cncs2)nc2ccc([N+](=O)[O-])cc12]

A stirred solution of 2-chloro-4-(2-methoxyethoxy)-6-nitroquinoline (0.25 g, 0.88 mmol) and LiCl (0.11 g, 2.65 mmol) in 1,4-dioxane (3 mL) was purged with N₂ for 30 min. 5-(tributylstannyl)thiazole (0.50 g, 1.32 mmol) and Pd(PPh₃)₄(0.2 g, 0.17 mmol) were added and purged with N₂ for another 5 min. The reaction was heated at 110° C. for 16 h in a sealed tube. Then, the reaction mixture was cooled to RT and diluted with water (50 mL). The aqueous layer was extracted with EtOAc (2×100 mL). The combined organic layer was washed with brine (100 mL), dried over anhydrous Na₂SO₄, filtered and concentrated in vacuo. The crude obtained was triturated with CH₃CN (5 mL) and dried in vacuo to afford 5-(4-(2-methoxyethoxy)-6-nitroquinolin-2-yl) thiazole (0.24 g, yield: 82%) as white solid. The obtained compound was used in the next step without purification. LCMS (ESI+): m/z calcd. for C₁₅H₁₄N₃O₄S [M+H]⁺, 332; found, 332. ¹H NMR (400 MHz, DMSO-d₆, δ): 9.30 (s, 1H), 8.97 (s, 1H), 8.87 (d, J=2.45 Hz, 1H), 8.43 (dd, J=9.29, 2.93 Hz, 1H), 8.08 (d, J=9.29 Hz, 1H), 7.85 (s, 1H), 4.60 (t, J=3.42 Hz, 2H), 3.90 (t, J=3.91 Hz, 2H), 3.41 (s, 3H).

### 5. Synthesis of 4-(2-methoxyethoxy)-2-(thiazol-5-yl) quinolin-6-amine

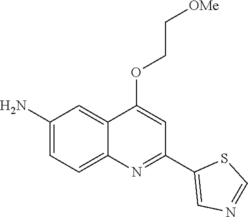 [SMILES: COCCOc1cc(-c2cncs2)nc2ccc(N)cc12]

To a stirred solution of 5-(4-(2-methoxyethoxy)-6-nitroquinolin-2-yl) thiazole (0.22 g, 0.4 mmol) in MeOH (5 mL) was added 10% Pd/C (50% wet, 30 mg) and the reaction was stirred under hydrogen atmosphere at 50 psi in a steel pressure vessel at RT for 16 h. Then, the reaction mixture was filtered through the pad of celite, washed with 10% MeOH in DCM (100 mL). The filtrate was concentrated in vacuo to afford 4-(2-methoxyethoxy)-2-(thiazol-5-yl) quinolin-6-amine (0.2 g, crude) as sticky black solid. The crude obtained compound was used in the next step without purification. LCMS (ESI+): m/z calcd. for C₁₅H₁₆N₃O₂S [M+H]⁺, 302; found, 302. ¹H NMR (400 MHz, DMSO-d₆, δ): 9.09 (s, 1H) 8.66 (s, 1H) 7.61 (d, J=8.80 Hz, 1H) 7.44 (s, 1H) 7.11 (dd, J=9.29, 2.45 Hz, 1H) 7.06 (d, J=2.45 Hz, 1H) 5.71 (br s, 2H) 4.43 (d, J=3.91 Hz, 2H) 3.83 (t, J=3.91 Hz, 2H) 3.39 (s, 3H).

### 6. Synthesis of N-(4-(2-methoxyethoxy)-2-(thiazol-5-yl)quinolin-6-yl)oxetane-3-carboxamide

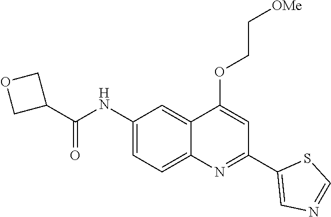 [SMILES: COCCOc1cc(-c2cncs2)nc2ccc(NC(=O)C3COC3)cc12]

To a stirred solution of oxetane-3-carboxylic acid (0.07 g, 0.72 mmol) in DMF (3 mL) was added HATU (0.44 g, 1.17 mmol) and stirred for 10 min. 4-(2-methoxyethoxy)-2-(thiazol-5-yl) quinolin-6-amine (0.18 g, 0.60 mmol) was added to the reaction mixture and followed by addition of DIPEA (0.28 mL, 1.56 mmol) at RT. The reaction was stirred at RT for 16 h. Then, the reaction mixture was diluted with water (50 mL) and extracted with 10% MeOH in DCM (2×100 mL). The organic layer was washed with brine (50 mL), dried over anhydrous Na₂SO₄, filtered and concentrated in vacuo. The crude obtained was purified by revers phase Preparative HPLC to afford N-(4-(2-methoxyethoxy)-2-(thiazol-5-yl)quinolin-6-yl)oxetane-3-carboxamide (0.07 g, yield: 30%) as white solid. HPLC purity: 95.11%. LCMS (ESI+): m/z calcd. for C₁₉H₂₀N₃O₄S [M+H]⁺, 386; found, 386. ¹H NMR (400 MHz, DMSO-d₄, δ): 10.30 (s, 1H), 9.18 (s, 1H), 8.80 (s, 1H), 8.45 (d, J=2.25 Hz, 1H), 7.97 (dd, J=9.13, 2.38 Hz, 1H), 7.87 (d, J=9.13 Hz, 1H), 7.63 (s, 1H), 4.74 (d, J=7.50 Hz, 4H), 4.53 (dd, J=5.32, 3.69 Hz, 2H), 4.02 (quin, J=7.54 Hz, 1H), 3.83-3.90 (m, 2H), 3.41 (s, 3H).

### Example 2: N-(4-ethoxy-2-(4-methyl-1H-imidazol-1-yl)quinolin-6-yl)oxetane-3-carboxamide

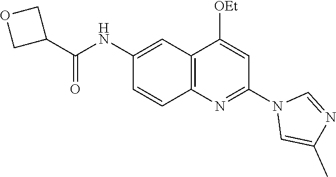 [SMILES: CCOc1cc(-n2cnc(C)c2)nc2ccc(NC(=O)C3COC3)cc12]

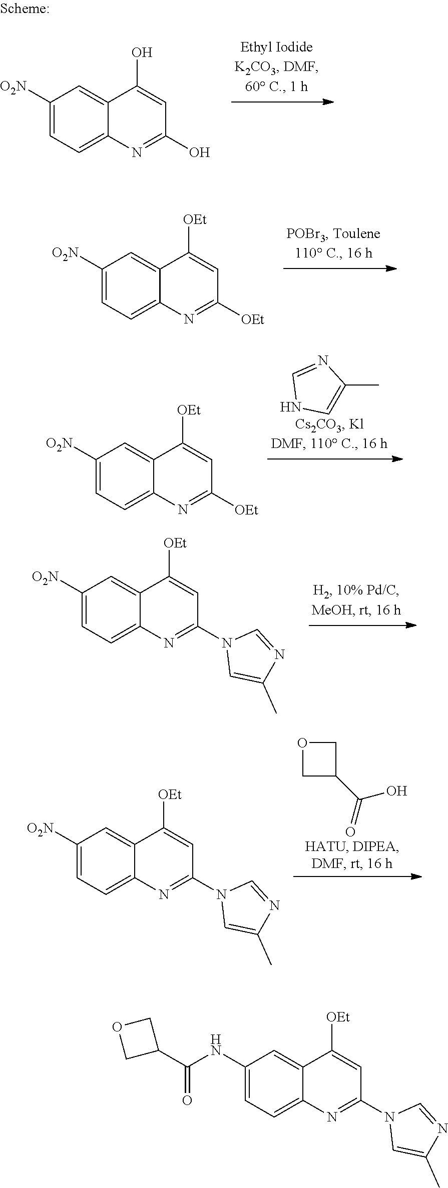

### 1. Synthesis of 4-ethoxy-6-nitroquinolin-2-ol

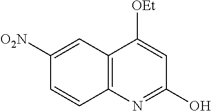 [SMILES: CCOc1cc(O)nc2ccc([N+](=O)[O-])cc12]

To a stirred solution of 6-nitroquinoline-2,4-diol (5 g, 24.26 mmol×2 batches) in DMF (100 mL) was added K₂CO₃ (6.7 g, 48.5 mmol) and heated to 60° C. for 1 h. The reaction was cooled to RT and ethyl iodide (3.7 g, 24.26 mmol) was added. The reaction mixture was stirred at RT for 16 h. Then, the reaction mixture was poured into ice cold water (500 mL), solid was filtered, washed with water (100 mL) and dried in vacuo. The crude obtained was triturated with 80% EtOAc in heptane to afford 4-ethoxy-6-nitroquinolin-2-ol (5 g, crude) as pale brown solid. ¹H NMR (400 MHz, METHANOL-d₄, δ): 12.71 (s, 1H), 9.32 (d, J=2.45 Hz, 1H), 9.13 (dd, J=9.29, 2.45 Hz, 1H), 8.20 (d, J=9.29 Hz, 1H), 6.82 (s, 1H), 5.02 (q, J=7.17 Hz, 2H), 2.26 (t, J=6.85 Hz, 3H).

### 2. Synthesis of 2-bromo-4-ethoxy-6-nitroquinoline

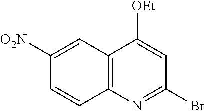 [SMILES: CCOc1cc(Br)nc2ccc([N+](=O)[O-])cc12]

To a stirred solution of 4-ethoxy-6-nitroquinolin-2-ol (5 g, 21.36 mmol) in toluene (50 mL) was added POBr₃ (18 g, 64.10 mmol) at RT and heated to 110° C. for 16 h. Then, the reaction mixture was quenched with ice cold water (200 mL), solid was filtered, washed with water (50 mL) and dried in vacuo. The crude obtained was purified by combi flash chromatography (70 to 80% EtOAc in heptane) to afford 2-bromo-4-ethoxy-6-nitroquinoline (3 g, yield: 47%) as pale yellow solid. LCMS (ESI+): m/z calcd. for C₁₁H₁₀BrN₂O₃[M+H]⁺, 298; found, 298. ¹H NMR (400 MHz, DMSO-d₄, δ): 8.88 (d, J=1.96 Hz, 1H), 8.49 (dd, J=9.29, 2.45 Hz, 1H), 8.08 (d, J=9.29 Hz, 1H), 7.43 (s, 1H), 4.44 (q, J=7.01 Hz, 2H), 1.51 (t, J=7.09 Hz, 3H).

### 3. Synthesis of 4-ethoxy-2-(4-methyl-1H-imidazol-1-yl)-6-nitroquinoline

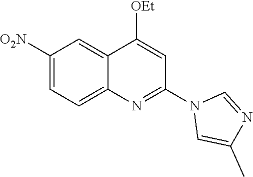 [SMILES: CCOc1cc(-n2cnc(C)c2)nc2ccc([N+](=O)[O-])cc12]

To a stirred solution of 4-methyl-1H-imidazole (0.1 g, 1.26 mmol) in DMF (10 mL) was added Cs₂CO₃ (0.82 g, 2.52 mmol) and KI (0.013 g, 0.08 mmol) at RT and stirred for 10 min and 2-bromo-4-ethoxy-6-nitroquinoline (0.25 g, 0.84 mmol) was added at RT and heated to 100° C. for 16 h. Then, the reaction mixture was cooled to RT and poured into ice water (100 mL), filtered the solid, washed with washed with water (20 mL), dried in vacuo. The crude obtained was triturated with n-pentane (10 mL) and Et₂O (10 mL), dried in vacuo to afford 4-ethoxy-2-(4-methyl-1H-imidazol-1-yl)-6-nitroquinoline (0.2 g, yield: 80%) as pale yellow solid. LCMS (ESI+): m/z calcd. for C₁₅H₁₅N₄O₃ [M+H]⁺, 299; found, 299. ¹H NMR (400 MHz, DMSO-d₆, δ): 8.84 (d, J=2.45 Hz, 1H), 8.70 (s, 1H), 8.45 (dd, J=9.29, 2.45 Hz, 1H), 7.99 (d, J=9.29 Hz, 1H), 7.89 (s, 1H), 7.49 (s, 1H), 4.50 (q, J=6.85 Hz, 2H), 2.21 (s, 3H), 1.55 (t, J=7.09 Hz, 3H).

### 4. Synthesis of 4-ethoxy-2-(4-methyl-1H-imidazol-1-yl) quinolin-6-amine

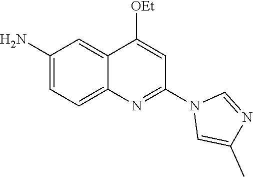 [SMILES: CCOc1cc(-n2cnc(C)c2)nc2ccc(N)cc12]

To a stirred solution of 4-ethoxy-2-(4-methyl-1H-imidazol-1-yl)-6-nitroquinoline (0.2 g, 0.67 mmol) in MeOH (10 mL) was added 10% Pd/C (50% wet, 0.07 g) and the reaction was stirred under hydrogen atmosphere at 50 psi in a steel pressure vessel at RT for 16 h. Then, the reaction mixture was filtered through the pad of Celite, washed with MeOH (50 mL). The filtrate was concentrated in vacuo to afford 4-ethoxy-2-(4-methyl-1H-imidazol-1-yl) quinolin-6-amine (0.15 g, crude) as pale yellow solid. The crude obtained compound was used in the next step without purification. LCMS (ESI+): m/z calcd. for C₁₅H₁₇N₄O [M+H]⁺, 269; found, 269. ¹H NMR (400 MHz, DMSO-d₆, δ): 8.47 (s, 1H), 7.74 (s, 1H), 7.56 (d, J=8.80 Hz, 1H), 7.10-7.17 (m, 2H), 7.07 (s, 1H), 5.59 (s, 2H), 4.36 (q, J=6.85 Hz, 2H), 2.18 (s, 3H), 1.48 (t, J=6.85 Hz, 3H).

### 5. Synthesis N-(4-ethoxy-2-(4-methyl-1H-imidazol-1-yl)quinolin-6-yl)oxetane-3-carboxamide

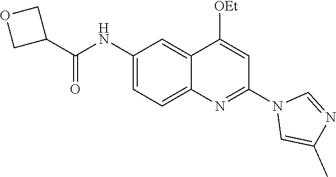 [SMILES: CCOc1cc(-n2cnc(C)c2)nc2ccc(NC(=O)C3COC3)cc12]

To a stirred solution of oxetane-3-carboxylic acid (0.085 g, 0.83 mmol) in DMF (10 mL) was added HATU (0.53 g, 1.39 mmol) and stirred for 10 min. 4-Ethoxy-2-(4-methyl-1H-imidazol-1-yl) quinolin-6-amine (0.15 g, 0.55 mmol) was added to the reaction mixture and followed by addition of DIPEA (0.3 mL, 1.67 mmol) at RT. The reaction mixture was stirred at RT for 16 h. Then, the reaction mixture was diluted with ice cold water (50 mL) and extracted with 5% MeOH in DCM (2×100 mL). The organic layer was washed with brine (50 mL), dried over anhydrous Na₂SO₄, filtered and concentrated in vacuo. The crude obtained was purified by combi flash chromatography (0 to 5% MeOH in DCM) to afford N-(4-ethoxy-2-(4-methyl-1H-imidazol-1-yl)quinolin-6-yl)oxetane-3-carboxamide (0.071 g, yield: 36%) as an off white solid. HPLC purity: 95.19%. LCMS (ESI+): m/z calcd. for C₁₉H₂₁N₄O₃ [M+H]⁺, 353; found, 353. ¹H NMR (400 MHz, DMSO-d₆, δ): 10.26 (s, 1H), 8.58 (d, J=1.00 Hz, 1H), 8.49 (d, J=2.25 Hz, 1H), 7.93 (dd, J=9.13, 2.38 Hz, 1H), 7.77-7.85 (m, 2H), 7.32 (s, 1H), 4.73 (d, J=7.50 Hz, 4H), 4.44 (q, J=7.00 Hz, 2H), 4.01 (quin, J=7.47 Hz, 1H), 2.20 (s, 3H), 1.52 (t, J=6.94 Hz, 3H).

### Example 3: N-(4-ethoxy-2-(thiazol-5-yl)quinolin-6-yl)oxetane-3-carboxamide

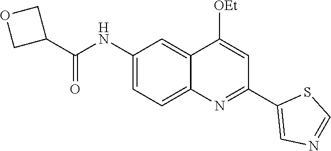 [SMILES: CCOc1cc(-c2cncs2)nc2ccc(NC(=O)C3COC3)cc12]

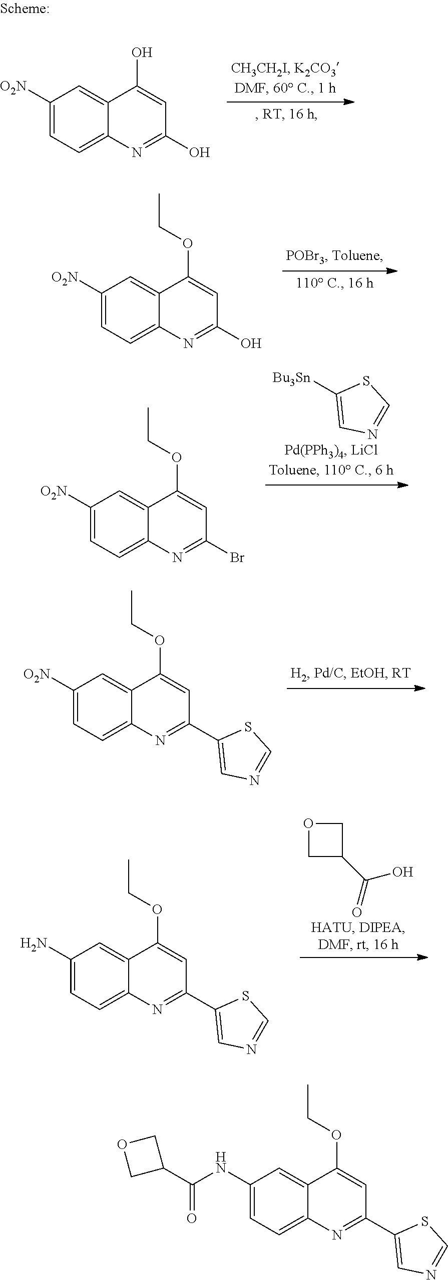

### 1. Synthesis of 5-(4-ethoxy-6-nitroquinolin-2-yl) thiazole

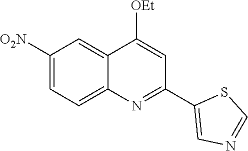 [SMILES: CCOc1cc(-c2cncs2)nc2ccc([N+](=O)[O-])cc12]

5-(4-Ethoxy-6-nitroquinolin-2-yl) thiazole was prepared using the procedure followed for preparation of 5-(4-(2-methoxyethoxy)-6-nitroquinolin-2-yl) thiazole. The obtained compound was purified by combi flash chromatography (5 to 10% EtOAc in heptane) to give a yellow solid (0.4 g, yield: 65%). LCMS (ESI+): m/z calcd. for C₁₄H₁₂N₃O₃S [M+H]⁺, 302; found, 302. ¹H NMR (400 MHz, DMSO-d₆, δ): 9.29 (s, 1H), 8.98 (s, 1H), 8.89 (d, J=2.45 Hz, 1H), 8.43 (dd, J=9.29, 2.45 Hz, 1H), 8.08 (d, J=9.29 Hz, 1H), 7.81 (s, 1H), 4.52 (q, J=7.01 Hz, 2H), 1.56 (s, 3H).

### 2. Synthesis of 4-ethoxy-2-(thiazol-5-yl) quinolin-6-amine

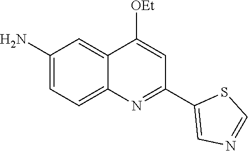 [SMILES: CCOc1cc(-c2cncs2)nc2ccc(N)cc12]

4-Ethoxy-2-(thiazol-5-yl) quinolin-6-amine was prepared using the procedure followed for preparation of 4-(2-methoxyethoxy)-2-(thiazol-5-yl) quinolin-6-amine as a black solid (0.21 g, crude), and was used for the next step without purification. LCMS (ESI+): m/z calcd. for C₁₄H₁₄N₃OS [M+H]⁺, 272; found, 272. ¹H NMR (400 MHz, DMSO-d₆, δ): 9.08 (s, 1H), 8.65 (s, 1H), 7.58-7.62 (m, 1H), 7.40 (s, 1H), 7.08-7.12 (m, 1H), 7.04-7.07 (m, 1H), 5.67 (s, 2H), 4.35 (q, J=7.15 Hz, 2H), 1.49 (t, J=7.15 Hz, 3H).

### 3. Synthesis of N-(4-ethoxy-2-(thiazol-5-yl)quinolin-6-yl)oxetane-3-carboxamide

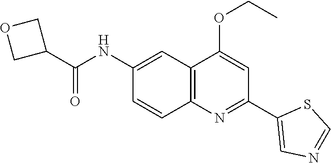 [SMILES: CCOc1cc(-c2cncs2)nc2ccc(NC(=O)C3COC3)cc12]

N-(4-ethoxy-2-(thiazol-5-yl)quinolin-6-yl)oxetane-3-carboxamide was prepared by replacing 4-ethoxy-2-(thiazol-5-yl) quinolin-6-amine for 4-(2-methoxyethoxy)-2-(thiazol-5-yl) quinolin-6-amine in N-(4-(2-methoxyethoxy)-2-(thiazol-5-yl)quinolin-6-yl)oxetane-3-carboxamide to yield an off white solid (0.04 g, yield: 31%). HPLC purity: 98.93%. LCMS (ESI+): m/z calcd. for C₁₈H₁₈N₃O₃S [M+H]⁺, 356; found, 356. ¹H NMR (400 MHz, DMSO-d₆, δ): 10.27 (s, 1H), 9.17 (s, 1H), 8.80 (s, 1H), 8.49 (d, J=2.00 Hz, 1H), 7.82-7.96 (m, 2H), 7.60 (s, 1H), 4.74 (d, J=7.38 Hz, 4H), 4.44 (d, J=7.00 Hz, 2H), 4.01 (t, J=7.50 Hz, 1H), 1.53 (t, J=6.94 Hz, 3H).

### Example 4: N-(4-ethoxy-2-(thiazol-5-yl)quinolin-6-yl)bicyclo[1.1.1]pentane-1-carboxamide

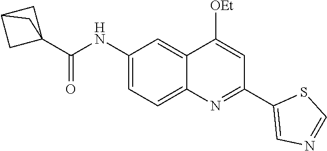 [SMILES: CCOc1cc(-c2cncs2)nc2ccc(NC(=O)C34CC(C3)C4)cc12]

N-(4-ethoxy-2-(thiazol-5-yl)quinolin-6-yl)bicyclo[1.1.1]pentane-1-carboxamide was prepared by replacing bicyclo[1.1.1]pentane-1-carboxylic acid for oxetane-3-carboxylic acid in N-(4-(2-methoxyethoxy)-2-(thiazol-5-yl)quinolin-6-yl)oxetane-3-carboxamide to yield an off white solid (0.05 g, yield: 46%). HPLC purity: 95.22%. LCMS (ESI+): m/z calcd. for C₂₀H₂₀N₃O₂S [M+H]⁺, 366; found, 366. ¹H NMR (400 MHz, DMSO-d₆, S): 9.86 (s, 1H), 9.17 (s, 1H), 8.80 (s, 1H), 8.43 (d, J=2.25 Hz, 1H), 8.06 (dd, J=9.13, 2.38 Hz, 1H), 7.84 (d, J=9.13 Hz, 1H), 7.58 (s, 1H), 4.44 (q, J=6.96 Hz, 2H), 2.51-2.49 (m, 1H), 2.12 (s, 6H), 1.52 (t, J=7.00 Hz, 3H).

### Example 5: N-(4-methoxy-2-(thiazol-5-yl) quinolin-6-yl) oxetane-3-carboxamide

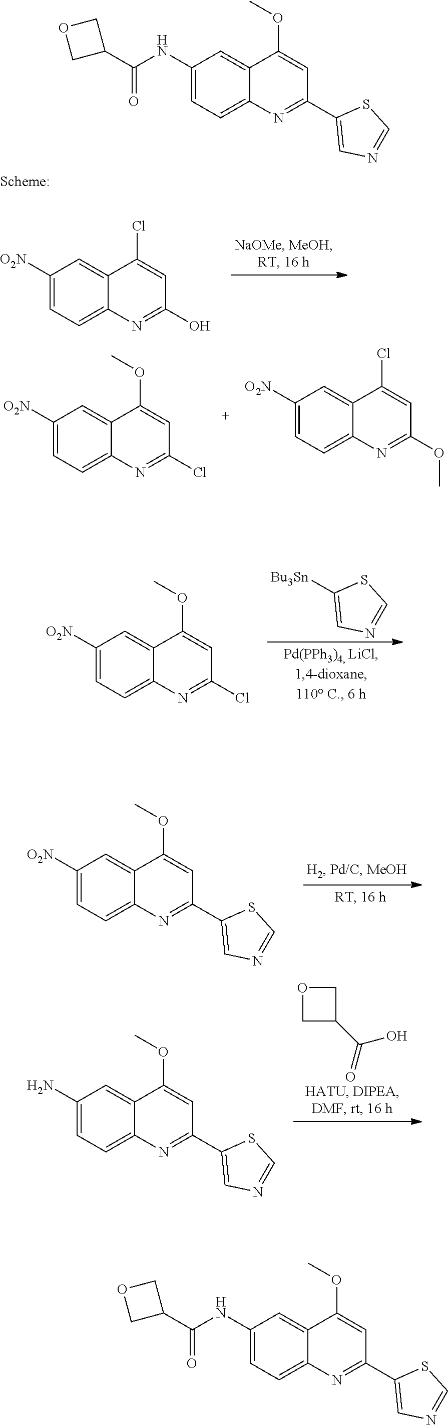

### 1. Synthesis of 2-chloro-4-methoxy-6-nitroquinoline

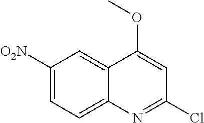 [SMILES: COc1cc(Cl)nc2ccc([N+](=O)[O-])cc12]

A stirred solution of 2,4-dichloro-6-nitroquinoline (2 g, 10.64 mmol) in MeOH (20 mL) was added NaOMe (0.42 g, 31.02 mmol) at RT. The reaction mixture was stirred at RT and for 16 h. Then, the volatiles were removed in vacuo. The crude was diluted with water (50 mL). The aqueous layer was extracted with EtOAc (2×75 mL). The combined organic layer was washed with brine (100 mL), dried over anhydrous Na₂SO₄, filtered and concentrated in vacuo to afford mixture of the 2-chloro-4-methoxy-6-nitroquinoline and 4-chloro-2-methoxy-6-nitroquinoline. The crude obtained was purified by combi flash chromatography (40 to 50% EtOAc in heptane) to afford 2-chloro-4-methoxy-6-nitroquinoline (0.32 g, yield: 31%) as an off white solid and 4-chloro-2-methoxy-6-nitroquinoline (0.6 g, yield: 35%) as an off-white solid. Note: The isomers were confirmed by 2D NOESY. 2-chloro-4-methoxy-6-nitroquinoline: LCMS (ESI+): m/z calcd. for C₁₀H₈ClN₂O₃[M+H]⁺, 239; found, 239. ¹H NMR (400 MHz, DMSO-d₆, δ): 8.87 (d, J=2.45 Hz, 1H), 8.49 (dd, J=9.29, 2.45 Hz, 1H), 8.07 (d, J=8.80 Hz, 1H), 7.34 (s, 1H), 4.16 (s, 3H). 4-chloro-2-methoxy-6-nitroquinoline: LCMS (ESI+): m/z calcd. for C₁₀H₈ClN₂O₃[M+H]⁺, 239; found, 239. ¹H NMR (400 MHz, DMSO-d₆, δ): 8.87 (d, J=2.45 Hz, 1H), 8.50 (dd, J=8.80, 2.45 Hz, 1H), 8.03 (d, J=9.29 Hz, 1H), 7.59 (s, 1H), 4.07 (s, 3H).

### 2. Synthesis of 5-(4-methoxy-6-nitroquinolin-2-yl) thiazole

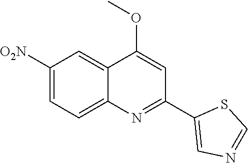 [SMILES: COc1cc(-c2cncs2)nc2ccc([N+](=O)[O-])cc12]

5-(4-Methoxy-6-nitroquinolin-2-yl) thiazole was prepared replacing 2-chloro-4-methoxy-6-nitroquinoline for 2-chloro-4-(2-methoxyethoxy)-6-nitroquinoline in the procedure for the synthesis of 5-(4-(2-methoxyethoxy)-6-nitroquinolin-2-yl) thiazole. The obtained compound was purified by combi flash chromatography (40 to 50% EtOAc in heptane) to give the product as a pale-yellow solid (0.22 g, yield: 57%). LCMS (ESI+): m/z calcd. for C₁₃H₁₀N₃O₃S [M+H]⁺, 288; found, 288. ¹H NMR (400 MHz, DMSO-d₆, S): 9.30 (s, 1H), 9.00 (s, 1H), 8.90 (d, J=2.45 Hz, 1H), 8.44 (dd, J=9.05, 2.69 Hz, 1H), 8.09 (d, J=9.29 Hz, 1H), 7.85 (s, 1H), 4.24 (s, 3H).

### 3. Synthesis of 4-methoxy-2-(thiazol-5-yl) quinolin-6-amine

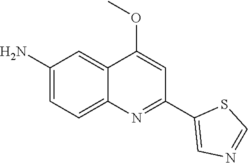 [SMILES: COc1cc(-c2cncs2)nc2ccc(N)cc12]

4-Methoxy-2-(thiazol-5-yl) quinolin-6-amine was prepared analogously as described for 4-(2-methoxyethoxy)-2-(thiazol-5-yl) quinolin-6-amine. The obtained compound was triturated with diethyl ether and n-pentane to give product as a pale yellow solid (0.15 g, yield: 76%). LCMS (ESI+): m/z calcd. for C₁₃H₁₂N₃OS [M+H]⁺, 258; found, 258. ¹H NMR (400 MHz, DMSO-d₆, δ): 9.09 (s, 1H), 8.67 (s, 1H), 7.61 (d, J=9.29 Hz, 1H), 7.42 (s, 1H), 7.10 (dd, J=8.80, 2.45 Hz, 1H), 7.04 (d, J=2.45 Hz, 1H), 5.67 (s, 2H), 4.07 (s, 3H).

### 4. Synthesis of N-(4-methoxy-2-(thiazol-5-yl) quinolin-6-yl) oxetane-3-carboxamide

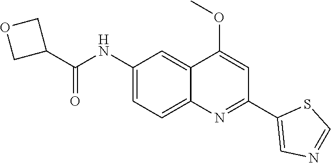 [SMILES: COc1cc(-c2cncs2)nc2ccc(NC(=O)C3COC3)cc12]

N-(4-methoxy-2-(thiazol-5-yl) quinolin-6-yl) oxetane-3-carboxamide was prepared using similar procedure to N-(4-(2-methoxyethoxy)-2-(thiazol-5-yl)quinolin-6-yl)oxetane-3-carboxamide. The crude was purified by combi flash column chromatography (5% MeOH/CH₂Cl₂) to give product as an off-white solid (0.09 g, yield: 45%). HPLC purity: 97.95%. LCMS (ESI+): m/z calcd. for C₁₇H₁₆N₃O₃S [M+H]⁺, 342; found, 342. ¹H NMR (400 MHz, DMSO-d₆, δ) 10.27 (s, 1H), 9.18 (s, 1H), 8.82 (s, 1H), 8.59 (d, J=2.13 Hz, 1H), 7.87 (d, J=9.01 Hz, 1H), 7.82 (t, J=2.25 Hz, 1H), 7.62 (s, 1H), 4.75-4.72 (m, 4H), 4.16 (s, 3H), 4.06-3.97 (m, 1H).

### Example 6: N-(4-ethoxy-2-(2-methylthiazol-5-yl) quinolin-6-yl) oxetane-3-carboxamide

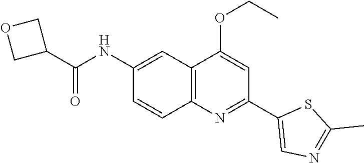 [SMILES: CCOc1cc(-c2cnc(C)s2)nc2ccc(NC(=O)C3COC3)cc12]

N-(4-ethoxy-2-(2-methylthiazol-5-yl) quinolin-6-yl) oxetane-3-carboxamide was prepared using similar procedure to Example 3 as an off white solid (0.05 g, yield: 50%). HPLC purity: 98.23%. LCMS (ESI+): m/z calcd. for C₁₉H₂₀N₃O₃S [M+H]⁺, 370; found, 370. ¹H NMR (400 MHz, DMSO-d₆, δ): 10.27 (s, 1H), 8.53 (s, 1H), 8.48 (d, J=2.25 Hz, 1H), 7.88-7.93 (m, 1H), 7.83 (d, J=9.13 Hz, 1H), 7.53 (s, 1H), 4.74 (d, J=7.50 Hz, 4H), 4.43 (q, J=7.00 Hz, 2H), 4.02 (quin, J=7.50 Hz, 1H), 2.70 (s, 3H), 1.53 (t, J=7.00 Hz, 3H).

### Example 7: N-(4-ethoxy-2-(2-methoxythiazol-5-yl) quinolin-6-yl) oxetane-3-carboxamide

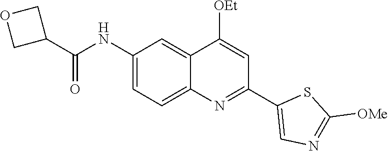 [SMILES: CCOc1cc(-c2cnc(OC)s2)nc2ccc(NC(=O)C3COC3)cc12]

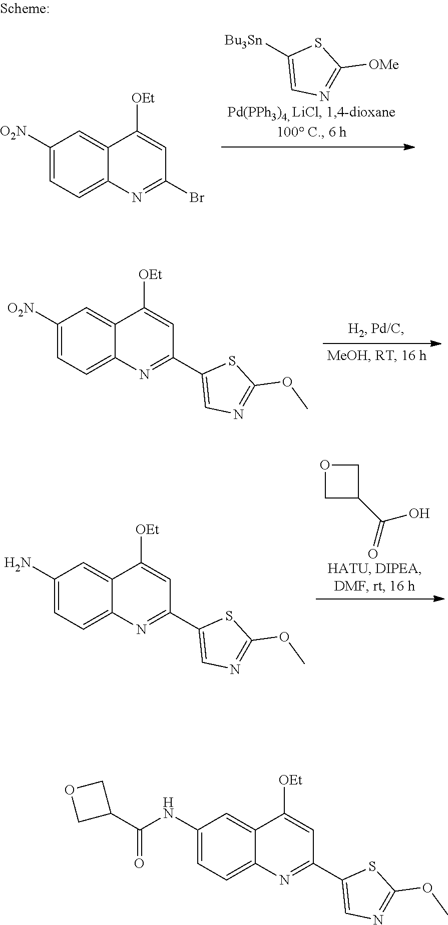

### 1. Synthesis of 5-(4-ethoxy-6-nitroquinolin-2-yl)-2-methoxythiazole

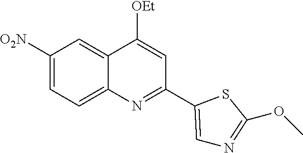 [SMILES: CCOc1cc(-c2cnc(OC)s2)nc2ccc([N+](=O)[O-])cc12]

5-(4-Ethoxy-6-nitroquinolin-2-yl)-2-methoxythiazole was prepared using the procedure for 5-(4-(2-methoxyethoxy)-6-nitroquinolin-2-yl) thiazole. The crude was purified by trituration with diethyl ether (25 mL) and n-pentane (25 mL) to give product as a pale-yellow solid (0.16 g, yield: 76%). LCMS (ESI+): m/z calcd. for C₁₅H₁₄N₃O₄S [M+H]⁺, 332; found, 332. ¹H NMR (400 MHz, DMSO-d₆, δ): 8.88 (d, J=2.45 Hz, 1H), 8.42 (dd, J=9.29, 2.93 Hz, 1H), 8.39 (s, 1H), 8.01 (d, J=9.29 Hz, 1H), 7.72 (s, 1H), 4.50 (q, J=7.01 Hz, 2H), 4.12 (s, 3H), 1.55 (t, J=6.85 Hz, 3H).

### 2. Synthesis of 4-ethoxy-2-(2-methoxythiazol-5-yl)quinolin-6-amine

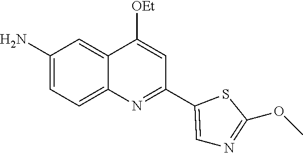 [SMILES: CCOc1cc(-c2cnc(OC)s2)nc2ccc(N)cc12]

4-Ethoxy-2-(2-methoxythiazol-5-yl)quinolin-6-amine was prepared using the procedure followed for preparation of 4-(2-methoxyethoxy)-2-(thiazol-5-yl) quinolin-6-amine as a pale-yellow sticky solid (0.11 g, 85%). LCMS (ESI+): m/z calcd. for C₁₅H₁₆N₃O₂S [M+H]⁺, 302; found, 302. ¹H NMR (400 MHz, DMSO-d₆, δ): 7.98 (s, 1H), 7.53 (d, J=8.80 Hz, 1H), 7.28 (s, 1H), 7.08 (t, J=3.2 Hz, 2H), 5.56 (br s, 2H), 4.32 (q, J=7.2 Hz, 2H), 4.05 (s, 3H), 1.47 (t, J=7.2 Hz, 3H).

### 3. Synthesis of N-(4-ethoxy-2-(2-methoxythiazol-5-yl) quinolin-6-yl) oxetane-3-carboxamide

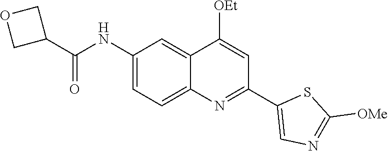 [SMILES: CCOc1cc(-c2cnc(OC)s2)nc2ccc(NC(=O)C3COC3)cc12]

N-(4-ethoxy-2-(2-methoxythiazol-5-yl) quinolin-6-yl) oxetane-3-carboxamide was prepared using similar procedure to N-(4-(2-methoxyethoxy)-2-(thiazol-5-yl)quinolin-6-yl)oxetane-3-carboxamide). The crude was purified by combi flash chromatography (1% MeOH/CH₂Cl₂) to give product as an off white solid (0.04 g, yield: 26%). HPLC purity: 97.09%. LCMS (ESI+): m/z calcd. for C₁₉H₂₀N₃O₄S [M+H]⁺, 386; found, 386. ¹H NMR (400 MHz, DMSO-d₆, δ): 10.24 (s, 1H), 8.45 (s, 1H), 8.14 (s, 1H), 7.88 (d, J=7.6 Hz, 1H), 7.78 (d, J=9.2 Hz, 1H), 7.47 (s, 1H), 4.71-4.74 (m, 4H), 4.40 (q, J=6.92 Hz, 2H), 4.08 (s 3H), 4.05-3.98 (m, 1H), 1.51 (t, J=6.94 Hz, 3H).

### Example 8: N-(2-(2-cyclopropylthiazol-5-yl)-4-ethoxyquinolin-6-yl) oxetane-3-carboxamide

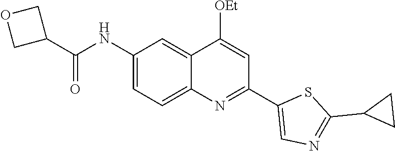 [SMILES: CCOc1cc(-c2cnc(C3CC3)s2)nc2ccc(NC(=O)C3COC3)cc12]

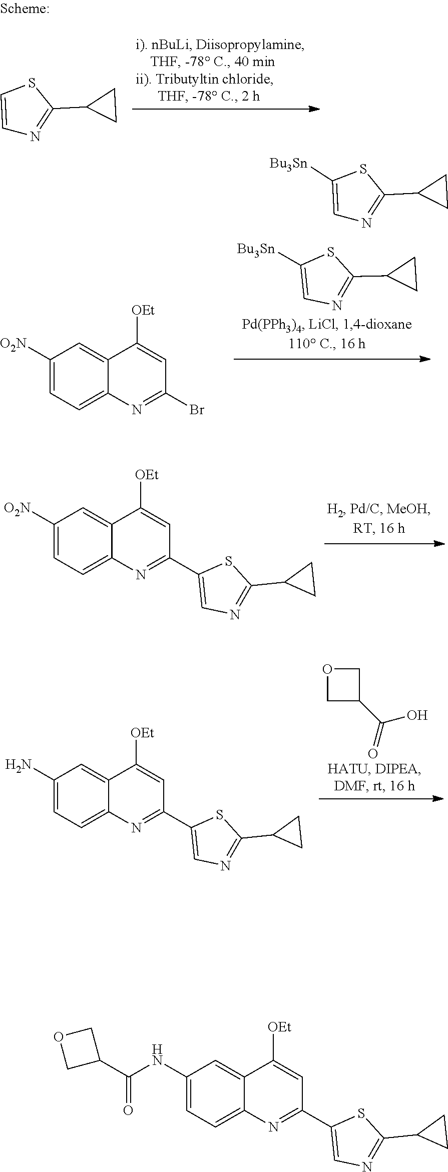

### 1. Synthesis of 2-cyclopropyl-5-(tributylstannyl) thiazole

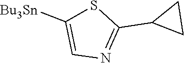

To a stirred solution of DIPEA (0.7 mL, 4.80 mmol) in THF (5 mL) was added n-BuLi (2.5 M sol. in hexanes, 1.5 mL, 3.84 mmol) dropwise for 5 min at −78° C. under argon atmosphere and stirred for 40 min. To a stirred solution of 2-cyclopropylthiazole (0.4 g, 3.20 mmol) in anhydrous THF (5 mL) was added freshly generated LDA solution dropwise for 5 min at−78° C. and stirred for 40 min at the same temperature. To this was added −78° C. for 1 h. To this was added tributyltin chloride (0.9 mL, 3.2 mmol) dropwise for 5 min at −78° C. and stirred at the same temperature for 2 h. The reaction mixture was quenched with saturated ammonium chloride solution (10 mL) and extracted with EtOAc (2×50 mL). The combined organic extracts were dried over sodium sulfate, filtered and concentrated in vacuo to afford crude. The crude obtained was purified by combi flash chromatography (100% heptane) to afford 2-cyclopropyl-5-(tributylstannyl) thiazole (0.2 g, yield: 18%) as a colorless oil.

### 2. Synthesis of 2-cyclopropyl-5-(4-ethoxy-6-nitroquinolin-2-yl) thiazole

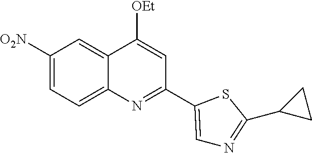 [SMILES: CCOc1cc(-c2cnc(C3CC3)s2)nc2ccc([N+](=O)[O-])cc12]

2-Cyclopropyl-5-(4-ethoxy-6-nitroquinolin-2-yl) thiazole was prepared using the procedure for 5-(4-(2-methoxyethoxy)-6-nitroquinolin-2-yl) thiazole. The obtained compound was purified by combi flash chromatography (20% EtOAc/heptane) to give product as an off-white solid (0.1 g, yield: 48%). HPLC: 98.1%. ¹H NMR (400 MHz, DMSO-d₆, δ): 8.88 (d, J=2.93 Hz, 1H), 8.67 (s, 1H), 8.42 (dd, J=9.29, 2.45 Hz, 1H), 8.03 (d, J=9.29 Hz, 1H), 7.74 (s, 1H), 4.50 (q, J=6.85 Hz, 2H), 1.55 (t, J=7.09 Hz, 3H), 1.16-1.27 (m, 3H), 1.02-1.11 (m, 2H).

### 3. Synthesis of 2-(2-cyclopropylthiazol-5-yl)-4-ethoxyquinolin-6-amine

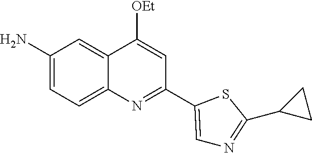 [SMILES: CCOc1cc(-c2cnc(C3CC3)s2)nc2ccc(N)cc12]

2-(2-Cyclopropylthiazol-5-yl)-4-ethoxyquinolin-6-amine was prepared using the procedure followed for preparation of 4-(2-methoxyethoxy)-2-(thiazol-5-yl) quinolin-6-amine as a pale-yellow sticky solid (0.11 g, crude). LCMS (ESI+): m/z calcd. for C₁₇H₁₈N₃OS [M+H]⁺, 312; found, 312.

### 4. Synthesis of N-(2-(2-cyclopropylthiazol-5-yl)-4-ethoxyquinolin-6-yl) oxetane-3-carboxamide

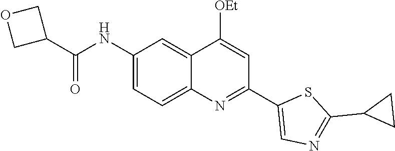 [SMILES: CCOc1cc(-c2cnc(C3CC3)s2)nc2ccc(NC(=O)C3COC3)cc12]

N-(2-(2-cyclopropylthiazol-5-yl)-4-ethoxyquinolin-6-yl) oxetane-3-carboxamide was prepared using similar procedure to N-(4-(2-methoxyethoxy)-2-(thiazol-5-yl)quinolin-6-yl)oxetane-3-carboxamide as an off-white solid (0.008 g, yield: 7%). HPLC purity: 96.44%. LCMS (ESI+): m/z calcd. for C₂₁H₂₂N₃O₃S [M+H]⁺, 396; found, 396. ¹H NMR (400 MHz, DMSO-d₆, δ): 10.25 (s, 1H), 8.44-8.50 (m, 2H), 7.88-7.92 (m, 1H), 7.77-7.84 (m, 1H), 7.50 (s, 1H), 4.75-4.71 (m, 4H), 4.41 (q, J=6.92 Hz, 2H), 4.06-3.96 (m, 1H), 2.38-2.48 (m, 1H), 1.51 (t, J=6.94 Hz, 3H), 1.13-1.22 (m, 2H), 0.99-1.07 (m, 2H).

### Example 9: N-(4-(2-hydroxyethoxy)-2-(thiazol-5-yl) quinolin-6-yl) oxetane-3-carboxamide

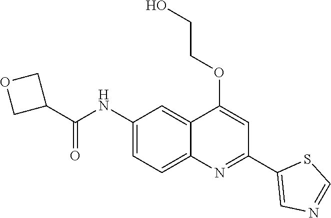 [SMILES: O=C(Nc1ccc2nc(-c3cncs3)cc(OCCO)c2c1)C1COC1]

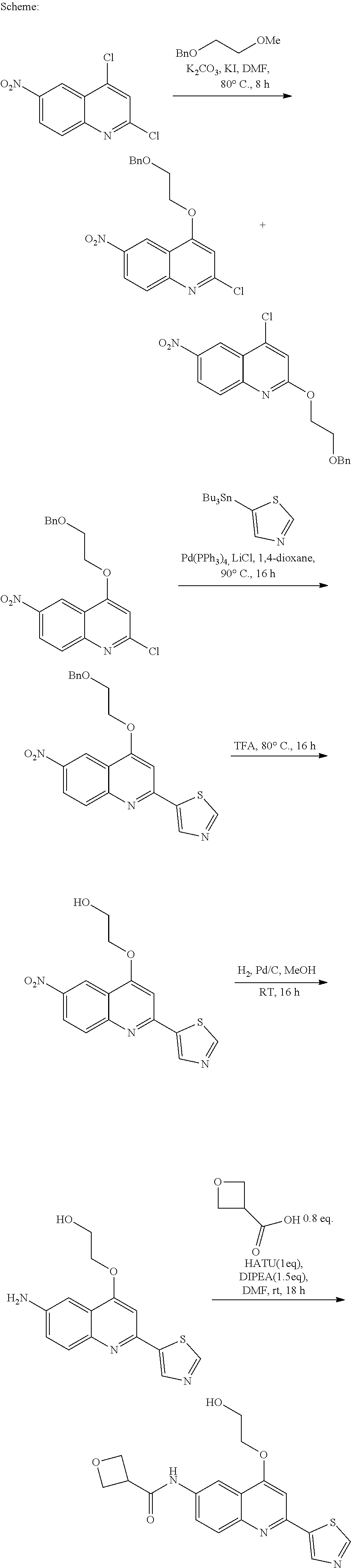

### 1. Synthesis of 4-(2-(benzyloxy)ethoxy)-2-chloro-6-nitroquinoline

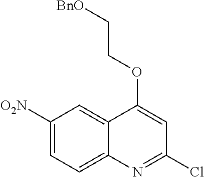 [SMILES: O=[N+]([O-])c1ccc2nc(Cl)cc(OCCOCc3ccccc3)c2c1]

4-(2-(Benzyloxy)ethoxy)-2-chloro-6-nitroquinoline was prepared using the procedure followed for preparation of 2-chloro-4-(2-methoxyethoxy)-6-nitroquinoline. The mixture were purified by combi flash chromatography (50 to 80% EtOAc in heptane) to afford 4-(2-(benzyloxy)ethoxy)-2-chloro-6-nitroquinoline (0.9 g, yield: 20%) as an off white solid and 2-(2-(benzyloxy)ethoxy)-4-chloro-6-nitroquinoline (0.9 g, yield: 20%) as an off-white solid. Note: The isomers were confirmed by 2D NOESY. 4-(2-(benzyloxy)ethoxy)-2-chloro-6-nitroquinoline: LCMS (ESI+): m/z calcd. for C₁₈H₁₆ClN₂O₄[M+H]⁺, 359; found, 359. ¹H NMR (400 MHz, DMSO-d₆, δ): 8.92 (d, J=2.63 Hz, 1H), 8.51 (dd, J=9.26, 2.63 Hz, 1H), 8.09 (d, J=9.26 Hz, 1H), 7.32-7.42 (m, 5H), 7.24-7.31 (m, 1H), 4.64 (s, 2H), 4.55-4.61 (m, 2H), 3.92-3.98 (m, 2H).

### 2. Synthesis of 5-(4-(2-(benzyloxy)ethoxy)-6-nitroquinolin-2-yl)thiazole

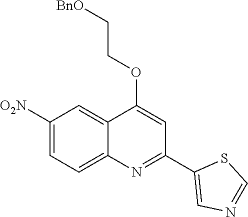 [SMILES: O=[N+]([O-])c1ccc2nc(-c3cncs3)cc(OCCOCc3ccccc3)c2c1]

5-(4-(2-(Benzyloxy)ethoxy)-6-nitroquinolin-2-yl)thiazole was prepared using the procedure for preparation of 5-(4-(2-methoxyethoxy)-6-nitroquinolin-2-yl) thiazole. The obtained compound was purified by combi flash chromatography (1% MeOH in CH₂Cl₂) to give the product as a pale brown solid (0.38 g, yield: 74%). LCMS (ESI+): m/z calcd. for C₂₁H₁₈N₃O₄S [M+H]⁺, 408; found, 408. ¹H NMR (400 MHz, DMSO-d₆, δ): 9.29 (s, 1H), 8.96 (s, 1H), 8.92 (d, J=2.45 Hz, 1H), 8.45 (dd, J=9.29, 2.93 Hz, 1H), 8.09 (d, J=9.29 Hz, 1H), 7.32-7.42 (m, 5H), 7.25-7.32 (m, 1H), 4.67 (s, 4H), 3.95-4.02 (m, 2H).

### 3. Synthesis of 2-((6-nitro-2-(thiazol-5-yl) quinolin-4-yl) oxy) ethan-1-ol

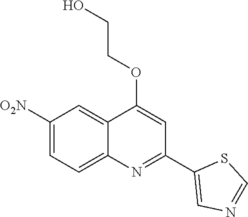 [SMILES: O=[N+]([O-])c1ccc2nc(-c3cncs3)cc(OCCO)c2c1]

To 5-(4-(2-(benzyloxy)ethoxy)-6-nitroquinolin-2-yl)thiazole (0.38 g, 0.13 mmol) was added trifluoroacetic acid (1 mL) at 0° C. The reaction mixture was heated to 80° C. and stirred for 16 h. Then, the volatiles were removed in vacuo to obtain the crude. The crude product 2-((6-nitro-2-(thiazol-5-yl) quinolin-4-yl) oxy) ethan-1-ol (pale brown semi solid, 0.3 g) was taken forward for next step without further purification. LCMS (ESI+): m/z calcd. for C₁₄H₁₂N₃O₄S [M+H]⁺, 318; found, 318.

### 4. Synthesis of 2-((6-amino-2-(thiazol-5-yl) quinolin-4-yl) oxy) ethan-1-ol

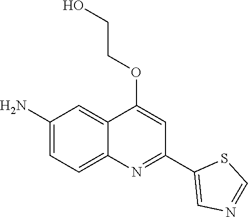 [SMILES: Nc1ccc2nc(-c3cncs3)cc(OCCO)c2c1]

2-((6-Amino-2-(thiazol-5-yl) quinolin-4-yl) oxy) ethan-1-ol was prepared using the procedure for preparation of 4-(2-methoxyethoxy)-2-(thiazol-5-yl) quinolin-6-amine as a brown solid (0.22 g, crude). LCMS (ESI+): m/z calcd. for C₁₄H₁₄N₃O₂S [M+H]⁺, 288; found, 288.

### 5. Synthesis of N-(4-(2-hydroxyethoxy)-2-(thiazol-5-yl) quinolin-6-yl) oxetane-3-carboxamide

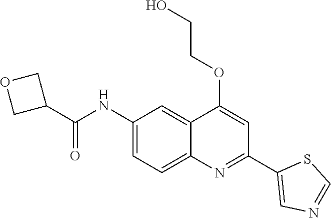 [SMILES: O=C(Nc1ccc2nc(-c3cncs3)cc(OCCO)c2c1)C1COC1]

N-(4-(2-hydroxyethoxy)-2-(thiazol-5-yl) quinolin-6-yl) oxetane-3-carboxamide was prepared using the similar procedure for the preparation of N-(4-(2-methoxyethoxy)-2-(thiazol-5-yl)quinolin-6-yl)oxetane-3-carboxamide as an off-white solid (0.018 g, yield: 12%) after preparative HPLC purification. HPLC purity: 98.61%. LCMS (ESI+): m/z calcd. for C₁₈H₁₈N₃O₄S [M+H]⁺, 372; found, 372. ¹H NMR (400 MHz, DMSO-d₆, δ): 10.31 (s, 1H), 9.18 (s, 1H), 8.82 (s, 1H), 8.48 (s, 1H), 7.83-8.02 (m, 2H), 7.63 (s, 1H), 5.04 (t, J=5.14 Hz, 1H), 4.75-4.72 (m, 4H), 4.41 (br t, J=4.40 Hz, 2H), 4.01 (p, J=7.34 Hz, 1H), 3.92 (q, J=4.40 Hz, 2H).

### Example 10: 2,2,2-trifluoro-N-(4-(2-hydroxyethoxy)-2-(thiazol-5-yl)quinolin-6-yl)acetamide

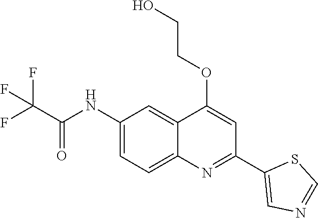 [SMILES: O=C(Nc1ccc2nc(-c3cncs3)cc(OCCO)c2c1)C(F)(F)F]

### 1. Synthesis of 2,2,2-trifluoro-N-(4-(2-hydroxyethoxy)-2-(thiazol-5-yl)quinolin-6-yl)acetamide

To 2-((6-amino-2-(thiazol-5-yl) quinolin-4-yl) oxy) ethan-1-ol (50 mg, 0.13 mmol) was added TFA (1 mL) at 0° C. The reaction was heated to 80° C. and stirred for 16 h. Then, the reaction mixture the volatiles were removed in vacuo and triturated with acetonitrile (5 mL), diethyl ether (5 mL) and dried in vacuo to afford 2,2,2-trifluoro-N-(4-(2-hydroxyethoxy)-2-(thiazol-5-yl)quinolin-6-yl)acetamide as pale brown solid (0.02 g, yield: 40%). HPLC purity: 95.36%. LCMS (ESI+): m/z calcd. for C₁₆H₁₃F₃N₃O₃S [M+H]⁺, 384; found, 384. ¹H NMR (400 MHz, DMSO-d₆, δ): 11.60 (br s, 1H), 9.20 (s, 1H), 8.85 (s, 1H), 8.49 (d, J=1.75 Hz, 1H), 7.97-8.05 (m, 1H), 7.90-7.94 (m, 1H), 7.66 (s, 1H), 5.04 (t, J=5.38 Hz, 1H), 4.43 (t, J=4.75 Hz, 2H), 3.92 (q, J=5.00 Hz, 2H).

### Example 11: N-(4-(2-methoxyethoxy)-2-(2-methylthiazol-5-yl)quinolin-6-yl)oxetane-3-carboxamide

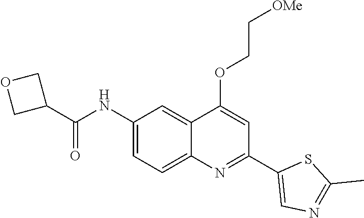 [SMILES: COCCOc1cc(-c2cnc(C)s2)nc2ccc(NC(=O)C3COC3)cc12]

### 1. Synthesis of N-(4-(2-methoxyethoxy)-2-(2-methylthiazol-5-yl)quinolin-6-yl)oxetane-3-carboxamide

N-(4-(2-methoxyethoxy)-2-(2-methylthiazol-5-yl)quinolin-6-yl)oxetane-3-carboxamide was prepared in a similar way as Example 1. HPLC purity: 98.56%. LCMS (ESI+): m/z calcd. for C₂₀H₂₂N₃O₄S [M+H]⁺, 400; found, 400. 1HNMR (400 MHz, DMSO-d₆, δ): 10.29 (s, 1H), 8.53 (s, 1H), 8.43 (s, 1H), 7.96 (d, J=7.2 Hz, 1H), 7.83 (d, J=9.1 Hz, 1H), 7.57 (s, 1H), 4.73 (d, J=7.5 Hz, 4H), 4.50 (d, J=4.0 Hz, 2H), 4.10-3.92 (m, 1H), 3.85 (s, 2H), 3.41 (s, 3H), 2.70 (s, 3H).

### Example 12: N-(2-(2-chlorothiazol-5-yl)-4-ethoxyquinolin-6-yl)oxetane-3-carboxamide

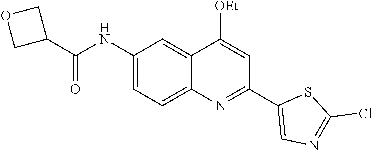 [SMILES: CCOc1cc(-c2cnc(Cl)s2)nc2ccc(NC(=O)C3COC3)cc12]

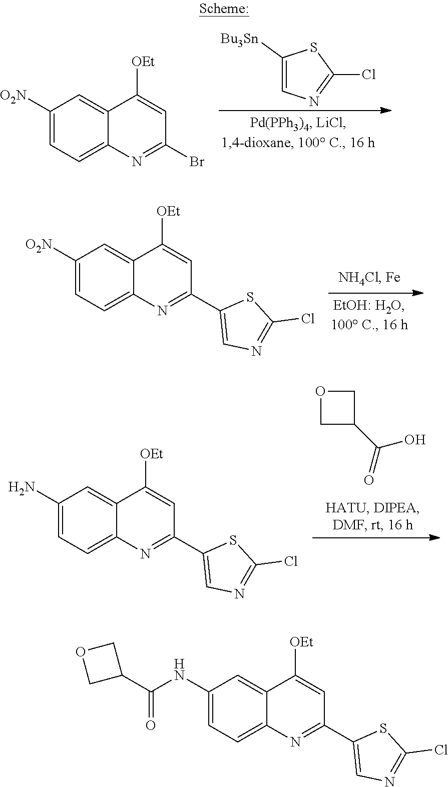

### 1. Synthesis of 2-chloro-5-(4-ethoxy-6-nitroquinolin-2-yl) thiazole

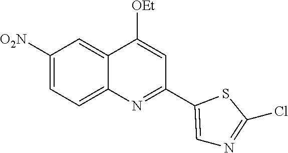 [SMILES: CCOc1cc(-c2cnc(Cl)s2)nc2ccc([N+](=O)[O-])cc12]

2-Chloro-5-(4-ethoxy-6-nitroquinolin-2-yl) thiazole was prepared using the procedure for preparation of 5-(4-(2-methoxyethoxy)-6-nitroquinolin-2-yl) thiazole as an off-white solid (0.21 g, yield: 37%). ¹H NMR (400 MHz, DMSO-d₆, δ): 8.90 (d, J=1.96 Hz, 1H), 8.80 (s, 1H), 8.45 (dd, J=9.29, 2.45 Hz, 1H), 8.07 (d, J=9.29 Hz, 1H), 7.83 (s, 1H), 4.52 (q, J=6.85 Hz, 2H), 0.87 (br t, J=7.09 Hz, 3H).

### 2. Synthesis of 2-(2-chlorothiazol-5-yl)-4-ethoxyquinolin-6-amine

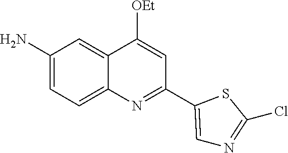 [SMILES: CCOc1cc(-c2cnc(Cl)s2)nc2ccc(N)cc12]

To a stirred solution of 2-chloro-5-(4-ethoxy-6-nitroquinolin-2-yl) thiazole (0.26 g, 0.77 mmol) in EtOH:H₂O (5:1, 12 mL) were added NH₄Cl (0.41 g, 7.73 mmol) and Fe powder (0.34 g, 6.19 mmol) at RT and the reaction mixture was heated to 100° C. and stirred for 5 h. Then, the reaction mixture was filtered through celite and the filtrate was concentrated in vacuo to obtain the 2-(2-chlorothiazol-5-yl)-4-ethoxyquinolin-6-amine as pale brown solid (0.6 g, crude). LCMS (ESI+): m/z calcd. for C₁₄H₁₃ClN₃OS [M+H]⁺, 306; found, 306.

### 3. Synthesis of N-(2-(2-chlorothiazol-5-yl)-4-ethoxyquinolin-6-yl)oxetane-3-carboxamide

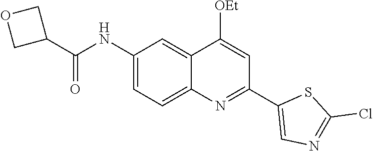 [SMILES: CCOc1cc(-c2cnc(Cl)s2)nc2ccc(NC(=O)C3COC3)cc12]

N-(2-(2-chlorothiazol-5-yl)-4-ethoxyquinolin-6-yl)oxetane-3-carboxamide was prepared using the similar procedure for the preparation of N-(4-(2-methoxyethoxy)-2-(thiazol-5-yl)quinolin-6-yl)oxetane-3-carboxamide as an off-white solid (0.015 g, yield: 20%). HPLC purity: 98.67%. LCMS (ESI+): m/z calcd. for C₁₈H₁₇ClN₃O₃S [M+H]⁺, 390; found, 390. ¹H NMR (400 MHz, DMSO-d₆, δ): 10.30 (s, 1H), 8.61 (s, 1H), 8.51 (d, J=2.25 Hz, 1H), 7.90-7.97 (m, 1H), 7.85 (d, J=9.76 Hz, 1H), 7.60 (s, 1H), 4.76-4.72 (m, 4H), 4.43 (q, J=7.00 Hz, 2H), 4.05-4.01 (m, 1H), 1.53 (t, J=6.94 Hz, 3H).

### Example 13: 2-hydroxy-N-(4-(2-methoxyethoxy)-2-(thiazol-5-yl) quinolin-6-yl) acetamide

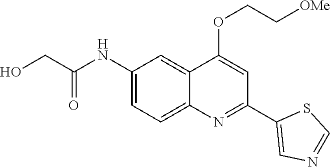 [SMILES: COCCOc1cc(-c2cncs2)nc2ccc(NC(=O)CO)cc12]

### 1. Synthesis of 2-hydroxy-N-(4-(2-methoxyethoxy)-2-(thiazol-5-yl) quinolin-6-yl) acetamide

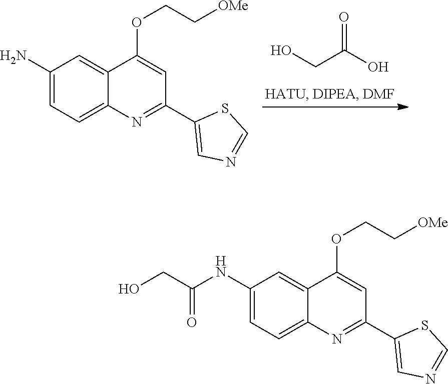 [SMILES: COCCOc1cc(-c2cncs2)nc2ccc(N)cc12 ; O=C(O)CO ; COCCOc1cc(-c2cncs2)nc2ccc(NC(=O)CO)cc12]

To a stirred solution of 2-hydroxyacetic acid (0.05 g, 0.72 mmol, 1 eq) was added HATU (0.41 g, 1.17 mmol, 1.5 eq) and stirred for 10 min at RT. To this was added 4-(2-methoxyethoxy)-2-(thiazol-5-yl)quinolin-6-amine (0.196 g, 0.65 mmol, 1eq), followed by addition of DIPEA (0.25 mL, 1.84 mmol, 2 eq) at RT. The reaction was stirred at RT for 16 h. Then, the reaction mixture was diluted with water (50 mL) and extracted with 10% MeOH in DCM (2×50 mL). The organic layer was washed with brine (50 mL), dried over anhydrous Na₂SO₄, filtered and concentrated in vacuo. The crude obtained was purified by preparative HPLC purification to afford the title compound as an off-white solid (0.023 g, yield: 9%). HPLC purity: 98.74%. LCMS (ESI+): m/z calcd. for C₁₇H₁₈N₃O₄S [M+H]⁺, 360; found, 360. ¹H NMR (400 MHz, DMSO-d₆, δ): 10.07 (s, 1H), 9.18 (s, 1H), 8.81 (s, 1H), 8.58 (d, J=2.25 Hz, 1H), 8.04 (dd, J=9.13, 2.38 Hz, 1H), 7.86 (d, J=9.13 Hz, 1H), 7.63 (s, 1H), 5.65 (t, J=6.00 Hz, 1H), 4.53 (dd, J=5.25, 3.63 Hz, 2H), 4.06 (d, J=6.13 Hz, 2H), 3.84-3.90 (m, 2H), 3.42 (s, 3H).

### Example 14: (S)-2-hydroxy-N-(4-(2-methoxyethoxy)-2-(thiazol-5-yl)quinolin-6-yl)propanamide

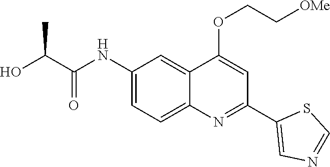 [SMILES: COCCOc1cc(-c2cncs2)nc2ccc(NC(=O)[C@H](C)O)cc12]

### 1. Synthesis of(S)-2-hydroxy-N-(4-(2-methoxyethoxy)-2-(thiazol-5-yl)quinolin-6-yl)propanamide

Using a similar procedure as Example 13, (S)-2-hydroxy-N-(4-(2-methoxyethoxy)-2-(thiazol-5-yl)quinolin-6-yl)propanamide was prepared as white solid (0.047 g, yield: 16%). HPLC purity: 97.46%. LCMS (ESI+): m/z calcd. for C₁₈H₂₀N₃O₄S [M+H]⁺, 374; found, 374. ¹H NMR (400 MHz, DMSO-d₆, δ): 10.03 (s, 1H), 9.18 (s, 1H), 8.80 (s, 1H), 8.60 (d, J=2.38 Hz, 1H), 8.03 (dd, J=9.07, 2.44 Hz, 1H), 7.85 (d, J=9.13 Hz, 1H), 7.63 (s, 1H), 5.73 (d, J=5.25 Hz, 1H), 4.52 (dd, J=5.25, 3.63 Hz, 2H), 4.15-4.25 (m, 1H), 3.78-3.92 (m, 2H), 3.41 (s, 3H), 1.35 (d, J=6.75 Hz, 3H).

### Example 15: (R)-2-hydroxy-N-(4-(2-methoxyethoxy)-2-(thiazol-5-yl)quinolin-6-yl)propanamide

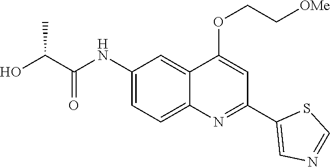 [SMILES: COCCOc1cc(-c2cncs2)nc2ccc(NC(=O)[C@@H](C)O)cc12]

### 1. Synthesis of (R)-2-hydroxy-N-(4-(2-methoxyethoxy)-2-(thiazol-5-yl)quinolin-6-yl)propanamide

Using the similar procedure as Example 13, (R)-2-hydroxy-N-(4-(2-methoxyethoxy)-2-(thiazol-5-yl)quinolin-6-yl)propanamide was prepared as white solid (0.03 g, yield: 24%). HPLC purity: 97.62%. LCMS (ESI+): m/z calcd. for C₁₈H₂₀N₃O₄S [M+H]⁺, 374; found, 374. ¹H NMR (400 MHz, DMSO-d₆, δ): 10.06 (s, 1H), 9.18 (s, 1H), 8.80 (s, 1H), 8.60 (d, J=1.47 Hz, 1H), 8.03 (dd, J=9.29, 1.96 Hz, 1H), 7.85 (d, J=8.80 Hz, 1H), 7.63 (s, 1H), 5.76 (br s, 1H), 4.52 (t, J=4.40 Hz, 3H), 4.21 (br s, 1H), 3.87 (br t, J=3.91 Hz, 2H), 3.41 (s, 2H), 1.34 (d, J=6.36 Hz, 3H).

### Example 16: cis-3-hydroxy-N-(4-(2-methoxyethoxy)-2-(thiazol-5-yl)quinolin-6-yl)cyclobutane-1-carboxamide

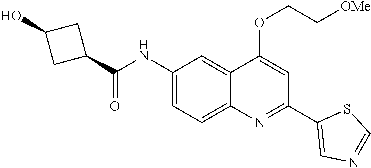 [SMILES: COCCOc1cc(-c2cncs2)nc2ccc(NC(=O)[C@H]3C[C@@H](O)C3)cc12]

### 1. Synthesis of cis-3-hydroxy-N-(4-(2-methoxyethoxy)-2-(thiazol-5-yl)quinolin-6-yl)cyclobutane-1-carboxamide

Using the similar procedure as Example 13, cis-3-hydroxy-N-(4-(2-methoxyethoxy)-2-(thiazol-5-yl)quinolin-6-yl)cyclobutane-1-carboxamide was prepared as off-white solid (0.03 g, yield: 23%). HPLC purity: 97.23%. LCMS (ESI+): m/z calcd. for C₂₀H₂₂N₃O₄S [M+H]⁺, 400; found, 400. ¹H NMR (400 MHz, DMSO-d₆, δ): 10.17 (s, 1H), 9.17 (s, 1H), 8.79 (s, 1H), 8.44 (d, J=2.13 Hz, 1H), 7.95 (dd, J=9.07, 2.31 Hz, 1H), 7.84 (d, J=9.13 Hz, 1H), 7.62 (s, 1H), 5.16 (d, J=7.00 Hz, 1H), 4.42-4.58 (m, 2H), 3.96-4.07 (m, 1H), 3.86 (dd, J=5.13, 3.63 Hz, 2H), 3.41 (s, 3H), 2.59-2.73 (m, 1H), 2.31-2.44 (m, 2H), 2.01-2.14 (m, 2H).

### Example 17: trans-3-hydroxy-N-(4-(2-methoxyethoxy)-2-(thiazol-5-yl)quinolin-6-yl)cyclobutane-1-carboxamide

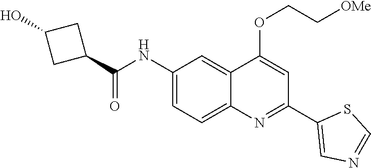 [SMILES: COCCOc1cc(-c2cncs2)nc2ccc(NC(=O)[C@H]3C[C@H](O)C3)cc12]

### 1. Synthesis of trans-3-hydroxy-N-(4-(2-methoxyethoxy)-2-(thiazol-5-yl)quinolin-6-yl)cyclobutane-1-carboxamide

Using the similar procedure as Example 13, trans-3-hydroxy-N-(4-(2-methoxyethoxy)-2-(thiazol-5-yl)quinolin-6-yl)cyclobutane-1-carboxamide was prepared as off-white solid (0.03 g, yield: 25%). HPLC purity: 99.20%. LCMS (ESI+): m/z calcd. for C₂₀H₂₂N₃O₄S [M+H]⁺, 400; found, 400. ¹H NMR (400 MHz, DMSO-d₆, δ): 10.17 (s, 1H), 9.17 (s, 1H), 8.79 (s, 1H), 8.45 (d, J=2.25 Hz, 1H), 7.94-7.92 (m, 1H), 7.85-7.82 (m, 1H), 7.62 (s, 1H), 5.10 (d, J=6.4 Hz, 1H), 4.54-4.51 (m, 2H), 4.39-4.29 (m, 1H), 3.88-3.85 (m, 2H), 3.41 (s, 3H), 3.13-3.10 (m, 1H), 2.47-2.41 (m, 2H), 2.14-2.06 (m, 2H).

### Example 18: N-(4-(2-methoxyethoxy)-2-(thiazol-5-yl)quinolin-6-yl)-2-methylcyclopropane-1-carboxamide

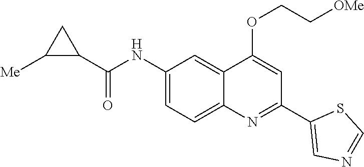 [SMILES: COCCOc1cc(-c2cncs2)nc2ccc(NC(=O)C3CC3C)cc12]

### 1. Synthesis of N-(4-(2-methoxyethoxy)-2-(thiazol-5-yl)quinolin-6-yl)-2-methylcyclopropane-1-carboxamide

Using the similar procedure as Example 13, N-(4-(2-methoxyethoxy)-2-(thiazol-5-yl)quinolin-6-yl)-2-methylcyclopropane-1-carboxamide was prepared as off-white solid (0.03 g, yield: 24%). HPLC purity: 99.20%. LCMS (ESI+): m/z calcd. for C₂₀H₂₂N₃O₃S [M+H]⁺, 384; found, 384. ¹H NMR (400 MHz, DMSO-d₆, δ): 10.49 (s, 1H), 9.17 (s, 1H), 8.79 (s, 1H), 8.42 (d, J=2.13 Hz, 1H), 7.94 (dd, J=9.13, 2.38 Hz, 1H), 7.79-7.88 (m, 1H), 7.61 (s, 1H), 4.48-4.55 (m, 2H), 3.83-3.97 (m, 2H), 3.40 (s, 3H), 1.56-1.60 (m, 1H), 1.21-1.36 (m, 1H), 1.12 (d, J=6.00 Hz, 3H), 1.04-1.09 (m, 1H), 0.64-0.74 (m, 1H).

### Example 19: trans-2-cyano-N-(4-(2-methoxyethoxy)-2-(thiazol-5-yl)quinolin-6-yl)cyclopropane-1-carboxamide

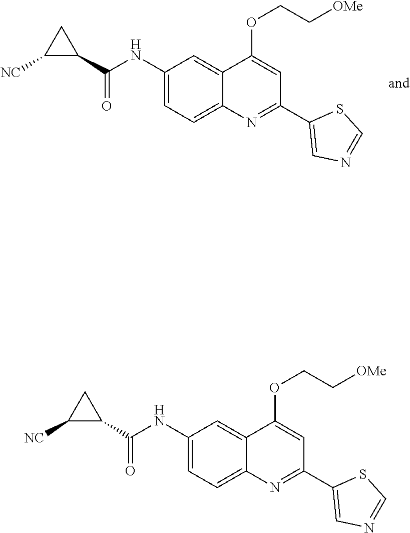 [SMILES: COCCOc1cc(-c2cncs2)nc2ccc(NC(=O)[C@@H]3C[C@H]3C#N)cc12 ; COCCOc1cc(-c2cncs2)nc2ccc(NC(=O)[C@H]3C[C@@H]3C#N)cc12]

### 1. Synthesis of trans-2-cyano-N-(4-(2-methoxyethoxy)-2-(thiazol-5-yl)quinolin-6-yl)cyclopropane-1-carboxamide

Using the similar procedure as Example 13, and trans-2-cyanocyclopropane-1-carboxylic acid as the starting material, trans-2-cyano-N-(4-(2-methoxyethoxy)-2-(thiazol-5-yl)quinolin-6-yl)cyclopropane-1-carboxamide was prepared as off-white solid (0.095 g, yield: 50%). HPLC purity: 99.20%. LCMS (ESI+): m/z calcd. for C₂₀H₁₉N₄O₃S [M+H]⁺, 395; found, 395. ¹H NMR (400 MHz, DMSO-d₆, δ): 10.88 (s, 1H), 9.18 (s, 1H), 8.80 (s, 1H), 8.42 (d, J=2.13 Hz, 1H), 7.93 (t, J=2.25 Hz, 1H), 7.86-7.90 (m, 1H), 7.64 (s, 1H), 4.54-4.50 (m, 2H), 3.78-3.91 (m, 2H), 3.39 (s, 3H), 2.53-2.56 (m, 1H), 2.15-2.18 (m, 1H), 1.64-1.50 (m, 1H), 1.38-1.51 (m, 1H).

### Example 20: N-(4-(2-methoxyethoxy)-2-(thiazol-5-yl)quinolin-6-yl)-2-(trifluoromethyl)cyclopropane-1-carboxamide

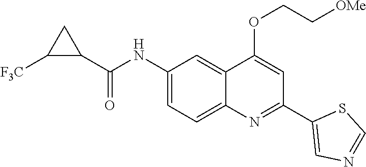 [SMILES: COCCOc1cc(-c2cncs2)nc2ccc(NC(=O)C3CC3C(F)(F)F)cc12]

### 1. Synthesis of N-(4-(2-methoxyethoxy)-2-(thiazol-5-yl)quinolin-6-yl)-2-(trifluoromethyl)cyclopropane-1-carboxamide

Using the similar procedure as Example 13, N-(4-(2-methoxyethoxy)-2-(thiazol-5-yl)quinolin-6-yl)-2-(trifluoromethyl)cyclopropane-1-carboxamide was prepared as off-white solid (0.04 g, yield: 27%). HPLC purity: 97.49%. LCMS (ESI+): m/z calcd. for C₂₀H₁₉F₃N₃O₃S [M+H]⁺, 438; found, 438. ¹H NMR (400 MHz, DMSO-d₆, δ): 10.83 (s, 1H), 9.19 (s, 1H), 8.81 (s, 1H), 8.44 (d, J=2.13 Hz, 1H), 7.91-7.96 (m, 1H), 7.86-7.91 (m, 1H), 7.65 (s, 1H), 4.50-4.56 (m, 2H), 3.84-3.90 (m, 2H), 3.40 (s, 3H), 2.31-2.39 (m, 2H), 1.29-1.42 (m, 2H).

### Example 21: 2-methoxy-N-(4-(2-methoxyethoxy)-2-(thiazol-5-yl)quinolin-6-yl)acetamide

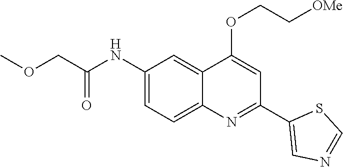 [SMILES: COCCOc1cc(-c2cncs2)nc2ccc(NC(=O)COC)cc12]

### 1. Synthesis of 2-methoxy-N-(4-(2-methoxyethoxy)-2-(thiazol-5-yl)quinolin-6-yl)acetamide

Using the similar procedure as Example 13, 2-methoxy-N-(4-(2-methoxyethoxy)-2-(thiazol-5-yl)quinolin-6-yl)acetamide was prepared as pale brown solid (0.03 g, yield: 25%). HPLC purity: 99.78%. LCMS (ESI+): m/z calcd. for C₁₈H₂₀N₃O₄S [M+H]⁺, 374; found, 374. ¹H NMR (400 MHz, DMSO-d₆, δ): 10.17 (s, 1H), 9.18 (s, 1H), 8.81 (s, 1H), 8.52 (d, J=2.45 Hz, 1H), 8.02 (dd, J=9.05, 2.20 Hz, 1H), 7.86 (d, J=9.29 Hz, 1H), 7.63 (s, 1H), 4.47-4.56 (m, 2H), 4.06 (s, 2H), 3.84-3.88 (m, 2H), 3.41 (s, 3H), 3.40 (s, 3H).

### Example 22: 2-methoxy-N-(4-(2-methoxyethoxy)-2-(thiazol-5-yl)quinolin-6-yl)propanamide

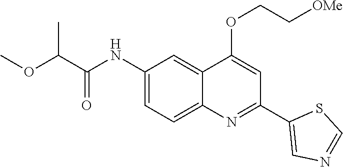 [SMILES: COCCOc1cc(-c2cncs2)nc2ccc(NC(=O)C(C)OC)cc12]

### 1. Synthesis of 2-methoxy-N-(4-(2-methoxyethoxy)-2-(thiazol-5-yl)quinolin-6-yl)propanamide

Using the similar procedure as Example 13, 2-methoxy-N-(4-(2-methoxyethoxy)-2-(thiazol-5-yl)quinolin-6-yl)propanamide was prepared as off-white solid (0.028 g, yield: 22%). HPLC purity: 98.44%. LCMS (ESI+): m/z calcd. for C₁₉H₂₂N₃O₄S [M+H]⁺, 388; found, 388. ¹H NMR (400 MHz, DMSO-d₆, δ): 10.20 (s, 1H), 9.18 (s, 1H), 8.80 (s, 1H), 8.55 (d, J=2.25 Hz, 1H), 8.05 (dd, J=9.13, 2.38 Hz, 1H), 7.86 (d, J=9.01 Hz, 1H), 7.63 (s, 1H), 4.49-4.55 (m, 2H), 3.93 (q, J=6.71 Hz, 1H), 3.84-3.88 (m, 2H), 3.41 (s, 3H), 3.34 (s, 3H), 1.36 (d, J=6.75 Hz, 3H).

### Example 23: N-(4-(2-methoxyethoxy)-2-(thiazol-5-yl)quinolin-6-yl)tetrahydro-2H-pyran-3-carboxamide

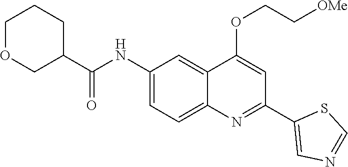 [SMILES: COCCOc1cc(-c2cncs2)nc2ccc(NC(=O)C3CCCOC3)cc12]

### 1. Synthesis of N-(4-(2-methoxyethoxy)-2-(thiazol-5-yl)quinolin-6-yl)tetrahydro-2H-pyran-3-carboxamide

Using the similar procedure as Example 13, N-(4-(2-methoxyethoxy)-2-(thiazol-5-yl)quinolin-6-yl)tetrahydro-2H-pyran-3-carboxamide was prepared as off-white solid (0.036 g, yield: 26%). HPLC purity: 99.54%. LCMS (ESI+): m/z calcd. for C₂₁H₂₄N₃O₄S [M+H]+, 414; found, 414. ¹H NMR (400 MHz, DMSO-d₆, δ): 10.31 (s, 1H), 9.18 (s, 1H), 8.80 (s, 1H), 8.45 (d, J=2.25 Hz, 1H), 7.90-7.96 (m, 1H), 7.83-7.88 (m, 1H), 7.63 (s, 1H), 4.52 (dd, J=5.38, 3.63 Hz, 2H), 3.98-4.05 (m, 1H), 3.75-3.92 (m, 3H), 3.42-3.44 (m, 1H), 3.41 (s, 3H), 3.32-3.38 (m, 1H), 2.65-2.74 (m, 1H), 1.95-2.04 (m, 1H), 1.70-1.82 (m, 1H), 1.50-1.70 (m, 2H).

### Example 24: 3-methoxy-N-(4-(2-methoxyethoxy)-2-(thiazol-5-yl)quinolin-6-yl)propanamide

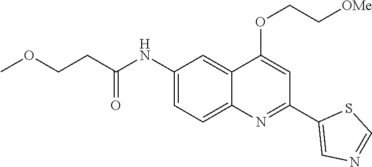 [SMILES: COCCOc1cc(-c2cncs2)nc2ccc(NC(=O)CCOC)cc12]

### 1. Synthesis of 3-methoxy-N-(4-(2-methoxyethoxy)-2-(thiazol-5-yl)quinolin-6-yl)propanamide

Using the similiar procedure as Example 13, 3-methoxy-N-(4-(2-methoxyethoxy)-2-(thiazol-5-yl)quinolin-6-yl)propanamide was prepared as off-white solid (0.04 g, yield: 31%). HPLC purity: 97.11%. LCMS (ESI+): m/z calcd. for C₁₉H₂₂N₃O₄S [M+H]+, 388; found, 388. ¹H NMR (400 MHz, DMSO-d₆, δ): 10.31 (s, 1H), 9.17 (s, 1H), 8.79 (s, 1H), 8.44 (d, J=1.96 Hz, 1H), 7.94 (dd, J=9.29, 2.45 Hz, 1H), 7.85 (d, J=8.31 Hz, 1H), 7.62 (s, 1H), 4.49-4.54 (m, 2H), 3.84-3.88 (m, 2H), 3.66 (t, J=6.11 Hz, 2H), 3.41 (s, 3H), 3.26 (s, 3H), 2.61 (br t, J=6.11 Hz, 2H).

### Example 25: N-(4-(2-methoxyethoxy)-2-(thiazol-5-yl)quinolin-6-yl)-2-propoxypropanamide

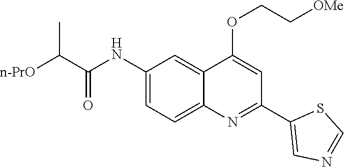

### 1. Synthesis of N-(4-(2-methoxyethoxy)-2-(thiazol-5-yl)quinolin-6-yl)-2-propoxypropanamide

Using the similiar procedure as Example 13, N-(4-(2-methoxyethoxy)-2-(thiazol-5-yl)quinolin-6-yl)-2-propoxypropanamide was prepared as off-white solid (0.025 g, yield: 12%). HPLC purity: 97.46%. LCMS (ESI+): m/z calcd. for C₂₁H₂₆N₃O₄S [M+H]+, 416; found, 416. ¹H NMR (400 MHz, DMSO-d₆, δ): 9.18 (s, 1H) 10.13 (s, 1H), 8.80 (s, 1H), 8.53 (d, J=2.13 Hz, 1H), 8.02 (dd, J=9.13, 2.25 Hz, 1H), 7.86 (d, J=9.13 Hz, 1H), 7.63 (s, 1H), 4.53 (t, J=4.25 Hz, 2H), 4.01 (q, J=6.59 Hz, 1H), 3.86 (t, J=4.25 Hz, 2H), 3.43-3.51 (m, 1H), 3.41 (s, 3H), 3.34-3.40 (m, 1H), 1.52-1.66 (m, 2H), 1.36 (d, J=6.63 Hz, 3H), 0.90 (t, J=7.38 Hz, 3H).

### Example 26: 2-ethoxy-N-(4-(2-methoxyethoxy)-2-(thiazol-5-yl)quinolin-6-yl)propanamide

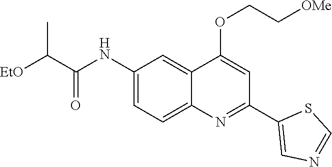 [SMILES: CCOC(C)C(=O)Nc1ccc2nc(-c3cncs3)cc(OCCOC)c2c1]

### 1. Synthesis of 2-ethoxy-N-(4-(2-methoxyethoxy)-2-(thiazol-5-yl)quinolin-6-yl)propanamide

Using the similiar procedure as Example 13, 2-ethoxy-N-(4-(2-methoxyethoxy)-2-(thiazol-5-yl)quinolin-6-yl)propanamide was prepared as pale brown solid (0.03 g, yield: 22%). HPLC purity: 97.46%. LCMS (ESI+): m/z calcd. for C₂₀H₂₄N₃O₄S [M+H]+, 402; found, 402. ¹H NMR (400 MHz, DMSO-d₆, δ): 10.17 (s, 1H), 9.18 (s, 1H), 8.81 (s, 1H), 8.54 (d, J=1.96 Hz, 1H), 8.03 (dd, J=9.05, 1.96 Hz, 1H), 7.86 (d, J=9.05 Hz, 1H), 7.64 (s, 1H), 4.52 (t, J=3.91 Hz 2H), 4.02 (q, J=6.60 Hz, 1H), 3.86 (t, J=3.91 Hz 2H), 3.43-3.62 (m, 2H), 3.40 (s, 3H), 1.35 (d, J=6.60 Hz, 3H), 1.19 (t, J=6.97 Hz, 3H).

### Example 27: 2,2-difluoro-3-hydroxy-N-(4-(2-methoxyethoxy)-2-(thiazol-5-yl)quinolin-6-yl)propanamide

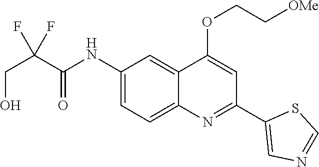 [SMILES: COCCOc1cc(-c2cncs2)nc2ccc(NC(=O)C(F)(F)CO)cc12]

### 1. Synthesis of 2,2-difluoro-3-hydroxy-N-(4-(2-methoxyethoxy)-2-(thiazol-5-yl)quinolin-6-yl)propanamide

Using the similiar procedure as Example 13, 2,2-difluoro-3-hydroxy-N-(4-(2-methoxyethoxy)-2-(thiazol-5-yl)quinolin-6-yl)propanamide was prepared as off-white solid (0.025 g, yield: 15%). HPLC purity: 99.20%. LCMS (ESI+): m/z calcd. for C₁₈H₁₈F₂N₃O₄S [M+H]+, 410; found, 410. ¹H NMR (400 MHz, DMSO-d₆, δ): 10.88 (br s, 1H), 9.20 (s, 1H), 8.83 (s, 1H), 8.59 (d, J=1.47 Hz, 1H), 8.08 (dd, J=9.05, 1.71 Hz, 1H), 7.91 (d, J=9.29 Hz, 1H), 7.67 (s, 1H), 5.82 (br s, 1H), 4.39-4.65 (m, 2H), 3.94 (br t, J=13.45 Hz, 2H), 3.84-3.88 (m, 2H), 3.41 (s, 3H).

### Example 28: 2-cyano-N-(4-(2-methoxyethoxy)-2-(thiazol-5-yl)quinolin-6-yl)propanamide

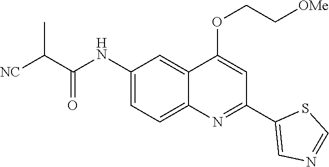 [SMILES: COCCOc1cc(-c2cncs2)nc2ccc(NC(=O)C(C)C#N)cc12]

### 1. Synthesis of 2-cyano-N-(4-(2-methoxyethoxy)-2-(thiazol-5-yl)quinolin-6-yl)propanamide

Using the similiar procedure as Example 13, 2-cyano-N-(4-(2-methoxyethoxy)-2-(thiazol-5-yl)quinolin-6-yl)propanamide was prepared as pale yellow solid (0.16 g, yield: 44%). HPLC purity: 99.51%. LCMS (ESI+): m/z calcd. for C₁₉H₁₉N₄O₃S [M+H]+, 383; found, 383. ¹H NMR (400 MHz, DMSO-d₆, δ): 10.73 (s, 1H), 9.19 (s, 1H), 8.82 (s, 1H), 8.41 (d, J=1.2 Hz, 1H), 7.96-7.88 (m, 2H), 7.66 (s, 1H), 4.53 (t, J=3.6 Hz, 2H), 4.01 (q, J=7.2 Hz, 1H), 3.87 (t, J=4.4 Hz, 2H), 3.41 (s, 3H), 1.56 (d, J=7.6 Hz, 3H).

### Example 29: 1-methoxy-N-(4-(2-methoxyethoxy)-2-(thiazol-5-yl)quinolin-6-yl)cyclopropane-1-carboxamide

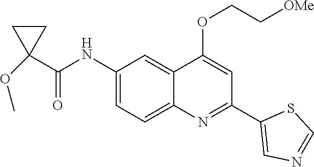 [SMILES: COCCOc1cc(-c2cncs2)nc2ccc(NC(=O)C3(OC)CC3)cc12]

### 1. Synthesis of 1-methoxy-N-(4-(2-methoxyethoxy)-2-(thiazol-5-yl)quinolin-6-yl)cyclopropane-1-carboxamide

Using the similiar procedure as Example 13, 1-methoxy-N-(4-(2-methoxyethoxy)-2-(thiazol-5-yl)quinolin-6-yl)cyclopropane-1-carboxamide was prepared as off-white solid (0.035 g, yield: 26%). HPLC purity: 99.56%. LCMS (ESI+): m/z calcd. for C₂₀H₂₂N₃O₄S [M+H]+, 400; found, 400. ¹H NMR (400 MHz, DMSO-d₆, δ): 10.31 (s, 1H), 9.18 (s, 1H), 8.81 (s, 1H), 8.61 (d, J=1.47 Hz, 1H), 8.09 (dd, J=9.05, 1.71 Hz, 1H), 7.85 (d, J=9.29 Hz, 1H), 7.63 (s, 1H), 4.52 (t, J=4.40 Hz, 2H), 3.86 (t, J=3.42 Hz, 2H), 3.40 (s, 3H), 3.37 (s, 3H), 1.25-1.20 (m, 2H), 1.12-1.19 (m, 2H).

### Example 30: 3,3-difluoro-N-(4-(2-methoxyethoxy)-2-(thiazol-5-yl)quinolin-6-yl)cyclobutane-1-carboxamide

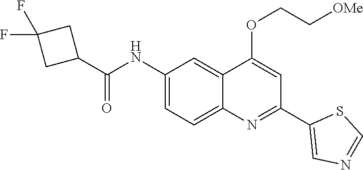 [SMILES: COCCOc1cc(-c2cncs2)nc2ccc(NC(=O)C3CC(F)(F)C3)cc12]

### 1. Synthesis of 3,3-difluoro-N-(4-(2-methoxyethoxy)-2-(thiazol-5-yl)quinolin-6-yl)cyclobutane-1-carboxamide

Using the similiar procedure as Example 13, 3,3-difluoro-N-(4-(2-methoxyethoxy)-2-(thiazol-5-yl)quinolin-6-yl)cyclobutane-1-carboxamide was prepared as white solid (0.03 g, yield: 21%). HPLC purity: 99.81%. LCMS (ESI+): m/z calcd. for C₂₀H₂₀F₂N₃O₃S [M+H]+, 420; found, 420. ¹H NMR (400 MHz, DMSO-d₆, δ): 10.44 (s, 1H), 9.18 (s, 1H), 8.80 (s, 1H), 8.44 (d, J=2.25 Hz, 1H), 7.92-7.99 (m, 1H), 7.86-7.89 (m, 1H), 7.63 (s, 1H), 4.53 (dd, J=5.32, 3.69 Hz, 2H), 3.84-3.90 (m, 2H), 3.41 (s, 3H), 3.12-3.20 (m, 1H), 2.75-2.92 (m, 4H).

### Example 31: N-(4-(2-methoxyethoxy)-2-(thiazol-5-yl)quinolin-6-yl)bicyclo[1.1.1]pentane-1-carboxamide

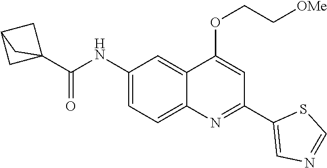 [SMILES: COCCOc1cc(-c2cncs2)nc2ccc(NC(=O)C34CC(C3)C4)cc12]

### 1. Synthesis of N-(4-(2-methoxyethoxy)-2-(thiazol-5-yl)quinolin-6-yl)bicyclo[1.1.1]pentane-1-carboxamide

Using the similiar procedure as Example 13, N-(4-(2-methoxyethoxy)-2-(thiazol-5-yl)quinolin-6-yl)bicyclo[1.1.1]pentane-1-carboxamide was prepared as off-white solid (0.03 g, yield: 23%). HPLC purity: 99.82%. LCMS (ESI+): m/z calcd. for C₂₁H₂₂N₃O₃S [M+H]+, 396; found, 396. ¹H NMR (400 MHz, DMSO-d₆, δ): 9.88 (s, 1H), 9.17 (s, 1H), 8.80 (s, 1H), 8.42 (d, J=2.25 Hz, 1H), 8.07 (dd, J=9.13, 2.38 Hz, 1H), 7.85 (d, J=9.13 Hz, 1H), 7.62 (s, 1H), 4.49-4.56 (m, 2H), 3.82-3.92 (m, 2H), 3.40 (s, 3H), 2.07-2.15 (m, 7H).

### Example 32: 3-methoxy-N-(4-(2-methoxyethoxy)-2-(thiazol-5-yl)quinolin-6-yl)-2-methylpropanamide

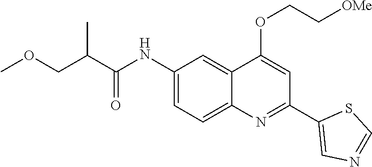 [SMILES: COCCOc1cc(-c2cncs2)nc2ccc(NC(=O)C(C)COC)cc12]

### 1. Synthesis of 3-methoxy-N-(4-(2-methoxyethoxy)-2-(thiazol-5-yl)quinolin-6-yl)-2-methylpropanamide

Using the similiar procedure as Example 13, 3-methoxy-N-(4-(2-methoxyethoxy)-2-(thiazol-5-yl)quinolin-6-yl)-2-methylpropanamide was prepared as off-white solid (0.032 g, yield: 24%). HPLC purity: 98.94%. LCMS (ESI+): m/z calcd. for C₂₀H₂₄N₃O₄S [M+H]+, 402; found, 402. ¹H NMR (400 MHz, CDCl₃, δ) 8.86 (s, 1H), 8.50 (s, 1H), 8.42 (d, J=2.38 Hz, 1H), 8.39 (s, 1H), 7.95 (d, J=9.01 Hz, 1H), 7.79 (dd, J=9.01, 2.38 Hz, 1H), 7.13 (s, 1H), 4.44 (t, J=4.63 Hz, 2H), 3.93-3.97 (m, 2H), 3.60 (d, J=6.25 Hz, 2H), 3.53 (s, 3H), 3.48 (s, 3H), 2.69-2.80 (m, 1H), 1.27 (d, J=7.25 Hz, 3H).

### Example 33: cis-3-methoxy-N-(4-(2-methoxyethoxy)-2-(thiazol-5-yl)quinolin-6-yl)cyclobutane-1-carboxamide

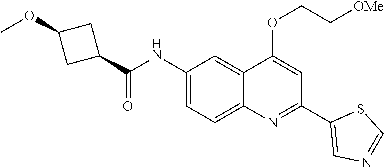 [SMILES: COCCOc1cc(-c2cncs2)nc2ccc(NC(=O)[C@H]3C[C@@H](OC)C3)cc12]

### 1. Synthesis of cis-3-methoxy-N-(4-(2-methoxyethoxy)-2-(thiazol-5-yl)quinolin-6-yl)cyclobutane-1-carboxamide

Using the similiar procedure as Example 13, cis-3-methoxy-N-(4-(2-methoxyethoxy)-2-(thiazol-5-yl)quinolin-6-yl)cyclobutane-1-carboxamide was prepared as off-white solid (0.04 g, yield: 29%). HPLC purity: 96.09%. LCMS (ESI+): m/z calcd. for C₂₁H₂₄N₃O₄S [M+H]⁺, 414; found, 414. ¹H NMR (400 MHz, DMSO-d₆, δ): 10.20 (s, 1H), 9.15 (s, 1H), 8.77 (s, 1H), 8.42 (s, 1H), 7.93-7.93 (m, 1H), 7.85 (d, J=8.80 Hz, 1H), 7.60 (s, 1H), 4.46-4.56 (m, 2H), 3.77-3.86 (m, 3H), 3.39 (s, 3H), 3.13 (s, 3H), 2.72-2.79 (m, 1H), 2.39-2.49 (m, 2H), 2.08-2.03 (m, 2H).

### Example 34: trans-3-methoxy-N-(4-(2-methoxyethoxy)-2-(thiazol-5-yl)quinolin-6-yl)cyclobutane-1-carboxamide

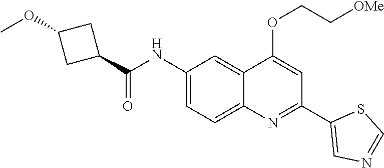 [SMILES: COCCOc1cc(-c2cncs2)nc2ccc(NC(=O)[C@H]3C[C@H](OC)C3)cc12]

### 1. Synthesis of trans-3-methoxy-N-(4-(2-methoxyethoxy)-2-(thiazol-5-yl)quinolin-6-yl)cyclobutane-1-carboxamide

Using the similiar procedure as Example 13, trans-3-methoxy-N-(4-(2-methoxyethoxy)-2-(thiazol-5-yl)quinolin-6-yl)cyclobutane-1-carboxamide was prepared as pale yellow solid (0.04 g, yield: 29%). HPLC purity: 98.63%. LCMS (ESI+): m/z calcd. for C₂₁H₂₄N₃O₄S [M+H]⁺, 414; found, 414. ¹H NMR (400 MHz, DMSO-d₆, δ): 10.23 (s, 1H), 9.17 (s, 1H), 8.80 (s, 1H), 8.46 (s, 1H), 7.93-7.98 (m, 1H), 7.85 (d, J=8.80 Hz, 1H), 7.62 (s, 1H), 4.46-4.56 (m, 2H), 3.99-4.10 (m, 1H), 3.82-3.90 (m, 2H), 3.41 (s, 3H), 3.16 (s, 3H), 3.18-3.23 (m, 1H), 2.39-2.48 (m, 2H), 2.09-2.22 (m, 2H).

### Example 35: N-(4-(2-methoxyethoxy)-2-(thiazol-5-yl)quinolin-6-yl)isoxazole-4-carboxamide

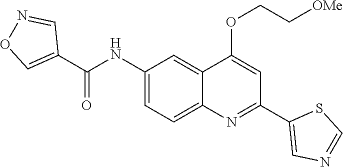 [SMILES: COCCOc1cc(-c2cncs2)nc2ccc(NC(=O)c3cnoc3)cc12]

### 1. Synthesis of N-(4-(2-methoxyethoxy)-2-(thiazol-5-yl)quinolin-6-yl)isoxazole-4-carboxamide

Using the similiar procedure as Example 13, N-(4-(2-methoxyethoxy)-2-(thiazol-5-yl)quinolin-6-yl)isoxazole-4-carboxamide was prepared as off-white solid (0.01 g, yield: 8%). HPLC purity: 97.10%. LCMS (ESI+): m/z calcd. for C₁₉H₁₇N₄O₄S [M+H]⁺, 397; found, 397. ¹H NMR (400 MHz, DMSO-d₆, δ): 10.59 (s, 1H), 9.64 (s, 1H), 9.19 (s, 1H), 9.14 (s, 1H), 8.82 (s, 1H), 8.47 (d, J=2.38 Hz, 1H), 8.12 (dd, J=9.19, 2.44 Hz, 1H), 7.93 (d, J=9.01 Hz, 1H), 7.66 (s, 1H), 4.53-4.57 (m, 2H), 3.84-3.93 (m, 2H), 3.41 (s, 3H).

### Example 36: (S)-2-methoxy-N-(4-(2-methoxyethoxy)-2-(thiazol-5-yl)quinolin-6-yl)propanamide

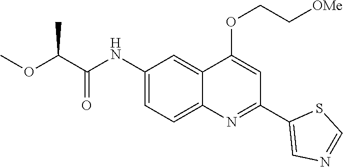 [SMILES: COCCOc1cc(-c2cncs2)nc2ccc(NC(=O)[C@H](C)OC)cc12]

### 1. Synthesis of(S)-2-methoxy-N-(4-(2-methoxyethoxy)-2-(thiazol-5-yl)quinolin-6-yl)propanamide

Using the similiar procedure as Example 13, (S)-2-methoxy-N-(4-(2-methoxyethoxy)-2-(thiazol-5-yl)quinolin-6-yl)propanamide was prepared as yellow solid (0.07 g, yield: 57%). HPLC purity: 99.82%. LCMS (ESI+): m/z calcd. for C₁₉H₂₂N₃O₄S [M+H]⁺, 388; found, 388. ¹H NMR (400 MHz, DMSO-d₆, δ): 10.20 (s, 1H), 9.18 (s, 1H), 8.80 (s, 1H), 8.55 (d, J=1.96 Hz, 1H), 8.05 (dd, J=9.29, 2.45 Hz, 1H), 7.86 (d, J=8.80 Hz, 1H), 7.63 (s, 1H), 4.50-4.56 (m, 2H), 3.93 (q, J=6.68 Hz, 1H), 3.85-3.89 (m, 2H), 3.41 (s, 3H), 3.34 (s, 3H), 1.36 (d, J=6.85 Hz, 3H).

### Example 37: (R)-2-methoxy-N-(4-(2-methoxyethoxy)-2-(thiazol-5-yl)quinolin-6-yl)propanamide

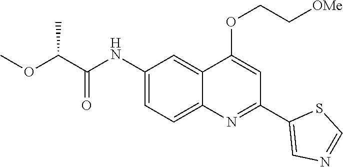 [SMILES: COCCOc1cc(-c2cncs2)nc2ccc(NC(=O)[C@@H](C)OC)cc12]

### 1. Synthesis of (R)-2-methoxy-N-(4-(2-methoxyethoxy)-2-(thiazol-5-yl)quinolin-6-yl)propanamide

Using the similiar procedure as Example 13, (R)-2-methoxy-N-(4-(2-methoxyethoxy)-2-(thiazol-5-yl)quinolin-6-yl)propanamide was prepared as pale yellow solid (0.07 g, yield: 55%). HPLC purity: 99.82%. LCMS (ESI+): m/z calcd. for C₁₉H₂₂N₃O₄S [M+H]⁺, 388; found, 388. ¹H NMR (400 MHz, DMSO-d₆, δ): 10.20 (s, 1H), 9.18 (s, 1H), 8.80 (s, 1H), 8.55 (d, J=2.45 Hz, 1H), 8.05 (dd, J=9.05, 2.20 Hz, 1H), 7.86 (d, J=8.80 Hz, 1H), 7.63 (s, 1H), 4.50-4.56 (m, 2H), 3.93 (q, J=6.68 Hz, 1H), 3.85-3.89 (m, 2H), 3.41 (s, 3H), 3.34 (s, 3H), 1.36 (d, J=6.85 Hz, 3H).

### Example 38: N-(4-((1-methoxypropan-2-yl)oxy)-2-(thiazol-5-yl)quinolin-6-yl)oxetane-3-carboxamide

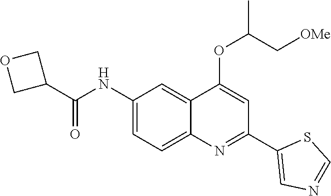 [SMILES: COCC(C)Oc1cc(-c2cncs2)nc2ccc(NC(=O)C3COC3)cc12]

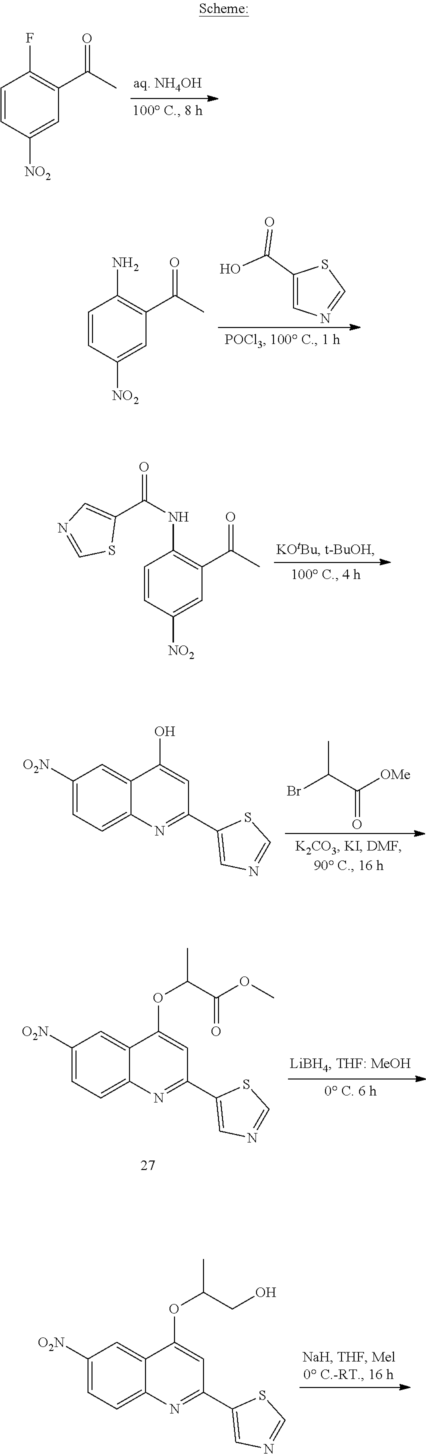

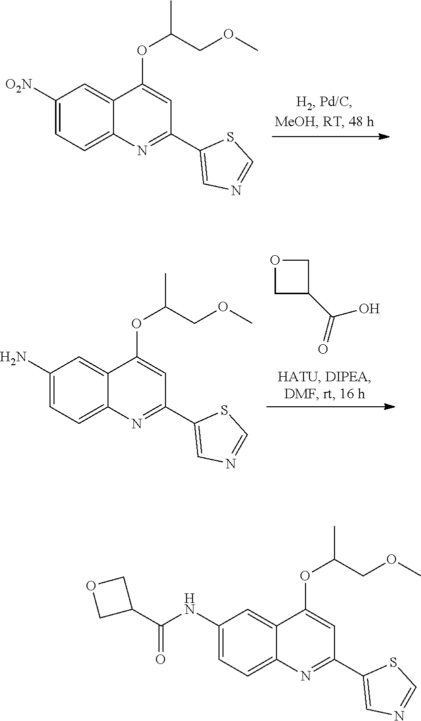

### 1. Synthesis of 1-(2-amino-5-nitrophenyl)ethan-1-one

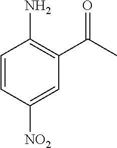

A mixture of 1-(2-fluoro-5-nitrophenyl)ethan-1-one (28 g, 152.88 mmol) and NH₄OH (25%, 230 mL) was heated in an autoclave at 100° C. and stirred for 8 h. Then, the reaction mixture was filtered, and washed with ice-cold water (100 mL) and dried under vacuum. The crude was triturated with acetonitrile (100 mL) and dried in vacuo to 1-(2-amino-5-nitrophenyl)ethan-1-one (17 g, yield: 75%) as yellow solid. ¹H NMR (400 MHz, DMSO-d₆, δ): 8.63 (d, J=2.45 Hz, 1H), 8.45 (brs, 2H), 8.09 (dd, J=9.29, 2.45 Hz, 1H), 6.88 (d, J=9.29 Hz, 1H), 2.62 (s, 3H).

### 2. Synthesis of N-(2-acetyl-4-nitrophenyl)thiazole-5-carboxamide

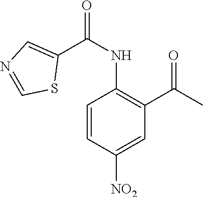 [SMILES: CC(=O)c1cc([N+](=O)[O-])ccc1NC(=O)c1cncs1]

A mixture of 1-(2-amino-5-nitrophenyl)ethan-1-one (19.53 g, 108.40 mmol) and thiazole-5-carboxylic acid (14 g, 108.40 mmol) was added POCl₃ (200 mL) was heated to 100° C. for 1 h. Then, the reaction mixture was cooled to RT and quenched with ice-cold water (200 mL). The solid obtained was filtered and dried under vacuum. The crude was triturated with diethyl ether (200 mL) and dried under vacuum to afford N-(2-acetyl-4-nitrophenyl)thiazole-5-carboxamide (22 g, yield: 70%) as yellow solid. LCMS (ESI+): m/z calcd. for C₁₂H₁₀N₃O₄S [M+H]⁺, 292; found, 292. ¹H NMR (400 MHz, DMSO-d₆, δ): 12.39 (s, 1H), 9.43 (s, 1H), 8.79 (br d, J=1.96 Hz, 1H), 8.48-8.65 (m, 3H), 2.78 (s, 3H).

### 3. Synthesis of 6-nitro-2-(thiazol-5-yl)quinolin-4-ol

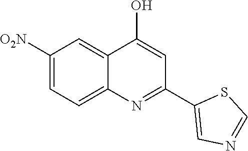 [SMILES: O=[N+]([O-])c1ccc2nc(-c3cncs3)cc(O)c2c1]

To a stirred solution of N-(2-acetyl-4-nitrophenyl)thiazole-5-carboxamide (11 g, 37.80 mmol) in t-butanol (110 mL) was added t-BuOK (8.48 g, 75.60 mmol) at RT and the reaction mixture was heated to 100° C. for 4 h. Then, the reaction mixture was filtered and the solid obtained was mixed with 1 N HCl (200 mL) and stirred for 10 min. The obtained solid was filtered and dried under vacuum. The crude was triturated with acetonitrile (50 mL) and dried under in vacuo to afford 6-nitro-2-(thiazol-5-yl)quinolin-4-ol as yellow solid (7.5 g, yield:73%). LCMS (ESI+): m/z calcd. for C₁₂H₈N₃O₃S [M+H]⁺, 274; found, 274. ¹H NMR (400 MHz, DMSO-d₆, δ): 12.74 (br s, 1H), 9.35 (s, 1H), 8.86 (br s, 1H), 8.69 (s, 1H), 8.39-8.54 (m, 1H), 7.97 (br d, J=9.29 Hz, 1H), 6.67 (br s, 1H).

### 4. Synthesis of methyl 2-((6-nitro-2-(thiazol-5-yl)quinolin-4-yl)oxy)propanoate

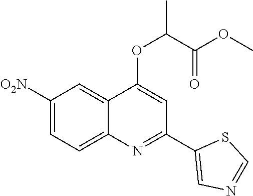 [SMILES: COC(=O)C(C)Oc1cc(-c2cncs2)nc2ccc([N+](=O)[O-])cc12]

To a stirred solution of 6-nitro-2-(thiazol-5-yl)quinolin-4-ol (2.5 g, 9.15 mmol) in DMF (15 mL) were added K₂CO₃ (3.15 g, 22.89 mmol), KI (0.15 g, 0.91 mmol) and methyl 2-bromopropanoate (2.29 g, 13.71 mmol) at RT and the reaction mixture was heated to 90° C. and stirred for 16 h. The reaction mixture was stirred at RT for 16 h. Then, the reaction mixture was poured into ice cold water (100 mL), the solid obtained was filtered, washed with water (100 mL) and dried under vacuo. The crude was triturated with diethyl ether (25 mL) to afford methyl 2-((6-nitro-2-(thiazol-5-yl)quinolin-4-yl)oxy)propanoate as pale brown solid (2.2 g, yield: 67%). LCMS (ESI+): m/z calcd. for C₁₆H₁₄N₃O₅S [M+H]⁺, 360; found, 360. ¹H NMR (400 MHz, DMSO-d₆, δ): 9.30 (s, 1H), 8.94 (br d, J=4.89 Hz, 2H), 8.47 (dd, J=9.29, 1.96 Hz, 1H), 8.11 (br d, J=9.29 Hz, 1H), 7.85 (s, 1H), 5.78 (q, J=6.85 Hz, 1H), 3.75 (s, 3H), 1.77 (br d, J=6.85 Hz, 3H).

### 5. Synthesis of 2-((6-nitro-2-(thiazol-5-yl)quinolin-4-yl)oxy)propan-1-ol

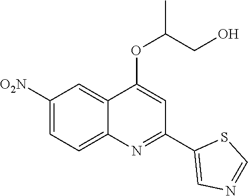 [SMILES: CC(CO)Oc1cc(-c2cncs2)nc2ccc([N+](=O)[O-])cc12]

To a stirred solution of methyl 2-((6-nitro-2-(thiazol-5-yl)quinolin-4-yl)oxy)propanoate (1.1 g, 30.06 mmol) in MeOH:THF (1:1, 24 mL) was added LiBH₄ (2.0 M sol. in THF, 9.1 mL, 18.36 mmol) at 0° C. dropwise for 20 min. The reaction mixture was stirred at the same temperature for 6 h. Then, the reaction mixture was quenched with saturated NH₄Cl (50 mL). The aqueous layer was extracted with EtOAc (2×75 mL). The combined organic layer was washed with brine (50 mL), dried over anhydrous Na₂SO₄, filtered and concentrated in vacuo to afford 2-((6-nitro-2-(thiazol-5-yl)quinolin-4-yl)oxy)propan-1-ol (0.38 g, yield:30%) as pale brown solid. LCMS (ESI+): m/z calcd. for C₁₅H₁₄N₃O₄S [M+H]⁺, 332; found, 332. ¹H NMR (400 MHz, DMSO-d₆, δ): 9.29 (s, 1H), 8.97-9.08 (m, 2H), 8.44 (dd, J=9.29, 2.45 Hz, 1H), 8.08 (d, J=9.29 Hz, 1H), 7.88 (s, 1H), 5.09-5.22 (m, 2H), 3.75 (br t, J=5.14 Hz, 2H), 1.41 (d, J=5.87 Hz, 3H).

### 6. Synthesis of 5-(4-((1-methoxypropan-2-yl)oxy)-6-nitroquinolin-2-yl)thiazole

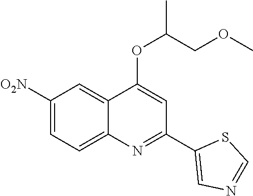 [SMILES: COCC(C)Oc1cc(-c2cncs2)nc2ccc([N+](=O)[O-])cc12]

To a stirred solution of 2-((6-nitro-2-(thiazol-5-yl)quinolin-4-yl)oxy)propan-1-ol (0.38 g, 1.14 mmol) in THF (18 mL) was added NaH (60% suspension in mineral oil, 0.03 g, 1.25 mmol) at 0° C., to this was added methyl iodide (0.08 mL, 1.25 mmol) at 0° C. and the reaction mixture was stirred at the RT for 16 h. Then, the reaction mixture was quenched with saturated NH₄Cl (50 mL). The aqueous layer was extracted with EtOAc (2×50 mL). The combined organic layer was washed with brine (50 mL), dried over anhydrous Na₂SO₄, filtered and concentrated in vacuo to afford 5-(4-((1-methoxypropan-2-yl)oxy)-6-nitroquinolin-2-yl)thiazole (0.25 g, yield: 63%) as brown solid. LCMS (ESI+): m/z calcd. for C₁₆H₁₆N₃O₄S [M+H]⁺, 346; found, 346. ¹H NMR (400 MHz, DMSO-d₆, δ): 9.29 (d, J=1.96 Hz, 1H), 8.99 (d, J=3.91 Hz, 1H), 8.89 (s, 1H), 8.40-8.49 (m, 1H), 8.09 (dd, J=9.29, 5.87 Hz, 1H), 7.89-7.86 (m, 1H), 5.27-5.42 (m, 0.5H), 4.35-4.56 (m, 1H), 3.87-3.98 (m, 0.5H), 3.66-3.79 (m, 1H), 3.42-3.36 (m, 3H), 1.30-1.46 (m, 3H).

### 7. Synthesis of 4-((1-methoxypropan-2-yl)oxy)-2-(thiazol-5-yl)quinolin-6-amine

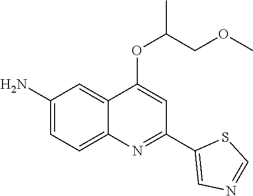 [SMILES: COCC(C)Oc1cc(-c2cncs2)nc2ccc(N)cc12]

4-((1-Methoxypropan-2-yl)oxy)-2-(thiazol-5-yl)quinolin-6-amine was prepared using the procedure followed for preparation of 4-(2-methoxyethoxy)-2-(thiazol-5-yl) quinolin-6-amine. The crude was purified by trituration with diethyl ether (25 mL) and n-pentane (25 mL) to give title product as a brown solid (0.18 g, yield: 78%). LCMS (ESI+): m/z calcd. for C₁₆H₁₈N₃O₂S [M+H]⁺, 316; found, 316. ¹H NMR (400 MHz, DMSO-d₆, δ): 9.08 (s, 1H), 8.67 (br s, 1H), 7.60 (br dd, J=8.80, 4.89 Hz, 1H), 7.45 (br d, J=16.63 Hz, 1H), 7.11 (br d, J=8.31 Hz, 1H), 7.05 (br d, J=11.25 Hz, 1H), 5.67 (br d, J=12.72 Hz, 1.6H), 5.16-5.14 (m, 0.4H), 4.27 (br d, J=3.91 Hz, 1H), 3.85-3.82 (m, 1H), 3.62-3.66 (m, 1H), 3.41-3.35 (m, 3H), 1.22-1.51 (m, 3H).

### 8. Synthesis of N-(4-((1-methoxypropan-2-yl)oxy)-2-(thiazol-5-yl)quinolin-6-yl)oxetane-3-carboxamide

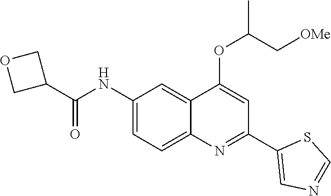 [SMILES: COCC(C)Oc1cc(-c2cncs2)nc2ccc(NC(=O)C3COC3)cc12]

Using the same procedure as N-(4-(2-methoxyethoxy)-2-(thiazol-5-yl)quinolin-6-yl)oxetane-3-carboxamide, N-(4-((1-methoxypropan-2-yl)oxy)-2-(thiazol-5-yl)quinolin-6-yl)oxetane-3-carboxamide was prepared as off-white solid (0.18 g, yield: 81%). HPLC purity: 99.16%. LCMS (ESI+): m/z calcd. for C₂₀H₂₂N₃O₄S [M+H]⁺, 400; found, 400. ¹H NMR (400 MHz, DMSO-d₆, δ): 10.28 (d, J=3.50 Hz, 1H), 9.17 (d, J=1.25 Hz, 1H), 8.82 (d, J=2.75 Hz, 1H), 8.51 (d, J=2.25 Hz, 0.5H), 8.42 (d, J=2.25 Hz, 0.5H), 7.91-7.99 (m, 1H), 7.86 (dd, J=8.75, 5.13 Hz, 1H), 7.65 (d, J=16.01 Hz, 1H), 5.17-5.30 (m, 1H), 4.76-4.734 (m, 4H), 4.29-4.41 (m, 1H), 3.97-4.07 (m, 0.5H), 3.82-3.92 (m, 0.5H), 3.63-3.74 (m, 1H), 3.43 (s, 1.5H), 3.36 (s, 1.5H), 1.39 (d, 6.25 Hz, 1.5H), 1.33 (d, 6.25 Hz, 1.5H).

### Example 39: N-(4-((1-methoxy-2-methylpropan-2-yl)oxy)-2-(thiazol-5-yl)quinolin-6-yl)oxetane-3-carboxamide

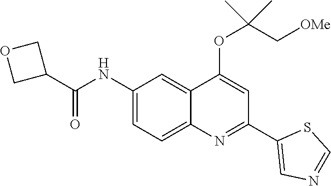 [SMILES: COCC(C)(C)Oc1cc(-c2cncs2)nc2ccc(NC(=O)C3COC3)cc12]

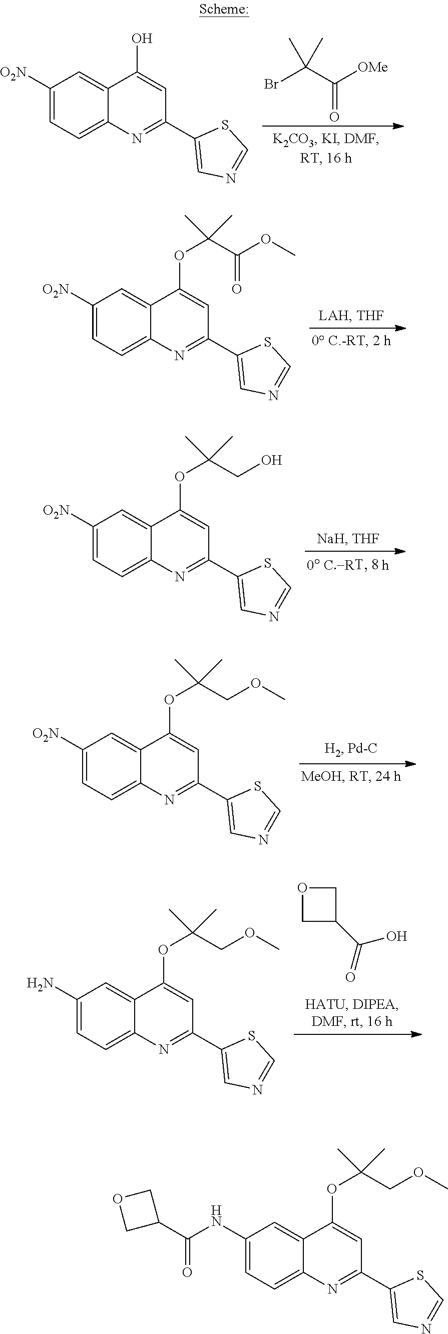

### 1. Synthesis methyl 2-methyl-2-((6-nitro-2-(thiazol-5-yl)quinolin-4-yl)oxy)propanoate

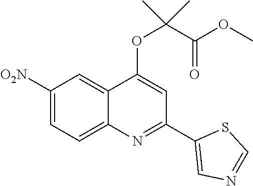 [SMILES: COC(=O)C(C)(C)Oc1cc(-c2cncs2)nc2ccc([N+](=O)[O-])cc12]

Methyl 2-methyl-2-((6-nitro-2-(thiazol-5-yl)quinolin-4-yl)oxy)propanoate was prepared using the procedure followed for preparation of methyl 2-((6-nitro-2-(thiazol-5-yl)quinolin-4-yl)oxy)propanoate. The obtained compound was purified by combi flash chromatography (1% MeOH in CH₂Cl₂) to give title product as a pale-yellow solid (0.8 g, yield: 23%). LCMS (ESI+): m/z calcd. for C₁₇H₁₆N₃O₅S [M+H]⁺, 374; found, 374. ¹H NMR (400 MHz, DMSO-d₆, δ): 9.30 (s, 1H), 8.95 (d, J=2.45 Hz, 1H), 8.76 (s, 1H), 8.48 (dd, J=9.05, 2.69 Hz, 1H), 8.12 (d, J=9.29 Hz, 1H), 7.21 (s, 1H), 3.77 (s, 3H), 1.88 (s, 6H).

### 2. Synthesis of 2-methyl-2-((6-nitro-2-(thiazol-5-yl)quinolin-4-yl)oxy)propan-1-ol

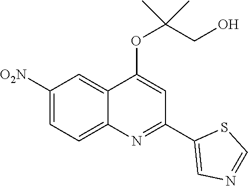 [SMILES: CC(C)(CO)Oc1cc(-c2cncs2)nc2ccc([N+](=O)[O-])cc12]

To a stirred solution of Methyl 2-methyl-2-((6-nitro-2-(thiazol-5-yl)quinolin-4-yl)oxy)propanoate (0.5 g, 1.33 mmol) in THF (1, 15 mL) was added LAH (1.0 M sol. in THF, 2.0 mL, 2.00 mmol) dropwise at 0° C. for 15 min and the reaction mixture was warmed to RT and for 2 h. Then, the reaction mixture was quenched with saturated NH₄Cl (50 mL). The aqueous layer was extracted with EtOAc (2×50 mL). The combined organic layer was washed with brine (50 mL), dried over anhydrous Na₂SO₄, filtered and concentrated in vacuo to obtain the crude. The crude was purified by combiflash chromatography (80% EtOAc in heptane) to afford 2-methyl-2-((6-nitro-2-(thiazol-5-yl)quinolin-4-yl)oxy)propan-1-ol (0.14 g, yield: 30%) as pale brown solid. LCMS (ESI+): m/z calcd. for C₁₆H₁₆N₃O₄S [M+H]⁺, 346; found, 346. ¹H NMR (400 MHz, DMSO-d₆, δ): 9.29 (s, 1H), 9.09 (d, J=2.45 Hz, 1H), 9.01 (s, 1H), 8.45 (dd, J=9.05, 2.20 Hz, 1H), 8.09 (d, J=9.29 Hz, 1H), 7.85 (s, 1H), 5.07 (s, 1H), 4.23 (s, 2H), 1.36 (s, 6H).

### 3. Synthesis of 5-(4-((1-methoxy-2-methylpropan-2-yl)oxy)-6-nitroquinolin-2-yl)thiazole

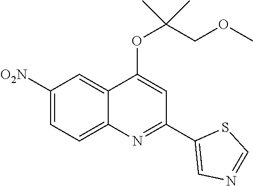 [SMILES: COCC(C)(C)Oc1cc(-c2cncs2)nc2ccc([N+](=O)[O-])cc12]

5-(4-((1-Methoxy-2-methylpropan-2-yl)oxy)-6-nitroquinolin-2-yl)thiazole was prepared using the procedure followed for preparation of 5-(4-((1-methoxypropan-2-yl)oxy)-6-nitroquinolin-2-yl)thiazole. The obtained compound was purified by combi flash chromatography (1% MeOH in CH₂Cl₂) to give the title product as brown solid (0.17 g, yield: 54%). LCMS (ESI+): m/z calcd. for C₁₇H₁₈N₃O₄S [M+H]⁺, 360; found, 360. ¹H NMR (400 MHz, DMSO-d₆, δ): 9.30 (s, 1H), 9.00 (s, 1H), 8.90 (d, J=2.45 Hz, 1H), 8.46 (dd, J=9.05, 2.69 Hz, 1H), 8.10 (d, J=9.29 Hz, 1H), 7.90 (s, 1H), 4.37 (s, 2H), 3.26 (s, 3H), 1.37 (s, 6H).

### 4. Synthesis of 4-((1-methoxy-2-methylpropan-2-yl)oxy)-2-(thiazol-5-yl)quinolin-6-amine

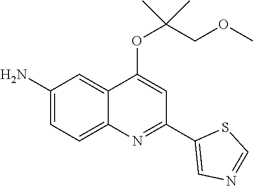 [SMILES: COCC(C)(C)Oc1cc(-c2cncs2)nc2ccc(N)cc12]

4-((1-Methoxy-2-methylpropan-2-yl)oxy)-2-(thiazol-5-yl)quinolin-6-amine was prepared using the procedure followed for preparation of 4-(2-methoxyethoxy)-2-(thiazol-5-yl) quinolin-6-amine. The crude compound was triturated with diethyl ether to obtain the title compound as brown solid (0.15 g, yield: 95%). LCMS (ESI+): m/z calcd. for C₁₇H₂₀N₃O₂S [M+H]⁺, 330; found, 330. ¹H NMR (400 MHz, DMSO-d₆, δ): 9.09 (br s, 1H), 8.45-8.30 (br s, 1H), 8.36 (br s, 2H), 7.60 (br d, J=8.80 Hz, 1H), 7.39-7.54 (m, 1H), 7.06-7.15 (m, 2H), 4.17 (s, 2H), 3.24 (s, 3H), 1.35 (s, 6H).

### 5. Synthesis of N-(4-((1-methoxy-2-methylpropan-2-yl)oxy)-2-(thiazol-5-yl)quinolin-6-yl)oxetane-3-carboxamide

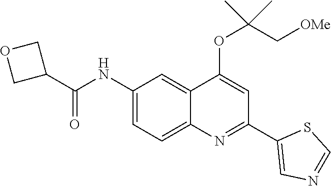 [SMILES: COCC(C)(C)Oc1cc(-c2cncs2)nc2ccc(NC(=O)C3COC3)cc12]

Using the same procedure as N-(4-(2-methoxyethoxy)-2-(thiazol-5-yl)quinolin-6-yl)oxetane-3-carboxamide, N-(4-((1-methoxy-2-methylpropan-2-yl)oxy)-2-(thiazol-5-yl)quinolin-6-yl)oxetane-3-carboxamide was prepared as off-white solid (0.18 g, yield: 82%). LCMS (ESI+): m/z calcd. for C₂₁H₂₄N₃O₄S [M+H]⁺, 414; found, 414. HPLC purity: 99.70%. ¹H NMR (400 MHz, DMSO-d₆, δ): 10.29 (s, 1H), 9.17 (s, 1H), 8.82 (s, 1H), 8.51 (d, J=1.96 Hz, 1H), 7.99 (dd, J=9.29, 1.96 Hz, 1H), 7.87 (d, J=9.29 Hz, 1H), 7.65 (s, 1H), 4.76-4.72 (m, 4H), 4.26 (s, 2H), 3.07-4.07 (m, 1H), 3.26 (s, 3H), 2.50 (s, 6H).

### Example 40: N-(2-(4-cyclopropyl-1H-imidazol-1-yl)-4-(2-methoxyethoxy)quinolin-6-yl)oxetane-3-carboxamide

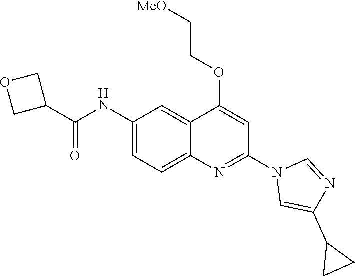 [SMILES: COCCOc1cc(-n2cnc(C3CC3)c2)nc2ccc(NC(=O)C3COC3)cc12]

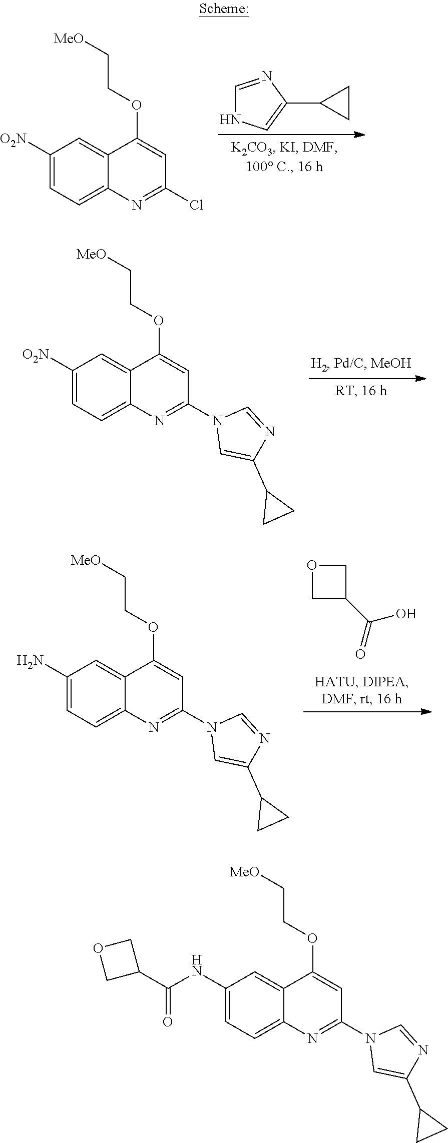

### 1. Synthesis of 2-(4-cyclopropyl-1H-imidazol-1-yl)-4-(2-methoxyethoxy)-6-nitroquinoline

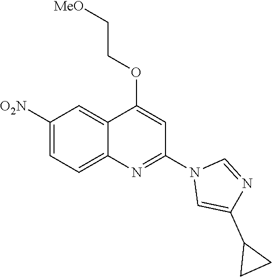 [SMILES: COCCOc1cc(-n2cnc(C3CC3)c2)nc2ccc([N+](=O)[O-])cc12]

2-(4-Cyclopropyl-1H-imidazol-1-yl)-4-(2-methoxyethoxy)-6-nitroquinoline was prepared using the procedure followed for preparation of 4-ethoxy-2-(4-methyl-1H-imidazol-1-yl)-6-nitroquinoline. The title product was obtained as brown solid (0.22 g, yield: 58%). LCMS (ESI+): m/z calcd. for C₁₈H₁₉N₄O₄ [M+H]⁺, 355; found, 355. ¹H NMR (400 MHz, DMSO-d₆, δ): 8.87 (br s, 1H), 8.65 (s, 1H), 8.47 (br d, J=9.39 Hz, 1H), 8.03 (br d, J=9.00 Hz, 1H), 7.93 (s, 1H), 7.55 (s, 1H), 4.60 (br s, 2H), 3.90 (br s, 2H), 3.40 (s, 3H), 1.84-1.99 (m, 1H), 0.83-0.91 (m, 2H), 0.76-0.74 (m, 2H).

### 2. Synthesis of 2-(4-cyclopropyl-1H-imidazol-1-yl)-4-(2-methoxyethoxy)quinolin-6-amine

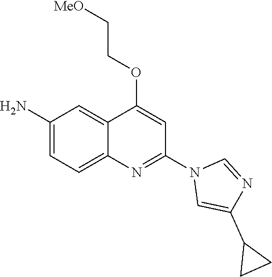 [SMILES: COCCOc1cc(-n2cnc(C3CC3)c2)nc2ccc(N)cc12]

2-(4-Cyclopropyl-1H-imidazol-1-yl)-4-(2-methoxyethoxy)quinolin-6-amine was prepared using the procedure followed for preparation of 4-ethoxy-2-(4-methyl-1H-imidazol-1-yl) quinolin-6-amine as brown solid (5 g, yield: 78%). LCMS (ESI+): m/z calcd. for C₁₈H₂₁N₄O₂ [M+H]⁺, 325; found, 325. ¹H NMR (400 MHz, DMSO-d₆, δ): 8.42 (s, 1H), 7.76 (s, 1H), 7.57 (d, J=8.80 Hz, 1H), 7.16 (s, 1H), 7.12 (br dd, J=9.05, 2.20 Hz, 1H), 7.06 (br d, J=1.96 Hz, 1H), 5.61 (s, 2H), 4.44 (t, J=4.89 Hz, 2H), 3.83 (br t, J=4.40 Hz, 2H), 3.38 (s, 3H), 1.83-1.92 (m, 1H), 0.78-0.87 (m, 2H), 0.69-0.73 (m, 2H).

### 3. Synthesis of N-(2-(4-cyclopropyl-1H-imidazol-1-yl)-4-(2-methoxyethoxy)quinolin-6-yl)oxetane-3-carboxamide

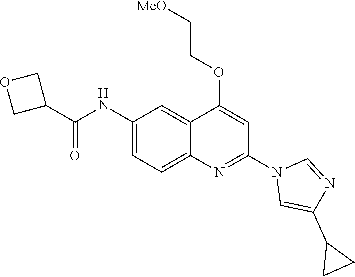 [SMILES: COCCOc1cc(-n2cnc(C3CC3)c2)nc2ccc(NC(=O)C3COC3)cc12]

Using the same procedure as N-(4-(2-methoxyethoxy)-2-(thiazol-5-yl)quinolin-6-yl)oxetane-3-carboxamide, N-(2-(4-cyclopropyl-1H-imidazol-1-yl)-4-(2-methoxyethoxy)quinolin-6-yl)oxetane-3-carboxamide was prepared as white solid (0.05 g, yield: 24%). HPLC purity: 99.82%. LCMS (ESI+): m/z calcd. for C₂₂H₂₅N₄O₄ [M+H]⁺, 409; found, 409. ¹H NMR (400 MHz, DMSO-d₆, δ): 10.28 (s, 1H), 8.53 (d, J=1.25 Hz, 1H), 8.44 (d, J=2.25 Hz, 1H), 7.98 (dd, J=9.01, 2.38 Hz, 1H), 7.79-7.88 (m, 2H), 7.34 (s, 1H), 4.75-4.71 (m, 4H), 4.49-4.56 (m, 2H), 3.96-4.07 (m, 1H), 3.87-3.84 (m, 2H), 3.41 (s, 3H), 1.85-1.95 (m, 1H), 0.80-0.88 (m, 2H), 0.69-0.77 (m, 2H).

### Example 41: N-(4-(2-methoxyethoxy)-2-(4-methyl-1H-imidazol-1-yl)quinolin-6-yl)oxetane-3-carboxamide

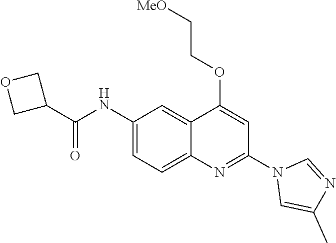 [SMILES: COCCOc1cc(-n2cnc(C)c2)nc2ccc(NC(=O)C3COC3)cc12]

N-(4-(2-methoxyethoxy)-2-(4-methyl-1H-imidazol-1-yl)quinolin-6-yl)oxetane-3-carboxamide was prepared following the procedure of Example 40 as off white solid (0.06 g, yield: 26%). HPLC purity: 97.16%. LCMS (ESI+): m/z calcd. for C₂₀H₂₃N₄O₄ [M+H]⁺, 383; found, 383. ¹H NMR (400 MHz, DMSO-d₆, δ): 10.29 (s, 1H), 8.59 (s, 1H), 8.45 (d, J=2.38 Hz, 1H), 7.99 (dd, J=9.07, 2.44 Hz, 1H), 7.80-7.87 (m, 2H), 7.37 (s, 1H), 4.74 (d, J=7.50 Hz, 4H), 4.50-4.58 (m, 2H), 3.98-4.05 (m, 1H), 3.86 (dd, J=5.13, 3.63 Hz, 2H), 3.41 (s, 3H), 2.21 (s, 3H).

### Example 42: N-(4-(2-hydroxyethoxy)-2-(1H-imidazol-1-yl)quinolin-6-yl)oxetane-3-carboxamide

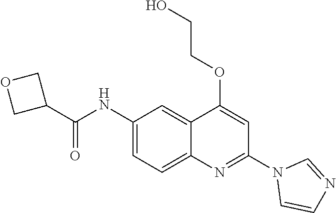 [SMILES: O=C(Nc1ccc2nc(-n3ccnc3)cc(OCCO)c2c1)C1COC1]

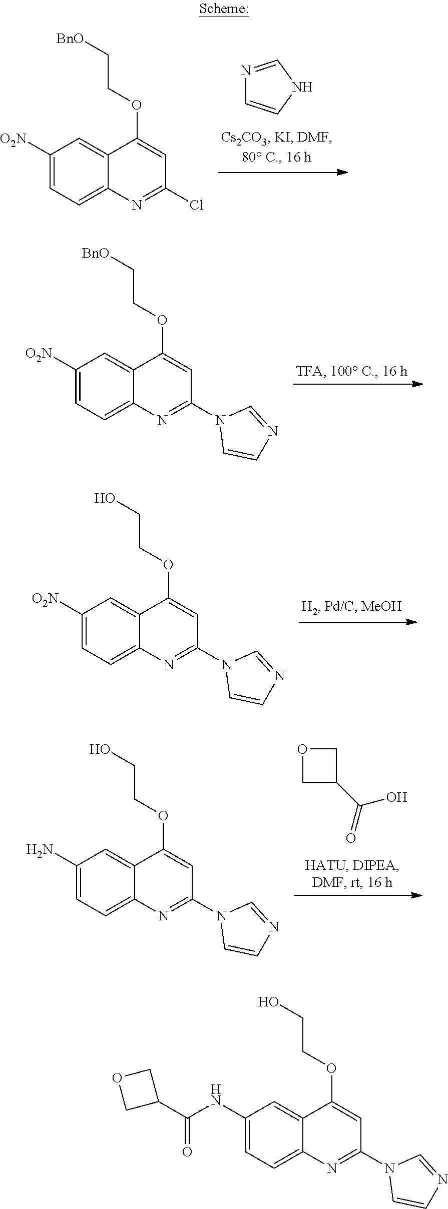

### 1. Synthesis of 4-(2-(benzyloxy)ethoxy)-2-(1H-imidazol-1-yl)-6-nitroquinoline

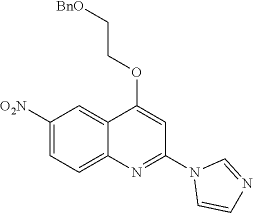 [SMILES: O=[N+]([O-])c1ccc2nc(-n3ccnc3)cc(OCCOCc3ccccc3)c2c1]

4-(2-(Benzyloxy)ethoxy)-2-(1H-imidazol-1-yl)-6-nitroquinoline was prepared using the procedure followed for preparation of 4-ethoxy-2-(4-methyl-1H-imidazol-1-yl)-6-nitroquinoline. The crude was purified by silica gel (100-200 mesh) flash column chromatography (2% MeOH in CH₂Cl₂) to give the title product as off-white solid (0.3 g, yield: 61%). LCMS (ESI+): m/z calcd. for C₂₁H₁₉N₄O₄ [M+H]⁺, 391; found, 391.

### 2. Synthesis of 2-((2-(1H-imidazol-1-yl)-6-nitroquinolin-4-yl)oxy)ethan-1-ol

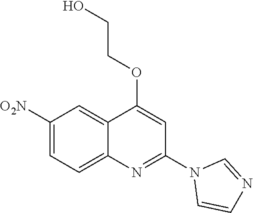 [SMILES: O=[N+]([O-])c1ccc2nc(-n3ccnc3)cc(OCCO)c2c1]

2-((2-(1H-imidazol-1-yl)-6-nitroquinolin-4-yl)oxy)ethan-1-ol was prepared using the procedure followed for preparation of 2-((6-nitro-2-(thiazol-5-yl) quinolin-4-yl) oxy) ethan-1-ol. The product was obtained as yellow solid (0.2 g, yield: 80%). LCMS (ESI+): m/z calcd. for C₁₄H₁₃N₄O₄ [M+H]⁺, 301; found, 301.

### 3. Synthesis of 2-((6-amino-2-(1H-imidazol-1-yl)quinolin-4-yl)oxy)ethan-1-ol

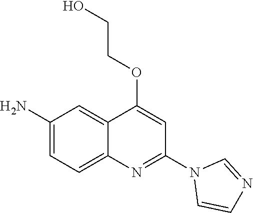 [SMILES: Nc1ccc2nc(-n3ccnc3)cc(OCCO)c2c1]

2-((6-amino-2-(1H-imidazol-1-yl)quinolin-4-yl)oxy)ethan-1-ol was prepared using the procedure followed for preparation of 4-(2-methoxyethoxy)-2-(thiazol-5-yl) quinolin-6-amine. The desired product was obtained as a brown solid (0.22 g, crude). LCMS (ESI+): m/z calcd. for C₁₄H₁₅N₄O₂ [M+H]⁺, 271; found, 271.

### 4. Synthesis of N-(4-(2-hydroxyethoxy)-2-(1H-imidazol-1-yl)quinolin-6-yl)oxetane-3-carboxamide

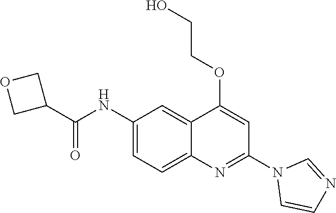 [SMILES: O=C(Nc1ccc2nc(-n3ccnc3)cc(OCCO)c2c1)C1COC1]

N-(4-(2-hydroxyethoxy)-2-(1H-imidazol-1-yl)quinolin-6-yl)oxetane-3-carboxamide was prepared following the procedure of N-(4-(2-methoxyethoxy)-2-(thiazol-5-yl)quinolin-6-yl)oxetane-3-carboxamide as off white solid (0.08 g, yield: 5%). HPLC purity: 97.71%. LCMS (ESI+): m/z calcd. for C₁₈H₁₉N₄O₄ [M+H]⁺, 355; found, 355. ¹H NMR (400 MHz, DMSO-d₆, δ): 10.30 (s, 1H), 8.71 (s, 1H), 8.49 (d, J=1.47 Hz, 1H), 8.13 (s, 1H), 7.98 (dd, J=9.05, 1.71 Hz, 1H), 7.85 (d, J=8.80 Hz, 1H), 7.41 (s, 1H), 7.15 (s, 1H), 5.06 (br t, J=4.89 Hz, 1H), 4.74-4.71 (m, 4H), 4.44 (br t, J=4.40 Hz, 2H), 4.03-4.00 (m, 1H), 3.92 (q, J=4.40 Hz, 2H).

### Example 43: N-(4-cyclopropoxy-2-(4-methyl-1H-imidazol-1-yl)quinolin-6-yl)oxetane-3-carboxamide

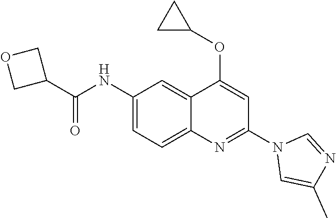 [SMILES: Cc1cn(-c2cc(OC3CC3)c3cc(NC(=O)C4COC4)ccc3n2)cn1]

N-(4-cyclopropoxy-2-(4-methyl-1H-imidazol-1-yl)quinolin-6-yl)oxetane-3-carboxamide was prepared following the procedure of Example 2. HPLC purity: 99.74%. LCMS (ESI+): m/z calcd. for C₂₀H₂₁N₄O₃ [M+H]⁺, 365; found, 365. ¹H NMR (400 MHz, DMSO-d₆, δ): 10.35 (s, 1H), 8.65 (s, 1H), 8.49 (s, 1H), 7.95 (s, 1H), 7.89 (d, J=8.4 Hz, 2H), 7.63 (s, 1H), 4.79 (d, J=7.4 Hz, 4H), 4.39 (s, 1H), 4.11-4.03 (m, 1H), 2.28 (s, 3H), 1.09 (d, J=7.1 Hz, 2H), 0.94 (brs, 2H).

### Example 44: N-(2-(4-chloro-1H-imidazol-1-yl)-4-ethoxyquinolin-6-yl)oxetane-3-carboxamide

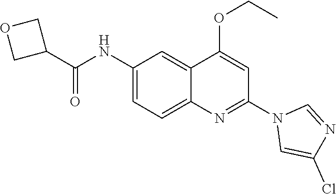 [SMILES: CCOc1cc(-n2cnc(Cl)c2)nc2ccc(NC(=O)C3COC3)cc12]

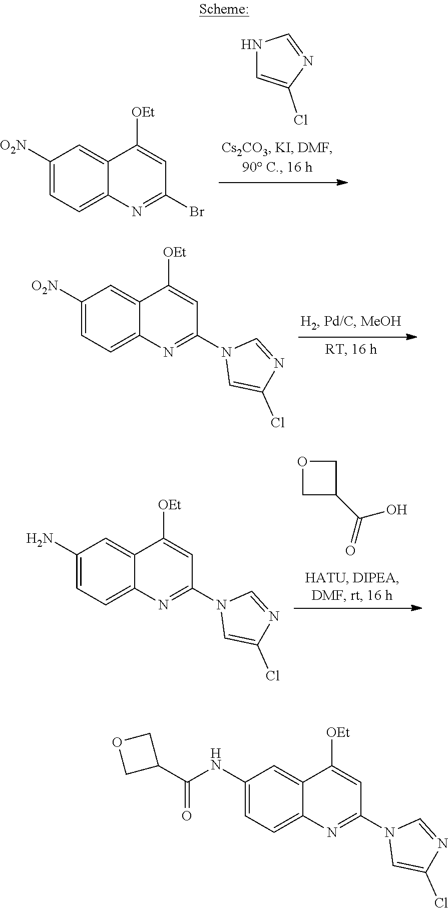 [SMILES: CCOc1cc(Br)nc2ccc([N+](=O)[O-])cc12 ; Clc1c[nH]cn1 ; CCOc1cc(-n2cnc(Cl)c2)nc2ccc([N+](=O)[O-])cc12 ; CCOc1cc(-n2cnc(Cl)c2)nc2ccc(N)cc12 ; O=C(O)C1COC1 ; CCOc1cc(-n2cnc(Cl)c2)nc2ccc(NC(=O)C3COC3)cc12]

### 1. Synthesis of 2-(4-chloro-1H-imidazol-1-yl)-4-ethoxy-6-nitroquinoline

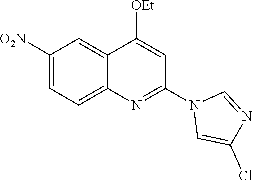 [SMILES: CCOc1cc(-n2cnc(Cl)c2)nc2ccc([N+](=O)[O-])cc12]

2-(4-Chloro-1H-imidazol-1-yl)-4-ethoxy-6-nitroquinoline was prepared using the procedure followed for preparation of 4-ethoxy-2-(4-methyl-1H-imidazol-1-yl)-6-nitroquinoline. The crude compound was triturated with diethylether and n-pentane to afford the title compound as off-white solid (0.21 g, yield: 65%). LCMS (ESI+): m/z calcd. for C₁₄H₁₂ClN₄O₃ [M+H]⁺, 319; found, 319. ¹H NMR (400 MHz, DMSO-d₆, δ): 8.89 (d, J=2.45 Hz, 1H), 8.81 (s, 1H), 8.50 (dd, J=9.05, 2.69 Hz, 1H), 8.34 (s, 1H), 8.06 (d, J=9.29 Hz, 1H), 7.59 (s, 1H), 4.53 (q, J=7.01 Hz, 2H), 1.56 (t, J=6.85 Hz, 3H).

### 2. Synthesis of 2-(4-chloro-1H-imidazol-1-yl)-4-ethoxyquinolin-6-amine

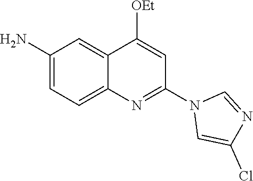 [SMILES: CCOc1cc(-n2cnc(Cl)c2)nc2ccc(N)cc12]

2-(4-Chloro-1H-imidazol-1-yl)-4-ethoxyquinolin-6-amine was prepared using the procedure followed for preparation of 4-ethoxy-2-(4-methyl-1H-imidazol-1-yl) quinolin-6-amine as brown solid (0.15 g, yield: 79%). LCMS (ESI+): m/z calcd. for C₁₄H₁₄ClN₄O [M+H]⁺, 289; found, 289. ¹H NMR (400 MHz, DMSO-d₆, δ): 8.58 (br s, 1H), 8.15 (br s, 1H), 7.61 (br d, J=7.83 Hz, 1H), 7.00-7.28 (m, 3H), 5.84 (br s, 2H), 4.37 (q, J=7.83, Hz, 2H), 1.49 (t, J=8.31 Hz, 3H).

### 3. Synthesis of N-(2-(4-chloro-1H-imidazol-1-yl)-4-ethoxyquinolin-6-yl)oxetane-3-carboxamide

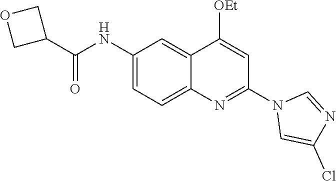 [SMILES: CCOc1cc(-n2cnc(Cl)c2)nc2ccc(NC(=O)C3COC3)cc12]

N-(2-(4-chloro-1H-imidazol-1-yl)-4-ethoxyquinolin-6-yl)oxetane-3-carboxamide was prepared following the procedure of N-(4-ethoxy-2-(4-methyl-1H-imidazol-1-yl)quinolin-6-yl)oxetane-3-carboxamide as a white solid (0.08 g, yield: 41%). HPLC purity: 96.86%. LCMS (ESI+): m/z calcd. for C₁₈H₁₈ClN₄O₃[M+H]⁺, 373; found, 373. ¹H NMR (400 MHz, DMSO-d₆, δ): 10.29 (br s, 1H), 8.67 (br s, 1H), 8.52 (br s, 1H), 8.23 (br s, 1H), 7.77-7.99 (m, 2H), 7.38 (br s, 1H), 4.73 (d, J=5.38 Hz, 4H), 4.45 (d, J=5.00 Hz, 2H), 4.03-3.99 (m, 1H), 1.52 (t, J=6.63 Hz, 3H).

### Example 45: N-(4-ethoxy-2-(4H-1,2,4-triazol-4-yl)quinolin-6-yl)oxetane-3-carboxamide

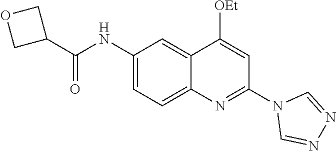 [SMILES: CCOc1cc(-n2cnnc2)nc2ccc(NC(=O)C3COC3)cc12]

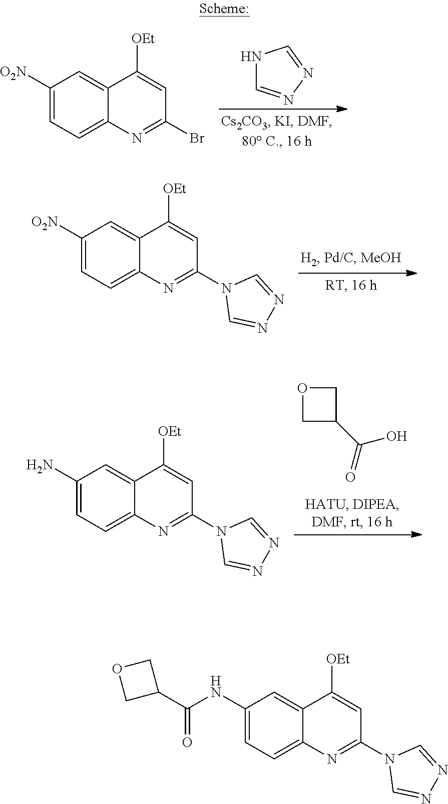 [SMILES: CCOc1cc(Br)nc2ccc([N+](=O)[O-])cc12 ; c1nnc[nH]1 ; CCOc1cc(-n2cnnc2)nc2ccc([N+](=O)[O-])cc12 ; CCOc1cc(-n2cnnc2)nc2ccc(N)cc12 ; O=C(O)C1COC1 ; CCOc1cc(-n2cnnc2)nc2ccc(NC(=O)C3COC3)cc12]

### 1. Synthesis of 4-ethoxy-6-nitro-2-(4H-1,2,4-triazol-4-yl)quinoline

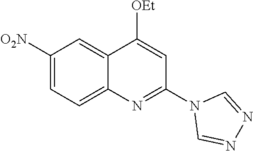 [SMILES: CCOc1cc(-n2cnnc2)nc2ccc([N+](=O)[O-])cc12]

4-Ethoxy-6-nitro-2-(4H-1,2,4-triazol-4-yl)quinoline was prepared using the procedure followed for preparation of 4-ethoxy-2-(4-methyl-1H-imidazol-1-yl)-6-nitroquinoline. The crude was triturated with diethylether and n-pentane to afford the title compound as a white solid (0.2 g, yield: 69%). LCMS (ESI+): m/z calcd. for C₁₃H₁₂N₅O₃ [M+H]⁺, 286; found, 286. ¹H NMR (400 MHz, DMSO-d₆, δ): 9.61 (s, 1H), 8.96 (br s, 1H), 8.55 (br d, J=7.83 Hz, 1H), 8.43 (s, 1H), 8.13 (br d, J=9.29 Hz, 1H), 7.62 (s, 1H), 4.57 (t, J=6.36 Hz, 2H), 1.57 (br t, J=6.85 Hz, 3H).

### 2. Synthesis of 4-ethoxy-2-(4H-1,2,4-triazol-4-yl)quinolin-6-amine

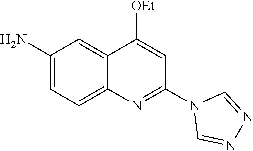 [SMILES: CCOc1cc(-n2cnnc2)nc2ccc(N)cc12]

4-Ethoxy-2-(4H-1,2,4-triazol-4-yl)quinolin-6-amine was prepared using the procedure followed for preparation of 4-ethoxy-2-(4-methyl-1H-imidazol-1-yl) quinolin-6-amine. The crude compound was triturated with diethyl ether and n-pentane to afford the title compound as white solid (0.14 g, yield: 78%). LCMS (ESI+): m/z calcd. for C₁₃H₁₄N₅O [M+H]⁺, 256; found, 256. ¹H NMR (400 MHz, DMSO-d₆, δ): 9.35 (s, 1H), 8.28 (s, 1H), 7.64 (d, J=8.80 Hz, 1H), 7.27 (s, 1H), 7.18 (dd, J=8.80, 2.45 Hz, 1H), 7.11 (d, J=2.45 Hz, 1H), 5.72 (s, 2H), 4.36 (q, J=6.85 Hz, 2H), 1.50 (t, J=6.85 Hz, 3H).

### 3. Synthesis of N-(4-ethoxy-2-(4H-1,2,4-triazol-4-yl)quinolin-6-yl)oxetane-3-carboxamide

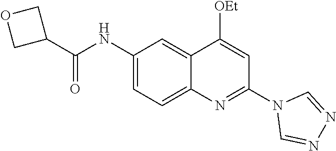 [SMILES: CCOc1cc(-n2cnnc2)nc2ccc(NC(=O)C3COC3)cc12]

N-(4-ethoxy-2-(4H-1,2,4-triazol-4-yl)quinolin-6-yl)oxetane-3-carboxamide was prepared following the procedure of N-(4-ethoxy-2-(4-methyl-1H-imidazol-1-yl)quinolin-6-yl)oxetane-3-carboxamide as a white solid (0.058 g, yield: 26%). HPLC purity: 97.70%. LCMS (ESI+): m/z calcd. for C₁₇H₁₈N₅O₃ [M+H]⁺, 340; found, 340. ¹H NMR (400 MHz, DMSO-d₆, δ): 10.33 (s, 1H), 9.46 (s, 1H), 8.55 (d, J=2.25 Hz, 1H), 8.34 (s, 1H), 7.96-8.04 (m, 1H), 7.88-7.93 (m, 1H), 7.43 (s, 1H), 4.76-4.72 (m, 4H), 4.45 (q, J=6.96 Hz, 2H), 4.04-4.01 (m, 1H), 1.53 (t, J=6.94 Hz, 3H).

### Example 46: N-(2-(4-cyano-1H-imidazol-1-yl)-4-ethoxyquinolin-6-yl)oxetane-3-carboxamide

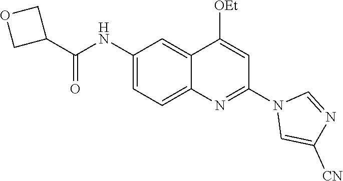 [SMILES: CCOc1cc(-n2cnc(C#N)c2)nc2ccc(NC(=O)C3COC3)cc12]

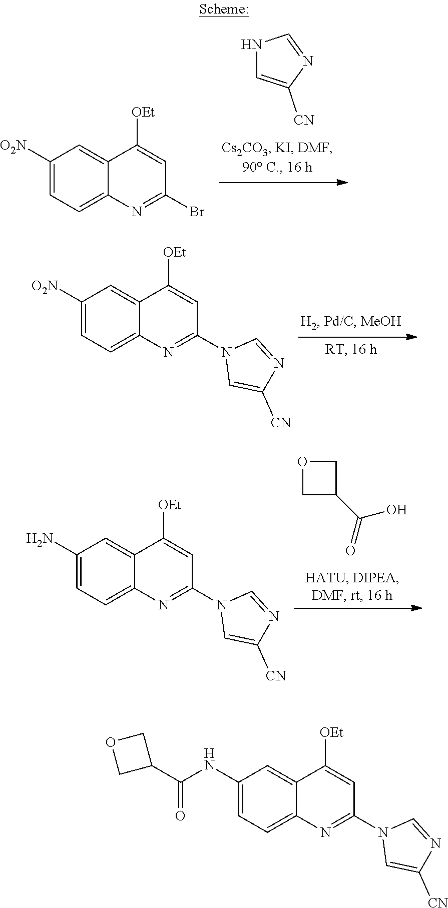 [SMILES: CCOc1cc(Br)nc2ccc([N+](=O)[O-])cc12 ; N#Cc1c[nH]cn1 ; CCOc1cc(-n2cnc(C#N)c2)nc2ccc([N+](=O)[O-])cc12 ; CCOc1cc(-n2cnc(C#N)c2)nc2ccc(N)cc12 ; O=C(O)C1COC1 ; CCOc1cc(-n2cnc(C#N)c2)nc2ccc(NC(=O)C3COC3)cc12]

### 1. Synthesis of 1-(4-ethoxy-6-nitroquinolin-2-yl)-1H-imidazole-4-carbonitrile

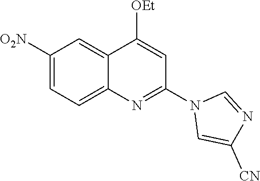 [SMILES: CCOc1cc(-n2cnc(C#N)c2)nc2ccc([N+](=O)[O-])cc12]

1-(4-Ethoxy-6-nitroquinolin-2-yl)-1H-imidazole-4-carbonitrile was prepared using the procedure followed for preparation of 4-ethoxy-2-(4-methyl-1H-imidazol-1-yl)-6-nitroquinoline. The crude was triturated with acetonitrile (25 mL) to give the title product as yellow solid (0.19 g, yield: 61%). ¹H NMR (400 MHz, DMSO-d₆, δ): 8.89 (d, J=2.45 Hz, 1H), 8.80 (s, 1H), 8.49 (dd, J=9.29, 2.45 Hz, 1H), 8.39 (s, 1H), 8.05 (d, J=9.29 Hz, 1H), 7.58 (s, 1H), 4.52 (q, J=7.17 Hz, 2H), 1.56 (t, J=6.85 Hz, 3H).

### 2. Synthesis of 1-(6-amino-4-ethoxyquinolin-2-yl)-1H-imidazole-4-carbonitrile

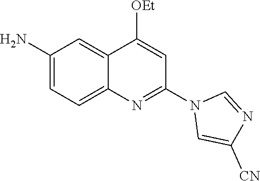 [SMILES: CCOc1cc(-n2cnc(C#N)c2)nc2ccc(N)cc12]

1-(6-Amino-4-ethoxyquinolin-2-yl)-1H-imidazole-4-carbonitrile was prepared using the procedure followed for preparation of 4-ethoxy-2-(4-methyl-1H-imidazol-1-yl) quinolin-6-amine. The title product was obtained as a yellow solid (0.1 g, yield: 58%). LCMS (ESI+): m/z calcd. for C₁₅H₁₄N₅O [M+H]⁺, 280; found, 280. ¹H NMR (400 MHz, DMSO-d₆, δ): 8.97 (s, 1H), 8.80 (s, 1H), 7.62 (br d, J=8.80 Hz, 1H), 7.29 (s, 1H), 7.17 (t, J=1.47 Hz, 1H), 7.09 (d, J=1.47 Hz, 1H), 5.64-5.82 (m, 2H), 4.38 (q, J=7.01 Hz, 2H), 1.50 (br t, J=6.85 Hz, 3H).

### 3. Synthesis of N-(2-(4-cyano-1H-imidazol-1-yl)-4-ethoxyquinolin-6-yl)oxetane-3-carboxamide

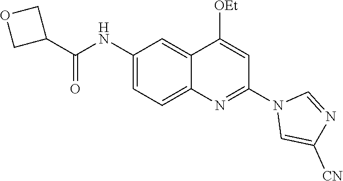 [SMILES: CCOc1cc(-n2cnc(C#N)c2)nc2ccc(NC(=O)C3COC3)cc12]

N-(2-(4-cyano-1H-imidazol-1-yl)-4-ethoxyquinolin-6-yl)oxetane-3-carboxamide was prepared following the procedure of N-(4-ethoxy-2-(4-methyl-1H-imidazol-1-yl)quinolin-6-yl)oxetane-3-carboxamide as a white solid (0.012 g, yield: 10%). HPLC purity: 98.02%. LCMS (ESI+): m/z calcd. for C₁₉H₁₈N₅O₃ [M+H]⁺, 364; found, 364. ¹H NMR (400 MHz, DMSO-d₆, δ): 10.33 (s, 1H), 9.06 (d, J=1.25 Hz, 1H), 8.90 (d, J=1.25 Hz, 1H), 8.56 (d, J=2.25 Hz, 1H), 7.94-8.01 (m, 1H), 7.89 (d, J=9.01 Hz, 1H), 7.48 (s, 1H), 4.76-4.72 (m, 4H), 4.47 (q, J=7.00 Hz, 2H), 4.04-4.01 (m, 1H), 1.54 (t, J=6.94 Hz, 3H).

### Example 47: N-(2-(4-(tert-butyl)-1H-imidazol-1-yl)-4-ethoxyquinolin-6-yl)oxetane-3-carboxamide

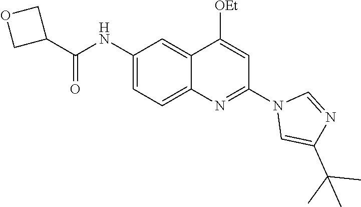 [SMILES: CCOc1cc(-n2cnc(C(C)(C)C)c2)nc2ccc(NC(=O)C3COC3)cc12]

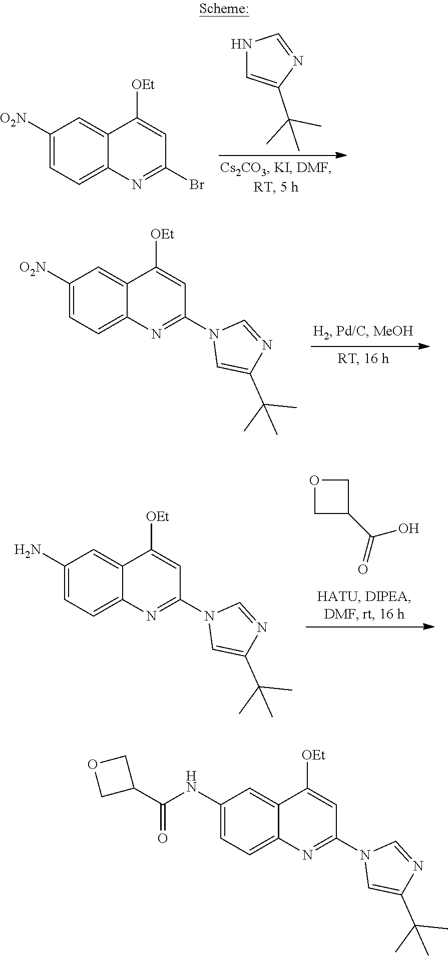

### 1. Synthesis of 2-(4-(tert-butyl)-1H-imidazol-1-yl)-4-ethoxy-6-nitroquinoline

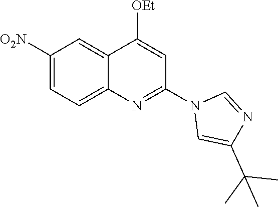 [SMILES: CCOc1cc(-n2cnc(C(C)(C)C)c2)nc2ccc([N+](=O)[O-])cc12]

2-(4-(tert-Butyl)-1H-imidazol-1-yl)-4-ethoxy-6-nitroquinoline was prepared using the procedure followed for preparation of 4-ethoxy-2-(4-methyl-1H-imidazol-1-yl)-6-nitroquinoline. The title product was obtained as a pale green solid (0.2 g, yield: 86%). LCMS (ESI+): m/z calcd. for C₁₈H₂₁N₄O₃ [M+H]⁺, 341; found, 341.

### 2. Synthesis of 2-(4-(tert-butyl)-1H-imidazol-1-yl)-4-ethoxyquinolin-6-amine

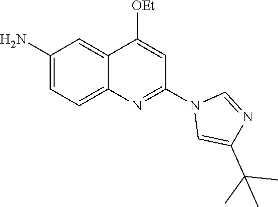 [SMILES: CCOc1cc(-n2cnc(C(C)(C)C)c2)nc2ccc(N)cc12]

2-(4-(tert-Butyl)-1H-imidazol-1-yl)-4-ethoxyquinolin-6-amine was prepared using the procedure followed for preparation of 4-ethoxy-2-(4-methyl-1H-imidazol-1-yl) quinolin-6-amine. The title product was obtained as a pale green solid (0.15 g, yield: 82%). LCMS (ESI+): m/z calcd. for C₁₈H₂₃N₄O [M+H]⁺, 311; found, 311. ¹H NMR (400 MHz, DMSO-d₆, δ): 8.47 (s, 1H), 7.70 (s, 1H), 7.58 (d, J=8.80 Hz, 1H), 7.04-7.18 (m, 3H), 5.58 (s, 2H), 4.37 (q, J=6.36 Hz, 2H), 1.49 (br t, J=6.85 Hz, 3H), 1.28 (s, 9H).

### 3. Synthesis of N-(2-(4-(tert-butyl)-1H-imidazol-1-yl)-4-ethoxyquinolin-6-yl)oxetane-3-carboxamide

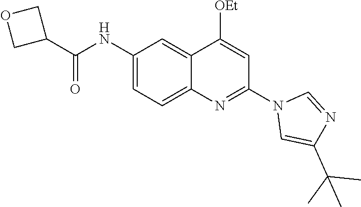 [SMILES: CCOc1cc(-n2cnc(C(C)(C)C)c2)nc2ccc(NC(=O)C3COC3)cc12]

N-(2-(4-(tert-butyl)-1H-imidazol-1-yl)-4-ethoxyquinolin-6-yl)oxetane-3-carboxamide was prepared following the procedure of N-(4-ethoxy-2-(4-methyl-1H-imidazol-1-yl)quinolin-6-yl)oxetane-3-carboxamide as an off-white solid (0.06 g, yield: 31%). HPLC purity: 97.48%. LCMS (ESI+): m/z calcd. for C₂₂H₂₇N₄O₃ [M+H]⁺, 395; found, 395. ¹H NMR (400 MHz, DMSO-d₆, δ): 10.26 (s, 1H), 8.58 (d, J=1.25 Hz, 1H), 8.48 (d, J=2.25 Hz, 1H), 7.91-7.96 (m, 1H), 7.83 (d, J=9.01 Hz, 1H), 7.78 (d, J=1.25 Hz, 1H), 7.34 (s, 1H), 4.74-4.72 (m, 4H), 4.46 (q, J=6.92 Hz, 2H), 4.02-4.00 (m, 1H), 1.53 (t, J=7.00 Hz, 3H), 1.29 (s, 9H).

### Example 48: 2-cyano-N-(4-(((S)-1-methoxypropan-2-yl)oxy)-2-(thiazol-5-yl)quinolin-6-yl)propanamide

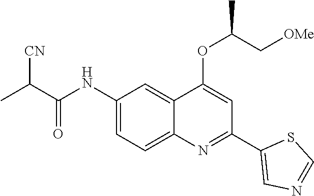 [SMILES: COC[C@H](C)Oc1cc(-c2cncs2)nc2ccc(NC(=O)C(C)C#N)cc12]

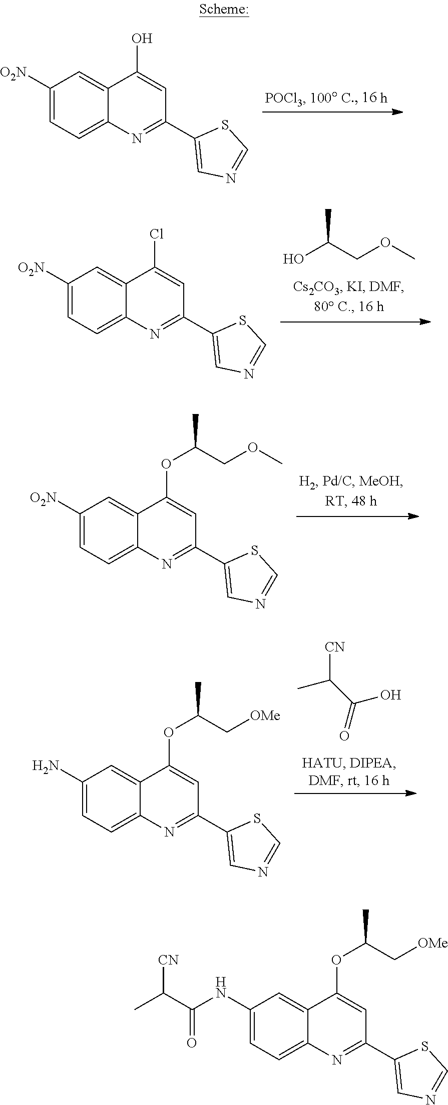

### 1. Synthesis of 5-(4-chloro-6-nitroquinolin-2-yl)thiazole

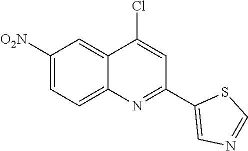 [SMILES: O=[N+]([O-])c1ccc2nc(-c3cncs3)cc(Cl)c2c1]

5-(4-Chloro-6-nitroquinolin-2-yl)thiazole was prepared using the procedure followed for preparation of 2,4-dichloro-6-nitroquinoline. The crude was triturated with Et₂O to afford the desired product as brown solid (1 g, crude was used for the next step without purification). LCMS (ESI+): m/z calcd. for C₁₂H₇ClN₃O₂S [M+H]⁺, 292; found, 292. ¹H NMR (400 MHz, DMSO-d₆, δ): 9.34 (s, 1H), 9.00 (s, 1H), 8.92 (d, J=2.0 Hz, 1H), 8.71 (s, 1H), 8.53 (dd, J=9.0, 2.2 Hz, 1H), 8.22 (d, J=9.3 Hz, 1H).

### 2. Synthesis of (S)-5-(4-((1-methoxypropan-2-yl)oxy)-6-nitroquinolin-2-yl)thiazole

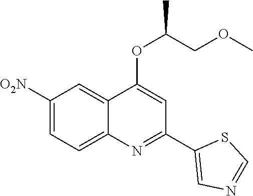 [SMILES: COC[C@H](C)Oc1cc(-c2cncs2)nc2ccc([N+](=O)[O-])cc12]

To a stirred solution of 5-(4-chloro-6-nitroquinolin-2-yl)thiazole (0.65 g, 2.22 mmol) in DMF (18 mL) was added Cs₂CO₃ (1.81 g, 5.55 mmol), KI (0.36 g, 0.21 mmol) and (S)-1-methoxypropan-2-ol (0.5 g, 5.57 mmol) at RT. The reaction was stirred at 80° C. for 16 h in a sealed tube. Then, the reaction mixture was diluted with ice-cold water (100 mL), extracted with EtOAc (2×50 mL). The combined organic extracts were dried over anhydrous sodium sulfate, filtered and concentrated in vacuo to afford the title product (0.38 g, 49%) as yellow solid. LCMS (ESI+): m/z calcd. for C₁₆H₁₆N₃O₄S [M+H]⁺, 346; found, 346.

### 3. Synthesis of(S)-4-((1-methoxypropan-2-yl)oxy)-2-(thiazol-5-yl)quinolin-6-amine

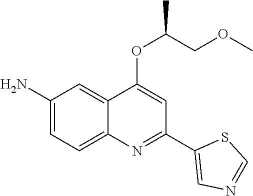 [SMILES: COC[C@H](C)Oc1cc(-c2cncs2)nc2ccc(N)cc12]

(S)-4-((1-Methoxypropan-2-yl)oxy)-2-(thiazol-5-yl)quinolin-6-amine was prepared using the procedure followed for preparation of 4-(2-methoxyethoxy)-2-(thiazol-5-yl) quinolin-6-amine as brown solid (0.32 g, yield: 92%). ¹H NMR (400 MHz, DMSO-d₆, δ): 9.07 (br s, 1H), 8.66 (s, 1H), 7.58 (br d, J=8.3 Hz, 1H), 7.46 (s, 1H), 7.16-6.98 (m, 3H), 5.65 (br s, 1.6H), 5.14-5.11 (m, 0.4H), 3.62 (br s, 2H), 3.33 (br s, 3H), 1.34 (br d, J=5.9 Hz, 3H).

### 4. Synthesis of 2-cyano-N-(4-(((S)-1-methoxypropan-2-yl)oxy)-2-(thiazol-5-yl)quinolin-6-yl)propanamide

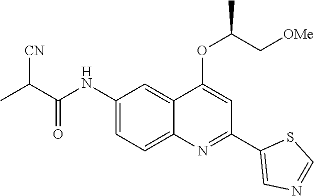 [SMILES: COC[C@H](C)Oc1cc(-c2cncs2)nc2ccc(NC(=O)C(C)C#N)cc12]

2-Cyano-N-(4-(((S)-1-methoxypropan-2-yl)oxy)-2-(thiazol-5-yl)quinolin-6-yl)propanamide was prepared following the procedure of N-(4-(2-methoxyethoxy)-2-(thiazol-5-yl)quinolin-6-yl)oxetane-3-carboxamide as an off-white solid (0.08 g, yield: 42%). HPLC purity: 96.91%. LCMS (ESI+): m/z calcd. for C₂₀H₂₁N₄O₃S [M+H]⁺, 397; found, 397. ¹H NMR (400 MHz, DMSO-d₆, δ): 10.72 (s, 1H), 9.18 (s, 1H), 8.84 (s, 1H), 8.38 (s, 1H), 7.99-7.83 (m, 3H), 7.70 (s, 1H), 5.31-5.21 (m, 1H), 4.00 (q, J=6.8 Hz, 1H), 3.75-3.62 (m, 1H), 3.36 (s, 3H), 1.55 (br d, J=6.8 Hz, 3H), 1.39 (br d, J=5.9 Hz, 3H).

### Example 49: 2-cyano-N-(4-(((R)-1-methoxypropan-2-yl)oxy)-2-(thiazol-5-yl)quinolin-6-yl)propanamide

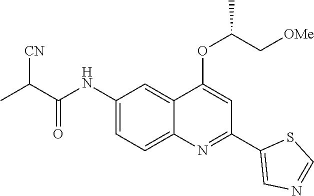 [SMILES: COC[C@@H](C)Oc1cc(-c2cncs2)nc2ccc(NC(=O)C(C)C#N)cc12]

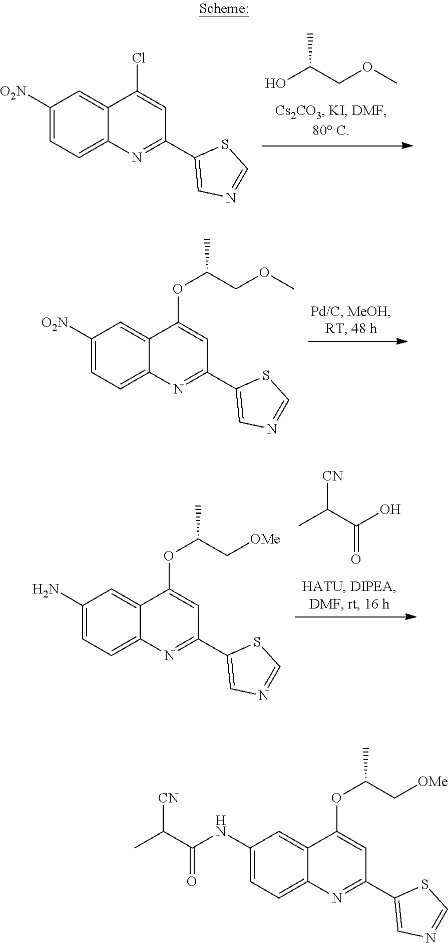

### 1. Synthesis of (R)-5-(4-((1-methoxypropan-2-yl)oxy)-6-nitroquinolin-2-yl)thiazole

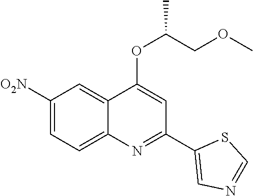 [SMILES: COC[C@@H](C)Oc1cc(-c2cncs2)nc2ccc([N+](=O)[O-])cc12]

(R)-5-(4-((1-Methoxypropan-2-yl)oxy)-6-nitroquinolin-2-yl)thiazole was prepared using the procedure followed for preparation of (S)-5-(4-((1-methoxypropan-2-yl)oxy)-6-nitroquinolin-2-yl)thiazole. The crude was purified by silica gel column chromatography (20-30% EtOAc/heptane) to afford the desired product as a yellow solid (0.4 g, yield: 45%). LCMS (ESI+): m/z calcd. for C₁₆H₁₆N₃O₄S [M+H]⁺, 346; found, 346. ¹H NMR (400 MHz, DMSO-d₆, δ): 9.30 (s, 1H), 9.01 (s, 1H), 8.90 (br s, 1H), 8.45 (br d, J=6.8 Hz, 1H), 8.09 (br d, J=9.3 Hz, 1H), 7.93 (s, 1H), 5.42-5.29 (m, 1H), 3.83-3.64 (m, 2H), 3.36 (s, 3H), 1.43 (br d, J=6.4 Hz, 3H).

### 2. Synthesis of (R)-4-((1-methoxypropan-2-yl)oxy)-2-(thiazol-5-yl)quinolin-6-amine

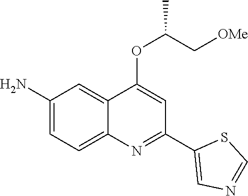 [SMILES: COC[C@@H](C)Oc1cc(-c2cncs2)nc2ccc(N)cc12]

(R)-4-((1-Methoxypropan-2-yl)oxy)-2-(thiazol-5-yl)quinolin-6-amine was prepared using the procedure followed for preparation of 4-(2-methoxyethoxy)-2-(thiazol-5-yl) quinolin-6-amine as yellow solid (0.45 g, crude was used for the next step without purification). LCMS (ESI+): m/z calcd. for C₁₆H₁₈N₃O₂S [M+H]⁺, 316; found, 316.

### 3. Synthesis of 2-cyano-N-(4-(((R)-1-methoxypropan-2-yl)oxy)-2-(thiazol-5-yl)quinolin-6-yl)propanamide

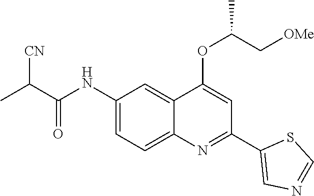 [SMILES: COC[C@@H](C)Oc1cc(-c2cncs2)nc2ccc(NC(=O)C(C)C#N)cc12]

2-Cyano-N-(4-(((R)-1-methoxypropan-2-yl)oxy)-2-(thiazol-5-yl)quinolin-6-yl)propanamide was prepared following the procedure of Example 1 as a pale yellow solid (0.02 g, yield: 10%). HPLC purity: 95.60%. LCMS (ESI+): m/z calcd. for C₂₀H₂₁N₄O₃S [M+H]⁺, 397; found, 397. ¹H NMR (400 MHz, DMSO-d₆, δ): 10.71 (br s, 1H), 9.19 (s, 1H), 8.84 (s, 1H), 8.41-8.37 (m, 1H), 7.96-7.87 (m, 2H), 7.70 (s, 1H), 5.33-5.17 (m, 1H), 4.01 (q, J=7.2 Hz, 1H), 3.74-3.64 (m, 2H), 3.37 (s, 3H), 1.56 (d, J=7.3 Hz, 3H), 1.40 (d, J=6.3 Hz, 3H).

### Example 50: (S)-2-methoxy-N-(4-(((S)-1-methoxypropan-2-yl)oxy)-2-(thiazol-5-yl)quinolin-6-yl)propanamide

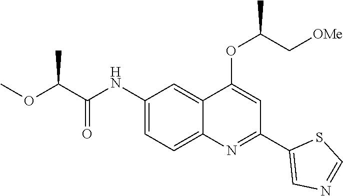 [SMILES: COC[C@H](C)Oc1cc(-c2cncs2)nc2ccc(NC(=O)[C@H](C)OC)cc12]

(S)-2-Methoxy-N-(4-(((S)-1-methoxypropan-2-yl)oxy)-2-(thiazol-5-yl)quinolin-6-yl)propanamide was prepared following the procedure of Example 1 as an off white solid (0.03 g, yield: 14%). HPLC purity: 99.91%. LCMS (ESI+): m/z calcd. for C₂₀H₂₄N₃O₄S [M+H]⁺, 402; found, 402. ¹H NMR (400 MHz, DMSO-d₆, δ): 10.18 (s, 1H), 9.17 (s, 1H), 8.82 (s, 1H), 8.52 (d, J=2.3 Hz, 1H), 8.04 (dd, J=9.1, 2.4 Hz, 1H), 7.85 (d, J=9.0 Hz, 1H), 7.67 (s, 1H), 5.32-5.15 (m, 1H), 3.93 (q, J=6.7 Hz, 1H), 3.72-3.65 (m, 2H), 3.36 (s, 3H), 3.34 (s, 3H), 1.39 (d, J=6.1 Hz, 3H), 1.36 (d, J=6.6 Hz, 3H).

### Example 51: (S)-2-methoxy-N-(4-(((R)-1-methoxypropan-2-yl)oxy)-2-(thiazol-5-yl)quinolin-6-yl)propanamide

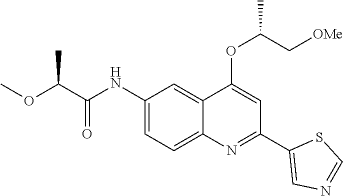 [SMILES: COC[C@@H](C)Oc1cc(-c2cncs2)nc2ccc(NC(=O)[C@H](C)OC)cc12]

(S)-2-Methoxy-N-(4-(((R)-1-methoxypropan-2-yl)oxy)-2-(thiazol-5-yl)quinolin-6-yl)propanamide was prepared following the procedure of Example 1 as a yellow solid (0.1 g, yield: 40%). HPLC purity: 98.45%. LCMS (ESI+): m/z calcd. for C₂₀H₂₄N₃O₄S [M+H]⁺, 402; found, 402. ¹H NMR (400 MHz, DMSO-d₆) δ ¹H NMR (400 MHz, DMSO-d₆, δ): 10.19 (s, 1H), 9.19 (s, 1H), 8.83 (s, 1H), 8.53 (d, J=2.3 Hz, 1H), 8.06 (dd, J=2.4, 9.1 Hz, 1H), 7.86 (d, J=9.1 Hz, 1H), 7.67 (s, 1H), 5.33-5.19 (m, 1H), 3.93 (q, J=6.7 Hz, 1H), 3.76-3.61 (m, 2H), 3.36 (s, 3H), 3.34 (s, 3H), 1.40 (d, J=6.1 Hz, 3H), 1.36 (d, J=6.6 Hz, 3H).

### Example 52: (R)—N-(4-((1-methoxypropan-2-yl)oxy)-2-(thiazol-5-yl)quinolin-6-yl)oxetane-3-carboxamide

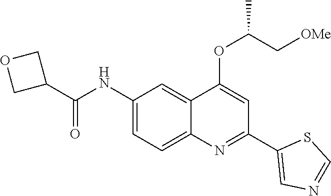 [SMILES: COC[C@@H](C)Oc1cc(-c2cncs2)nc2ccc(NC(=O)C3COC3)cc12]

(R)—N-(4-((1-methoxypropan-2-yl)oxy)-2-(thiazol-5-yl)quinolin-6-yl)oxetane-3-carboxamide was prepared following the procedure of Example 1 as a white solid (0.065 g, yield: 25%). HPLC purity: 98.81%. LCMS (ESI+): m/z calcd. for C₂₀H₂₂N₃O₄S [M+H]⁺, 400; found, 400. ¹H NMR (400 MHz, DMSO-d₆, δ): 10.27 (s, 1H), 9.17 (s, 1H), 8.82 (s, 1H), 8.42 (d, J=2.3 Hz, 1H), 7.96 (dd, J=9.1, 2.4 Hz, 1H), 7.86 (d, J=9.1 Hz, 1H), 7.67 (s, 1H), 5.32-5.17 (m, 1H), 4.75-4.72 (m, 4H), 4.04-4.00 (m, 1H), 3.75-3.62 (m, 2H), 3.36 (s, 3H), 1.39 (d, J=6.3 Hz, 3H).

### Example 53: N-(4-(2-methoxyethoxy)-2-(1-methyl-1H-pyrazol-4-yl)quinolin-6-yl)oxetane-3-carboxamide

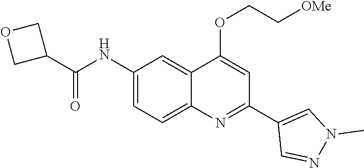 [SMILES: COCCOc1cc(-c2cnn(C)c2)nc2ccc(NC(=O)C3COC3)cc12]

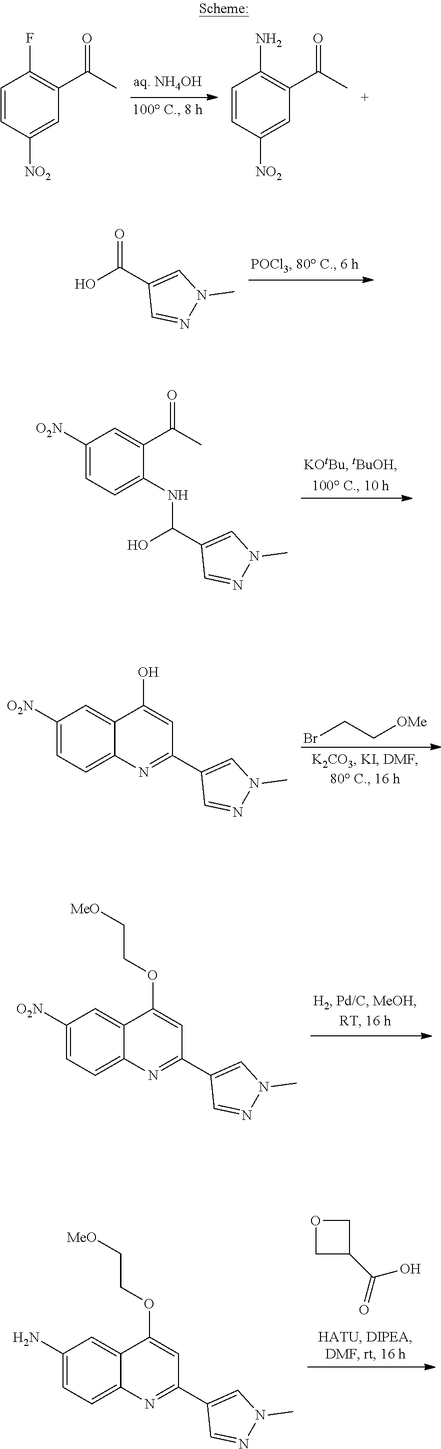

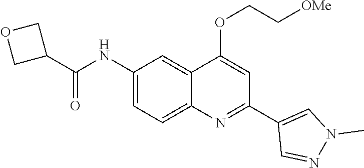 [SMILES: COCCOc1cc(-c2cnn(C)c2)nc2ccc(NC(=O)C3COC3)cc12]

### 1. Synthesis of 1-(2-amino-5-nitrophenyl)ethan-1-one

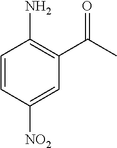

A mixture of 1-(2-fluoro-5-nitrophenyl)ethan-1-one (28 g, 152.88 mmol) and NH₄OH (25%, 230 mL) was heated in an autoclave at 100° C. and stirred for 8 h. Then, the reaction mixture was filtered, and washed with ice-cold water (100 mL) and dried under vacuum. The crude was triturated with acetonitrile (100 mL) and dried in vacuo to 1-(2-amino-5-nitrophenyl)ethan-1-one (17 g, 75%) as yellow solid. ¹H NMR (400 MHz, DMSO-d₆, δ): 8.63 (d, J=2.45 Hz, 1H), 8.45 (br s, 2H), 8.09 (dd, J=9.29, 2.45 Hz, 1H), 6.88 (d, J=9.29 Hz, 1H), 2.62 (s, 3H).

### 2. Synthesis of N-(2-acetyl-4-nitrophenyl)-1-methyl-1H-pyrazole-4-carboxamide

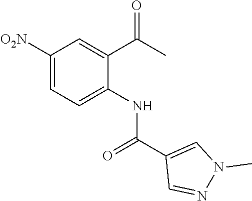 [SMILES: CC(=O)c1cc([N+](=O)[O-])ccc1NC(=O)c1cnn(C)c1]

A mixture of 1-methyl-1H-pyrazole-4-carboxylic acid (2.0 g, 15.87 mmol) in POCl₃ (20 mL) was heated to 40° C. To this mixture was added 1-(2-amino-5-nitrophenyl)ethan-1-one (2.5 g, 13.88 mmol) at the same temperature. The reaction mixture was heated to 80° C. and stirred for 6 h. Then, the reaction mixture was poured into ice-cold water (100 mL). The solid precipitated was filtered, washed with water (50 mL), acetonitrile (20 mL), dried in vacuo to afford N-(2-acetyl-4-nitrophenyl)-1-methyl-1H-pyrazole-4-carboxamide (2.5 g, yield: 62%) as brown solid. LCMS (ESI+): m/z calcd. for C₁₃H₁₃N₄O₄ [M+H]⁺, 289; found, 289. ¹H NMR (400 MHz, DMSO-d₆, δ): 12.17 (s, 1H), 8.79 (d, J=2.4 Hz, 1H), 8.72 (d, J=9.3 Hz, 1H), 8.50 (dd, J=2.4, 9.3 Hz, 1H), 8.40 (s, 1H), 7.96 (s, 1H), 3.94 (s, 3H), 2.79 (s, 3H).

### 3. Synthesis of 2-(1-methyl-1H-pyrazol-4-yl)-6-nitroquinolin-4-ol

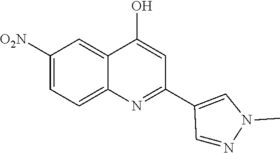 [SMILES: Cn1cc(-c2cc(O)c3cc([N+](=O)[O-])ccc3n2)cn1]

To a stirring solution of N-(2-acetyl-4-nitrophenyl)-1-methyl-1H-pyrazole-4-carboxamide (1.0 g, 3.46 mmol) in t-BuOH (10 mL) was added ^{t}BuOK (1.5 g, 6.9 mmol) at RT and the reaction mixture was heated to 80° C. and stirred for 6 h. Then, the reaction mixture filtered and the solid obtained was diluted with 1N aqueous HCl (10 mL). The solid precipitated was filtered, washed with water (10 mL), acetonitrile (20 mL), dried in vacuo to afford 2-(1-methyl-1H-pyrazol-4-yl)-6-nitroquinolin-4-ol (0.8 g, yield: 86%) as brown solid. LCMS (ESI+): m/z calcd. for C₁₃H₁₁N₄O₃[M+H]⁺, 271; found, 271. ¹H NMR (400 MHz, DMSO-d₆, δ): 12.04 (br s, 1H), 8.81 (br d, J=2.4 Hz, 1H), 8.54 (s, 1H), 8.44 (br dd, J=9.3 Hz, 2.4, 1H), 8.22 (s, 1H), 7.93 (br d, J=9.3 Hz, 1H), 6.55 (s, 1H), 3.94 (s, 3H).

### 4. Synthesis of 4-(2-methoxyethoxy)-2-(1-methyl-1H-pyrazol-4-yl)-6-nitroquinoline

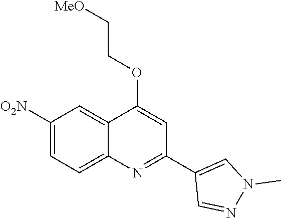 [SMILES: COCCOc1cc(-c2cnn(C)c2)nc2ccc([N+](=O)[O-])cc12]

4-(2-Methoxyethoxy)-2-(1-methyl-1H-pyrazol-4-yl)-6-nitroquinoline was prepared using the procedure followed for preparation of 4-ethoxy-6-nitroquinolin-2-ol. The titled compound was obtained as brown solid (0.8 g, crude was used for the next step without purification). LCMS (ESI+): m/z calcd. for C₁₆H₁₇N₄O₄ [M+H]⁺, 329; found, 329. ¹H NMR (400 MHz, DMSO-d₆, δ): 8.87 (br s, 1H), 8.58 (br s, 1H), 8.40 (br d, J=9.3 Hz, 1H), 8.26 (br s, 1H), 8.00 (br d, J=8.8 Hz, 1H), 7.95 (br s, 1H), 4.55 (br s, 2H), 3.99-3.91 (m, 2H), 3.89 (br s, 3H), 3.41 (s, 3H).

### 5. Synthesis of 4-(2-methoxyethoxy)-2-(1-methyl-1H-pyrazol-4-yl)quinolin-6-amine

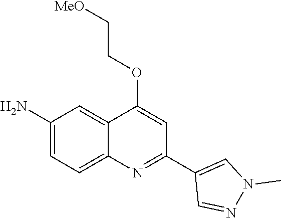 [SMILES: COCCOc1cc(-c2cnn(C)c2)nc2ccc(N)cc12]

4-(2-Methoxyethoxy)-2-(1-methyl-1H-pyrazol-4-yl)quinolin-6-amine was prepared using the procedure followed for preparation of 4-(2-methoxyethoxy)-2-(thiazol-5-yl) quinolin-6-amine as brown solid (0.6 g, crude was used for the next step without purification). LCMS (ESI+): m/z calcd. for C₁₆H₁₉N₄O₂ [M+H]⁺, 299; found, 299. ¹H NMR (400 MHz, DMSO-d₄, δ): 8.87 (br s, 1H), 8.58 (br s, 1H), 8.40 (br d, J=9.3 Hz, 1H), 8.26 (br s, 0.5H), 8.00 (br d, J=8.8 Hz, 1H), 7.95 (br s, 0.5H), 7.51 (br s, 1H), 4.55 (br s, 2H), 4.00-3.92 (m, 2H), 3.89 (br s, 2H), 3.41 (s, 6H).

### 6. Synthesis of N-(4-(2-methoxyethoxy)-2-(1-methyl-1H-pyrazol-4-yl)quinolin-6-yl)oxetane-3-carboxamide

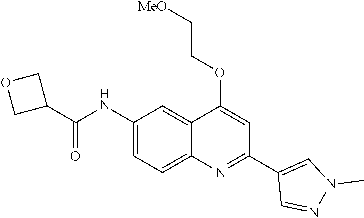 [SMILES: COCCOc1cc(-c2cnn(C)c2)nc2ccc(NC(=O)C3COC3)cc12]

N-(4-(2-methoxyethoxy)-2-(1-methyl-1H-pyrazol-4-yl)quinolin-6-yl)oxetane-3-carboxamide was prepared following the procedure of N-(4-ethoxy-2-(4-methyl-1H-imidazol-1-yl)quinolin-6-yl)oxetane-3-carboxamide as an off white solid (0.02 g, yield: 85%). HPLC purity: 93.43%. LCMS (ESI+): m/z calcd. for C₂₀H₂₃N₄O₄ [M+H]⁺, 383; found, 383. ¹H NMR (400 MHz, DMSO-d₆, δ): 10.21 (s, 1H), 8.43 (s, 1H), 8.38 (d, J=2.3 Hz, 1H), 8.13 (s, 1H), 7.92 (dd, J=9.1, 2.4 Hz, 1H), 7.80 (d, J=9.1 Hz, 1H), 7.28 (s, 1H), 4.75-4.71 (m, 4H), 4.46-4.44 (m, 2H), 4.05-3.96 (m, 1H), 3.91 (s, 3H), 3.87-3.82 (m, 2H), 3.40 (s, 3H).

### Example 54: N-(4-ethoxy-8-methyl-2-(4-methyl-1H-imidazol-1-yl)quinolin-6-yl)oxetane-3-carboxamide

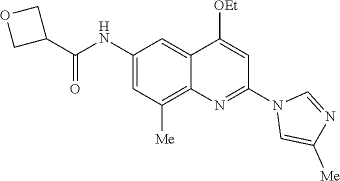 [SMILES: CCOc1cc(-n2cnc(C)c2)nc2c(C)cc(NC(=O)C3COC3)cc12]

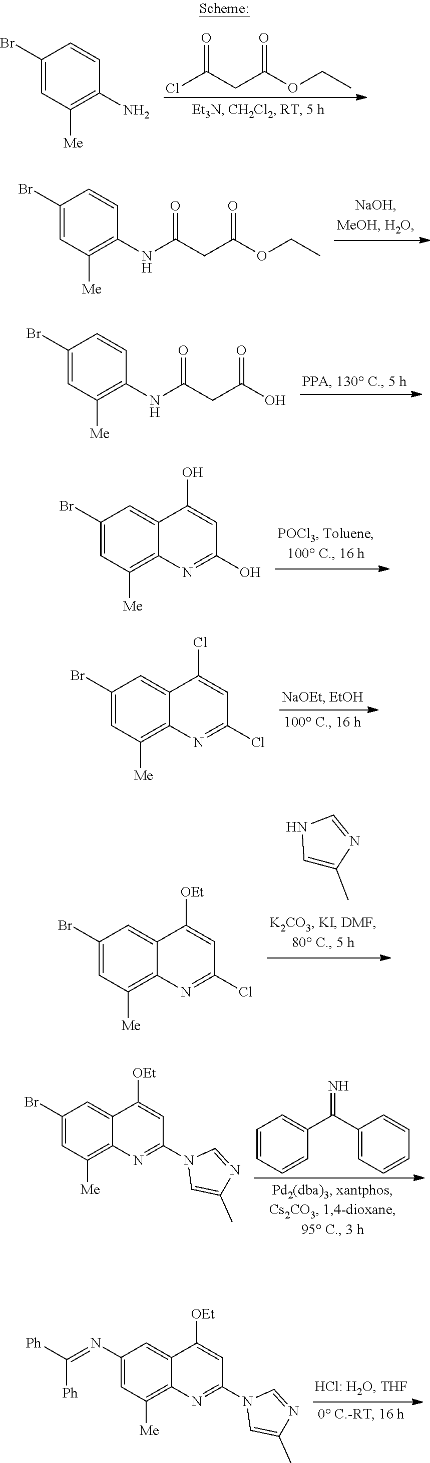

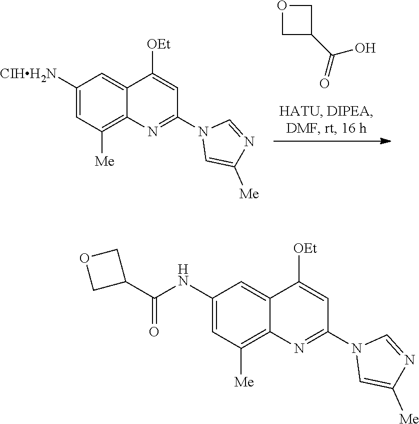

### 1. Synthesis of ethyl 3-((4-bromo-2-methylphenyl)amino)-3-oxopropanoate

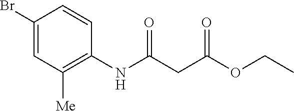 [SMILES: CCOC(=O)CC(=O)Nc1ccc(Br)cc1C]

To a stirred solution of 4-bromo-2-methylaniline (6 g, 32.25 mmol) in CH₂Cl₂ (60 mL) was added Et₃N (6.5 mL, 38.00 mmol) at RT. To this was added ethyl 3-chloro-3-oxopropanoate (3 mL, 96.00 mmol) at 0° C. The reaction mixture warmed to RT and stirred for 5 h. Then, the reaction mixture was diluted with ice cold water (200 mL) and extracted with CH₂Cl₂ (2×100 mL). The combined organic layers were dried over anhydrous Na₂SO₄, filtered and concentrated in vacuo to afford ethyl 3-((4-bromo-2-methylphenyl)amino)-3-oxopropanoate (4.3 g, crude) as an off-white solid. This reaction was carried out on 2×3 g scale. The crude was taken froward for next step without further purification.

### 2. Synthesis of 3-((4-bromo-2-methylphenyl)amino)-3-oxopropanoic acid

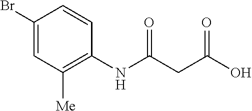 [SMILES: Cc1cc(Br)ccc1NC(=O)CC(=O)O]

To a stirred solution of ethyl 3-((4-bromo-2-methylphenyl)amino)-3-oxopropanoate (4.3 g, crude) in MeOH (20 mL) was added NaOH (2.29 g, 57.30 mmol) dissolved in H₂O (10 mL) at RT and stirred for 16 h. Then, the reaction mixture was diluted with ice cold water (100 mL) and washed with EtOAc (2×50 mL). The pH of the aqueous layer was acidified with aqueous citric acid to pH 5-6 and extracted with EtOAc (2×50). The organic extracts were dried over anhydrous Na₂SO₄, filtered and concentrated in vacuo to afford 3-((4-bromo-2-methylphenyl)amino)-3-oxopropanoic acid (1.5 g, yield: 17% over 2 steps) as an off-white solid. ¹H NMR (400 MHz, DMSO-d₆, δ): 12.64 (br d, J=7.34 Hz, 1H), 9.55 (s, 1H), 7.42-7.46 (m, 2H), 7.31-7.38 (m, 1H), 3.40 (s, 2H), 2.21 (s, 3H).

### 3. Synthesis of 6-bromo-8-methylquinoline-2,4-diol

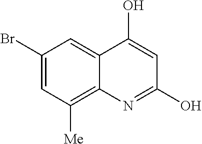 [SMILES: Cc1cc(Br)cc2c(O)cc(O)nc12]

A mixture of 3-((4-bromo-2-methylphenyl) amino)-3-oxopropanoic acid (3 g, 11.02 mmol) and PPA (7.4 g, 21.89 mmol) was heated to 130° C. and stirred for 5 h. Then, the reaction mixture was quenched with ice cold water, the solid obtained was filtered and dried under vacuum to afford 6-bromo-8-methylquinoline-2,4-diol (2.3 g, yield: 82%) as an off-white solid. This reaction was carried out on 2×1.5 g scale. LCMS (ESI+): m/z calcd. for C₁₀H₉BrNO₂ [M+H]⁺, 254; found, 254. ¹H NMR (400 MHz, DMSO-d₆, δ): 11.51 (br s, 1H), 10.50 (br s, 1H), 7.74 (s, 1H), 7.53 (s, 1H), 5.78 (s, 1H), 2.39 (s, 3H).

### 4. Synthesis of 6-bromo-2,4-dichloro-8-methylquinoline

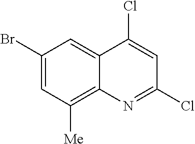 [SMILES: Cc1cc(Br)cc2c(Cl)cc(Cl)nc12]

6-Bromo-2,4-dichloro-8-methylquinoline was prepared using the procedure followed for preparation of 2,4-dichloro-6-nitroquinoline as a brown solid (1.3 g, yield: 49%). ¹H NMR (400 MHz, DMSO-d₆, δ) 8.19 (s, 1H), 8.04 (s, 1H), 8.00 (s, 1H), 2.67 (s, 3H).

### 5. Synthesis of 6-bromo-2-chloro-4-ethoxy-8-methylquinoline

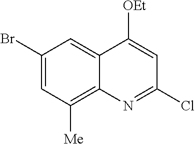 [SMILES: CCOc1cc(Cl)nc2c(C)cc(Br)cc12]

A stirred solution of 6-bromo-2,4-dichloro-8-methylquinoline (2.3 g, 7.90 mmol) in EtOH (10 mL) was added NaOEt (1.6 g, 23.52 mmol) at RT. The reaction mixture was stirred at RT and for 16 h. Then, the volatiles were removed in vacuo. The crude was diluted with NH₄Cl (50 mL). The solid obtained was filtered and dried under vacuum to afford 6-bromo-2-chloro-4-ethoxy-8-methylquinoline (1.3 g, yield: 61%) as an off-white solid. LCMS (ESI+): m/z calcd. for C₁₂H₁₂BrClNO [M+H]⁺, 300; found, 300. ¹H NMR (400 MHz, DMSO-d₆, δ): 8.08 (d, J=1.96 Hz, 1H), 7.83 (s, 1H), 7.15 (s, 1H), 4.35 (q, J=6.85 Hz, 2H), 2.61 (s, 3H), 1.47 (t, J=6.85 Hz, 3H).

### 6. Synthesis of 6-bromo-4-ethoxy-8-methyl-2-(4-methyl-1H-imidazol-1-yl)quinoline

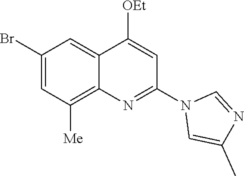 [SMILES: CCOc1cc(-n2cnc(C)c2)nc2c(C)cc(Br)cc12]

6-Bromo-4-ethoxy-8-methyl-2-(4-methyl-1H-imidazol-1-yl)quinoline was prepared using the procedure followed for preparation of 4-ethoxy-2-(4-methyl-1H-imidazol-1-yl)-6-nitroquinoline as an off-white solid (0.6 g, crude). LCMS (ESI+): m/z calcd. for C₁₆H₁₇BrN₃O [M+H]⁺, 346; found, 346.

### 6. Synthesis of N-(4-ethoxy-8-methyl-2-(4-methyl-1H-imidazol-1-yl)quinolin-6-yl)-1,1-diphenylmethanimine

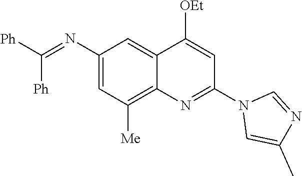 [SMILES: CCOc1cc(-n2cnc(C)c2)nc2c(C)cc(N=C(c3ccccc3)c3ccccc3)cc12]

To a stirred solution of 6-bromo-4-ethoxy-8-methyl-2-(4-methyl-1H-imidazol-1-yl)quinoline (0.50 g, 1.44 mmol) in 1,4-dioxane (3 mL) were added diphenylmethanimine, Cs₂CO₃ (1.17 g, 1.58 mmol) at RT and purged with N₂ for 20 min. To this were added Xantphos (0.167 g, 0.28 mmol) and Pd₂(dba)₃ (0.13 g, 0.14 mmol) were added and purged with N₂ for another 5 min. The reaction was heated at 110° C. for 16 h. Then, the reaction mixture was cooled to RT and diluted with ice-cold water (50 mL). The solid obtained was filtered and dried under vacuum. aqueous layer was extracted with EtOAc (2×100 mL). The combined organic layer was washed with brine (100 mL), dried over anhydrous Na₂SO₄, filtered and concentrated in vacuo. The crude was purified by silica gel (100-200 mesh) flash column chromatography (50% EtOAc in heptane) to afford N-(4-ethoxy-8-methyl-2-(4-methyl-1H-imidazol-1-yl)quinolin-6-yl)-1,1-diphenylmethanimine (0.4 g, yield: 62%) as yellow solid. LCMS (ESI+): m/z calcd. for C₂₉H₂₇N₄O [M+H]⁺, 447; found, 447.

### 7. Synthesis of 4-ethoxy-8-methyl-2-(4-methyl-1H-imidazol-1-yl)quinolin-6-amine hydrochloride

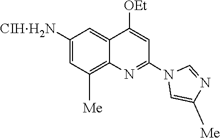

To a stirred solution of N-(4-ethoxy-8-methyl-2-(4-methyl-1H-imidazol-1-yl)quinolin-6-yl)-1,1-diphenylmethanimine (0.10 g, 0.22 mmol) in THF (5 mL) was added HCl:H₂O (1:1, 2 mL) at 0° C. The reaction mixture was warmed to RT and stirred for 2 h. Then, the volatiles were removed in vacuo to obtain the 4-ethoxy-8-methyl-2-(4-methyl-1H-imidazol-1-yl)quinolin-6-amine hydrochloride (0.09 g, crude) as pale brown solid.

### 8. Synthesis of N-(4-ethoxy-8-methyl-2-(4-methyl-1H-imidazol-1-yl)quinolin-6-yl)oxetane-3-carboxamide

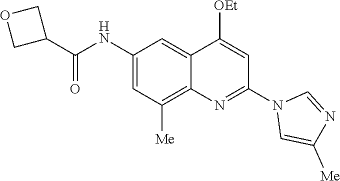 [SMILES: CCOc1cc(-n2cnc(C)c2)nc2c(C)cc(NC(=O)C3COC3)cc12]

N-(4-ethoxy-8-methyl-2-(4-methyl-1H-imidazol-1-yl)quinolin-6-yl)oxetane-3-carboxamide was prepared following the procedure of N-(4-ethoxy-2-(4-methyl-1H-imidazol-1-yl)quinolin-6-yl)oxetane-3-carboxamide as an off white solid (0.012 g, yield: 5%). HPLC purity: 95.87%. LCMS (ESI+): m/z calcd. for C₂₀H₂₃N₄O₃ [M+H]⁺, 367; found, 367. ¹H NMR (400 MHz, DMSO-d₆, δ): 10.16 (s, 1H), 8.61 (d, J=1.13 Hz, 1H), 8.33 (d, J=2.00 Hz, 1H), 7.85 (d, J=1.13 Hz, 1H), 7.80 (d, J=1.63 Hz, 1H), 7.32 (s, 1H), 4.74-4.72 (m, 4H), 4.43 (q, J=7.00 Hz, 2H), 4.02-4.39 (m, 1H), 2.65 (s, 3H), 2.21 (s, 3H), 1.51 (t, J=7.00 Hz, 3H).

### AlphaLISA Assay

Human DRIP1 (UniProtID: O00429.4, residues 1-699), and human MiD49 (UnitProtIS: Q96C03.1, residues 126-454) was cloned into a pET15b vector as an N-terminal His-tag Fusion. All constructs were transformed in Escherichia coli host strain BL21 CodonPlus DE3. Bacteria was grown in LB media with 100 μg/ml ampicillin and 34 μg/ml chloramphenicol in an orbital shaker at 37° C. until OD₆₀₀ reached 0.4, at which temperature was reduced to 16° C. Recombinant protein expression was expressed for 18 hr by the addition of IPTG at OD₆₀₀ of about 0.8. All cultures were spun down and bacterial pellets were used for protein purification.

For His-DRP1, the pellets were resuspended in DRP-buffer A (20 mM Tris, 500 KCl, 1.89 β-ME, pH 8.0) and protease inhibitor cocktail (Roche), followed by cell disruption by sonication (400 W, pulse 3 sec on, 3 sec off, total 20 min). The lysate was centrifuged at 20,000 g and then supernatant applied to a preequilibrated Ni-NTA column with DRP-buffer A. The column was extensively washed with buffer A containing 50 mM imidazole. Purified His-DRP1 was eluted with DRP-buffer A containing 250 mM imidazole and dialyzed overnight at 4° C. (18 kDa cutoff) against DRP-buffer A to remove imidazole. For AlphaLISA, the His tag was retained on DRP1 full-length.

For MiD49-GST, the bacterial pellets were resuspended and sonicated in MID lysis buffer (25 mM Tris-HCl, pH 7.5, 500 mM NaCl, 5% glycerol, 1 mM TCEP, and 0.5% CHAPS). The lysate was pre-cleared at 20,300 g before applying to a glutathione column. Unbound proteins were washed using MID-buffer A (25 mM Tris-HCl, pH 7.5, 300 mM NaCl, 5% glycerol, 1 mM TCEP) and MID-buffer B (25 mM Tris-HCl, pH 7.5, 300 mM NaCl, 5% glycerol, 1 mM TCEP, 1 mM GSH). The protein was eluted with MID-buffer C (25 mM Tris-HCl, pH 7.5, 300 mM NaCl, 5% glycerol, 1 mM TCEP, 10 mM GSH). The target protein was concentrated in the ultra-filtration tube with MW cutoff 30 kDa and further purified by size-exclusion chromatography on a Superdex-200 column (GE Healthcare) in buffer A. Fractions containing MiD49-GST (126-454) were pooled, concentrated, flash-frozen as single-use aliquots in liquid nitrogen and stored at −80° C.

A total of 100 nl of compounds were plated in a 384-well AlphaPlate (PerkinElmer, Waltham, MA) at concentrations 100 uM to 991 pM in DMSO (dilution by 3.165 for 11 concentration levels). Recombinant His-DRP1 (200 nM), MiD49-GST (20 nM), and GMP-PNP (100 uM) were mixed in assay buffer (PBS with 1.5% BSA, 1 mM DTT). Five microliters of the protein mixture were added to the plate and incubate at room temperature for 30 min. Glutathione donor beads and nickel chelate acceptor beads were diluted and mixed in assay buffer to obtain 40 μg/ml of each bead. Five microliters of the mixed beads were added to each well on the microplate leading to a final bead concentration of 20 g/ml per well. The sealed plate was incubated at room temperature for 60 min and protected from light. The AlphaLISA signal was detected with an EnVision Multilabel Reader (excitation at 680 and emission 615 nm). GraphPad Prism was used to calculate the IC₅₀ by applying sigmoidal dose-response fit (variable slope).

### DATA FOR EXAMPLES

| Example No. | DRP1 IC₅₀ (μM) |
|---|---|
| 1 | ++++ |
| 2 | ++++ |
| 3 | ++++ |
| 4 | +++ |
| 5 | +++ |
| 6 | ++++ |
| 7 | ++++ |
| 8 | +++ |
| 9 | ++++ |
| 10 | ++ |
| 11 | +++ |
| 12 | ++ |
| 13 | ++ |
| 14 | ++ |
| 15 | ++ |
| 16 | ++++ |
| 17 | ++++ |
| 18 | ++++ |
| 19 | +++ |
| 20 | ++ |
| 21 | ++ |
| 22 | ++++ |
| 23 | ++++ |
| 24 | +++ |
| 25 | ++++ |
| 26 | ++++ |
| 27 | +++ |
| 28 | ++++ |
| 29 | + |
| 30 | ++ |
| 31 | ++ |
| 32 | +++ |
| 33 | ++++ |
| 34 | + |
| 35 | +++ |
| 36 | ++++ |
| 37 | ++++ |
| 38 | ++++ |
| 39 | ++ |
| 40 | ++++ |
| 41 | ++++ |
| 42 | ++ |
| 43 | ++ |
| 44 | +++ |
| 45 | ++ |
| 46 | ++ |
| 47 | + |
| 48 | +++ |
| 49 | ++ |
| 50 | ++ |
| 51 | + |
| 52 | ++ |
| 53 | +++ |
| 54 | ++ |

“++++” means <0.5 μM;
“+++” means 0.5-1 μM;
“++” means >1-10 μM;
“+” means >10 μM;
“NT” means “Not Tested”.

1. A compound represented by Formula (I): 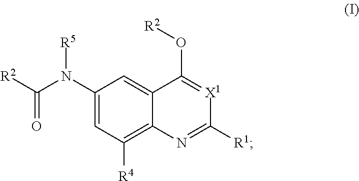
or a pharmaceutically acceptable salt thereof, wherein:
X¹ is CH or N;
R¹ is a 5- to 10-membered monocyclic or bicyclic heteroaryl optionally substituted with one more R^{1a};
each R^{1a} is independently selected from the group consisting of halo, cyano, C₁₋₆alkyl, C₁₋₆haloalkyl, C₃₋₆cycloalkyl, OH, C₁₋₆alkoxy, and C₁₋₆haloalkoxy;
R² is C₁₋₆alkyl, C₃₋₆cycloalkyl, bridged C₅₋₁₂cycloalkyl, phenyl, 3- to 10-membered monocyclic or bicyclic heterocyclyl, or 5- to 10-membered monocyclic or bicyclic heteroaryl, each of which is optionally substituted by one or more R^{2a};
each R^{2a} is independently selected from halo, cyano, C₁₋₆alkyl, C₁₋₆haloalkyl, OH, C₁₋₆alkoxy, C₁₋₆haloalkoxy, —C(O)R^{2b}, —C(O)OR^{2b}, —C(O)NR^{N2a}R^{N2b}, and —NR^{N2a}R^{N2b};
R^{2b}, R^{N2a}, and R^{N2b} are each independently H, C₁₋₆alkyl, or C₁₋₆haloalkyl;
R³ is C₁₋₆alkyl, C₃₋₆cycloalkyl, phenyl, 3- to 10-membered monocyclic or bicyclic heterocyclyl, or 5- to 10-membered monocyclic or bicyclic heteroaryl, each of which is optionally substituted with one or more R^{3a};
each R^{3a} is independently selected from the group consisting of halo, C₁₋₆alkyl, OH, C₁₋₆alkoxy, and C₁₋₆haloalkoxy;
R⁴ is H, C₁₋₆alkyl, C₁₋₆haloalkyl, or C₃₋₆cycloalkyl; and
R⁵ is H, C₁₋₆alkyl, or C₁₋₆haloalkyl.

2. The compound of claim 1, wherein the compound is represented by Formula (IA): 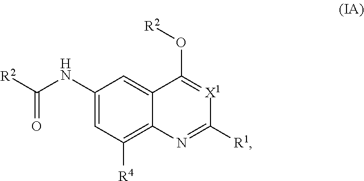
or a pharmaceutically acceptable salt thereof.

3. The compound of claim 2, or a pharmaceutically acceptable salt thereof, wherein R⁴ is H or C₁₋₃alkyl.

4. The compound of claim 3, or a pharmaceutically acceptable salt thereof, wherein R⁴ is H or —CH₃.

5. The compound of claim 2, or a pharmaceutically acceptable salt thereof, wherein:
R¹ is a 5- or 6-membered monocyclic heteroaryl, wherein the 5- or 6-membered monocyclic heteroaryl is optionally substituted by 1 or 2 R^{1a};
each R^{1a} is independently selected from the group consisting of halo, cyano, C₁₋₄alkyl, C₃₋₄cycloalkyl, and C₁₋₃alkoxy.

6. The compound of claim 5, or a pharmaceutically acceptable salt thereof, wherein R¹ is selected from the group consisting of imidazoyl, pyrazoyl, triazoyl, and thiazoyl, each of which is optionally substituted by 1 or 2 R^{1a}.

7. The compound of claim 6, or a pharmaceutically acceptable salt thereof, wherein R¹ is represented by the following structural formula: 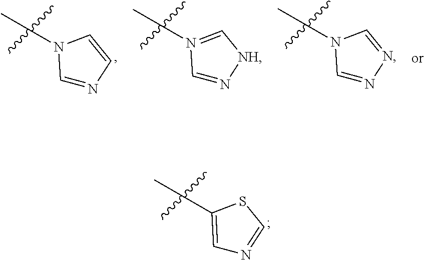
each of which is optionally substituted by 1 or 2 R^{1a}, and wherein each R^{1a} is independently selected from the group consisting of —CH₃, —CH₂CH₃, —CH₂CH₂CH₃, —C(CH₃)₃, cyclopropyl, —OCH₃, —Cl, and cyano.

8. The compound of claim 5, or a pharmaceutically acceptable salt thereof, wherein R¹ is selected from the group consisting of imidazoyl and thiazoyl, each of which is optionally substituted by 1 or 2 R^{1a}, and wherein each R^{1a} is independently selected from the group consisting of —CH₃, cyclopropyl, and —OCH₃.

9. The compound of claim 2, or a pharmaceutically acceptable salt thereof, wherein:
R² is C₁₋₃alkyl, C₃₋₆cycloalkyl, bridged C₅₋₈cycloalkyl, 3- to 6-membered monocyclic heterocyclyl, or 5- or 6-membered monocyclic heteroaryl, each of which is optionally substituted by 1 to 3 R^{2a};
each R^{2a} is independently selected from the group consisting of halo, cyano, C₁₋₃alkyl, C₁₋₃haloalkyl, —OH and C₁₋₃alkoxy.

10. The compound of claim 9, or a pharmaceutically acceptable salt thereof, wherein R² is C₁₋₃alkyl, cyclopropyl, cyclobutyl, bicyclo[1.1.1]pentanyl, oxetanyl, tetrahydropyranyl, or isoxazoyl, each of which is optionally substituted by 1 to 3 R^{2a}.

11. The compound of claim 10, or a pharmaceutically acceptable salt thereof, wherein R² is C₁₋₃alkyl optionally substituted by 1 to 3 R^{2a}, or R² is represented by the following structural formula: 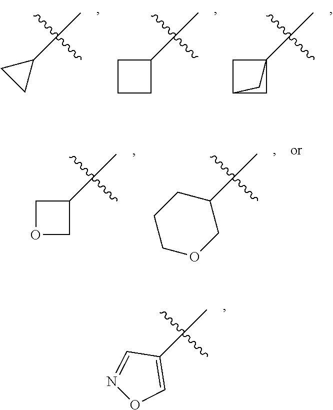
each of which is optionally substituted by 1 or 2 R^{2a}, and wherein each R^{2a} is selected from the group consisting of —F, cyano, —CH₃, —CF₃, —OH, —OCH₃, —OCH₂CH₃, and —OCH₂CH₂CH₃.

12. The compound of claim 11, or a pharmaceutically acceptable salt thereof, wherein R² is —CF₃, —CH₂CH₂OCH₃, —CH₂OCH₃, —CH₂OH, —CH(OH)CH₃, —CH(CH₃)CH₂OCH₃, —CH(CH₃)CN, —CH(CH₃)OCH₃, —CH(CH₃)OCH₂CH₃, —CH(CH₃)OCH₂CH₂CH₃, —CF₂CH₂OH, or R² is represented by the following structural formula:

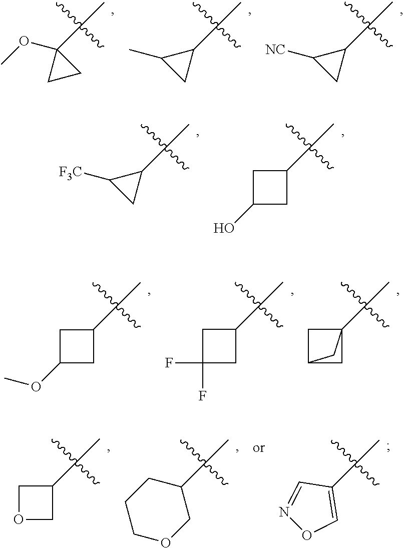

13. The compound of claim 9, or a pharmaceutically acceptable salt thereof, wherein R² is C₁₋₃alkyl, cyclopropyl, cyclobutyl, oxetanyl, or tetrahydropyranyl, wherein the C₁₋₃alkyl, cyclopropyl, and cyclobutyl are each optionally substituted with 1 to 3 R^{2a}.

14. The compound of claim 13, or a pharmaceutically acceptable salt thereof, wherein R² is —CH(CH₃)CN, —CH(CH₃)OCH₃, —CH(CH₃)OCH₂CH₃, —CH(CH₃)OCH₂CH₂CH₃, or R² is represented by the following structural formula:

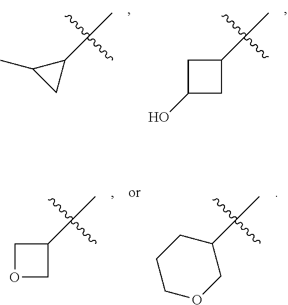

15. The compound of claim 2, or a pharmaceutically acceptable salt thereof, wherein:
R³ is C₁₋₄alkyl or C₃₋₄cycloalkyl, each of which is optionally substituted with 1 to 3 R^{3a};
each R^{3a} is independently selected from the group consisting of C₁₋₃alkyl, —OH, and C₁₋₃alkoxy.

16. The compound of claim 15, or a pharmaceutically acceptable salt thereof, wherein:
R³ is C₁₋₄alkyl or cyclopropyl, and wherein the C₁₋₃alkyl is optionally substituted with 1 to 3 R^{3a};
each R^{3a} is independently selected from —CH₃, OH, and —OCH₃.

17. The compound of claim 16, or a pharmaceutically acceptable salt thereof, wherein R³ is —CH₃, —CH₂CH₃, —CH₂CH₂OH, —CH₂CH₂OCH₃, —CH(CH₃)CH₂OCH₃, —C(CH₃)₂CH₂OCH₃, or cyclopropyl.

18. The compound of claim 1, or a pharmaceutically acceptable salt thereof, wherein the compound is selected from: [chemical structure] [chemical structure]
or a pharmaceutically acceptable salt thereof.

19. A pharmaceutical composition comprising a compound of claim 1, or a pharmaceutically acceptable salt thereof, and a pharmaceutically acceptable excipient.

20. A method of treating a Dynamin-1-like protein (Drp1) mediated disease or disorder in a subject, comprising administering to the subject an effective amount of a compound, or pharmaceutically acceptable salt thereof, of claim 1.

21-26. (canceled)
